SEQUENCE LISTING (75 sequences)

SEQ ID NO: 1 - DNA, 1308 bp, Homo sapiens
  CDS (157)..(1122)
  misc_feature (1): N = A or C or G or T
ntggttgttggaccattaaaatgcattatggaatttttaaaagttgggggagagggagacagtaaaaataacctatattttctcttgtttttttttttttaactctaggaaagcccagacaaattttgagctatttcataacctaccagacttatcatgctaacactgaataaaacagacctaataccagcttcatttattctgaatggagtcccaggactggaagacacacaactctggatttccttcccattctgctctatgtatgttgtggctatggtagggaattgtggactcctctacctcattcactatgaggatgccctgcacaaacccatgtactacttcttggccatgctttcctttactgaccttgttatgtgctctagtacaatccctaaagccctctgcatcttctggtttcatctcaaggacattggatttgatgaatgccttgtccagatgttcttcatccacaccttcacagggatggagtctggggtgcttatgcttatggccctggatcgctatgtggccatctgctaccccttacgctattcaactatcctcaccaatcctgtaattgcaaaggttgggactgccaccttcctgagaggggtattactcattattccctttactttcctcaccaagcgcctgccctcctgcagaggcaatatacttccccatacctactgtgaccacatgtctgtagccaaattgtcctgtggtaatgtcaaggtcaatgccatctatggtctgatggttgccctcctgattgggggctttgacatactgtgtatcaccatctcctataccatgattctccgggcagtggtcagcctctcctcagcagatgctcggcagaaggcctttaatacctgcactgcccacatttgtgccattgttttctcctatactccagctttcttctccttcttttcccaccgctttggggaacacataatccccccttcttgccacatcattgtagccaatatttatctgctcctaccacccactatgaaccctattgtctatggggtgaaaaccaaacagatacgagactgtgtcataaggatcctttcaggttctaaggataccaaatcctacagcatgtgaatgaacacttgccaggagtgagaagagaaggaaagaattacttctatttgcctcttatgcaggagttcataaaatctttctggaagtactgtattgatcacaaaatggagtttgntgactggtgcattctcaataagtaccttgggaatctnacatcactggaaggcccaccacatttctataaat

SEQ ID NO: 2 - protein, 321 aa, Homo sapiens
MLTLNKTDLIPASFILNGVPGLEDTQLWISFPFCSMYVVAMVGNCGLLYLIHYEDALHKPMYYFLAMLSFTDLVMCSSTIPKALCIFWFHLKDIGFDECLVQMFFIHTFTGMESGVLMLMALDRYVAICYPLRYSTILTNPVIAKVGTATFLRGVLLIIPFTFLTKRLPSCRGNILPHTYCDHMSVAKLSCGNVKVNAIYGLMVALLIGGFDILCITISYTMILRAVVSLSSADARQKAFNTCTAHICAIVFSYTPAFFSFFSHRFGEHIIPPSCHIIVANIYLLLPPTMNPIVYGVKTKQIRDCVIRILSGSKDTKSYSM

SEQ ID NO: 3 - DNA, 1014 bp, Homo sapiens
  CDS (1)..(1014)
atggatgaaacaggaaatctgacagtatcttctgccacatgccatgacactattgatgacttccgcaatcaagtgtattccaccttgtactctatgatctctgttgtaggcttctttggcaatggctttgtgctctatgtcctcataaaaacctatcacaagaagtcagccttccaagtatacatgattaatttagcagtagcagatctactttgtgtgtgcacactgcctctccgtgtggtctattatgttcacaaaggcatttggctctttggtgacttcttgtgccgcctcagcacctatgctttgtatgtcaacctctattgtagcatcttctttatgacagccatgagctttttccggtgcattgcaattgtttttccagtccagaacattaatttggttacacagaaaaaagccaggtttgtgtgtgtaggtatttggatttttgtgattttgaccagttctccatttctaatggccaaaccacaaaaagatgagaaaaataataccaagtgctttgagcccccacaagacaatcaaactaaaaatcatgttttggtcttgcattatgtgtcattgtttgttggctttatcatcccttttgttattataattgtctgttacacaatgatcattttgaccttactaaaaaaatcaatgaaaaaaaatctgtcaagtcataaaaaggctataggaatgatcatggtcgtgaccgctgcctttttagtcagtttcatgccatatcatattcaacgtaccattcaccttcattttttacacaatgaaactaaaccctgtgattctgtccttagaatgcagaagtccgtggtcataaccttgtctctggctgcatccaattgttgctttgaccctctcctatatttcttttctgggggtaactttaggaaaaggctgtctacatttagaaagcattctttgtccagcgtgacttatgtacccagaaagaaggcctctttgccagaaaaaggagaagaaatatgtaaagtatag

SEQ ID NO: 4 - protein, 337 aa, Homo sapiens
MDETGNLTVSSATCHDTIDDFRNQVYSTLYSMISVVGFFGNGFVLYVLIKTYHKKSAFQVYMINLAVADLLCVCTLPLRVVYYVHKGIWLFGDFLCRLSTYALYVNLYCSIFFMTAMSFFRCIAIVFPVQNINLVTQKKARFVCVGIWIFVILTSSPFLMAKPQKDEKNNTKCFEPPQDNQTKNHVLVLHYVSLFVGFIIPFVIIIVCYTMIILTLLKKSMKKNLSSHKKAIGMIMVVTAAFLVSFMPYHIQRTIHLHFLHNETKPCDSVLRMQKSVVITLSLAASNCCFDPLLYFFSGGNFRKRLSTFRKHSLSSVTYVPRKKASLPEKGEEICKV

SEQ ID NO: 5 - DNA, 2429 bp, Homo sapiens
  CDS (691)..(1845)
ggggcctacttcaccgtgtacccggacttgggaccatcacagacttcagaaccatcaggaacctgggagcaactgaaagctgaactacagtgggctttcagacacacagcaggctgcggagcacaaataggactggttccctccaggccaccagcagggcggtggaggtcttcactgactccctgcctacctctcaggacaatgtccttttggctccacagtccctgaagccagagctggtgggggcagggaggcagccaccagcctctatatgtagtggaggagggggtgtccagggagggctgcatgatcctgagagcccccacctcacccggctggactatcctcccacttcagggtttctctgggcttccatcttgcccctgctgagccctgcttcctcctctaccagcagcacaacccccaggctgggctcagagacctcatgtggtgggatcactcagtaccccgaggcggagggaaggagggagggctgcagggttccccttggcctgcaaacaggaacacagggtgtttctcagtggctgcgagaatgctgatgaaaaccccaggatgttgtgtcaccgtggtggccagctgatagtgccaatcatcccactttgccctgagcactcctgcaggggtagaagactccagaaccttctctcaggcccatggcccaagcagcccatggaacttcataacctgagctctccatctccctctctctcctcctctgttctccctccctccttctctccctcaccctcctctgctccctctgcctttaccactgtgggggggtcctctggagggccctgccaccccacctcttcctcgctggtgtctgccttcctggcaccaatcctggccctggagtttgtcctgggcctggtggggaacagtttggccctcttcatcttctgcatccacacgcggccctggacctccaacacggtgttcctggtcagcctggtggccgctgacttcctcctgatcagcaacctgcccctccgcgtggactactacctcctccatgagacctggcgctttggggctgctgcctgcaaagtcaacctcttcatgctgtccaccaaccgcacggccagcgttgtcttcctcacagccatcgcactcaaccgctacctgaaggtggtgcagccccaccacgtgctgagccgtgcttccgtgggggcagctgcccgggtggccgggggactctgggtgggcatcctgctcctcaacgggcacctgctcctgagcaccttctccggcccctcctgcctcagctacagggtgggcacgaagccctcggcctcgctccgctggcaccaggcactgtacctgctggagttcttcctgccactggcgctcatcctctttgctattgtgagcattgggctcaccatccggaaccgtggtctgggcgggcaggcaggcccgcagagggccatgcgtgtgctggccatggtggtggccgtctacaccatctgcttcttgcccagcatcatctttggcatggcttccatggtggctttctggctgtccgcctgccgatccctggacctctgcacacagctcttccatggctccctggccttcacctacctcaacagtgtcctggaccccgtgctctactgcttctctagccccaacttcctccaccagagccgggccttgctgggcctcacgcggggccggcagggcccagtgagcgacgagagctcctaccaaccctccaggcagtggcgctaccgggaggcctctaggaaggcggaggccatagggaagctgaaagtgcagggcgaggtctctctggaaaaggaaggctcctcccagggctgagggccagctgcagggctgcagcgctgtgggggtaagggctgccgcgctctggcctggagggacaaggccagcacacggtgcctcaaccaactggacaagggatggcggcagaccaggggccaggccaaagcactggcaggactcatgtgggtggcagggagagaaacccacctaggcctctcagtgtgtccaggatggcattcccagaatgcaggggagagcaggatgccgggtggaggagacaggcaaggtgccgttggcacaccagctcagacaggggcctgcgcagctgcaggggacagacgccaatcactgtcacagcagagtcaccttagaaattggacagctgcatgttctgtgctctccagtttgtcccttccaatattaataaacttcccttttaaatatatttatttgcagaccaatatctgtctttaattctaacctgggactgtcagtaggcgtcaaagtgagcgccccagtgaaggaaccttggagagagtgggagcattcccagccttccagggggactcgtcttccagactttggagcccgcatgtctgaagcagactctttcttggtag

SEQ ID NO: 6 - protein, 384 aa, Homo sapiens
MELHNLSSPSPSLSSSVLPPSFSPSPSSAPSAFTTVGGSSGGPCHPTSSSLVSAFLAPILALEFVLGLVGNSLALFIFCIHTRPWTSNTVFLVSLVAADFLLISNLPLRVDYYLLHETWRFGAAACKVNLFMLSTNRTASVVFLTAIALNRYLKVVQPHHVLSRASVGAAARVAGGLWVGILLLNGHLLLSTFSGPSCLSYRVGTKPSASLRWHQALYLLEFFLPLALILFAIVSIGLTIRNRGLGGQAGPQRAMRVLAMVVAVYTICFLPSIIFGMASMVAFWLSACRSLDLCTQLFHGSLAFTYLNSVLDPVLYCFSSPNFLHQSRALLGLTRGRQGPVSDESSYQPSRQWRYREASRKAEAIGKLKVQGEVSLEKEGSSQG

SEQ ID NO: 7 - DNA, 1484 bp, Homo sapiens
  CDS (146)..(1147)
ttgaatttaggtgacactatagaagagctatgacgtcgcatgcacgcgtacgtaagctcggaattcggctcgagctgaactaatgactgccgccataagaagacagagagaactgagtatcctcccaaaggtgacactggaagcaatgaacaccacagtgatgcaaggcttcaacagatctgagcggtgccccagagacactcggatagtacagctggtattcccagccctctacacagtggttttcttgaccggcatcctgctgaatactttggctctgtgggtgtttgttcacatccccagctcctccaccttcatcatctacctcaaaaacactttggtggccgacttgataatgacactcatgcttcctttcaaaatcctctctgactcacacctggcaccctggcagctcagagcttttgtgtgtcgtttttcttcggtgatattttatgagaccatgtatgtgggcatcgtgctgttagggctcatagcctttgacagattcctcaagatcatcagacctttgagaaatatttttctaaaaaaacctgtttttgcaaaaacggtctcaatcttcatctgggtctttttggtcttcatctccctgccaaatatgatcttgagcaacaaggaagcaacaccatcgtctgtgaaaaagtgtgcttccttaaaggggcctctggggctgaaatggcatcaaatggtaaataacatatgccagtttattttctggactggttttatcctaatgcttgtgttttatgtggttattgcaaaaaaagtatatgattcttatagaaagtccaaaagtaaggacagaaaaaacaacaaaaagctggaaggcaaagtatttgttgtcgtggctgtcttctttgtgtgttttgctccatttcattttgccagagttccatatactcacagtcaaaccaacaataagactgactgtagactgcaaaatcaactgtttattgctaaagaaacaactctctttttggcagcaactaacatttgtatggatcccttaatatacatattcttatgtaaaaaattcacagaaaagctaccatgtatgcaagggagaaagaccacagcatcaagccaagaaaatcatagcagtcagacagacaacataaccttaggctgacaactgtacatagggttaacttctatttattgatgagacttccgtagataatgtggaaatcaaatttaaccaagaaaaaaagattggaacaaatgctctcttacattttatttatcctggtgtccaggaaaagattatattaaatttaaatccacatagatctattcataagctgaatgaaccattacctaagagaatgcaacaggataccaatggccactagaggcatattccttcttcttttttttttgttaaatttcaagagcattcactttacatttggaaagactaaggggaacggttatcctacaaacctcccttcaacaccttttacatt

SEQ ID NO: 8 - protein, 333 aa, Homo sapiens
MNTTVMQGFNRSERCPRDTRIVQLVFPALYTVVFLTGILLNTLALWVFVHIPSSSTFIIYLKNTLVADLIMTLMLPFKILSDSHLAPWQLRAFVCRFSSVIFYETMYVGIVLLGLIAFDRFLKIIRPLRNIFLKKPVFAKTVSIFIWVFLVFISLPNMILSNKEATPSSVKKCASLKGPLGLKWHQMVNNICQFIFWTGFILMLVFYVVIAKKVYDSYRKSKSKDRKNNKKLEGKVFVVVAVFFVCFAPFHFARVPYTHSQTNNKTDCRLQNQLFIAKETTLFLAATNICMDPLIYIFLCKKFTEKLPCMQGRKTTASSQENHSSQTDNITLG

SEQ ID NO: 9 - DNA, 957 bp, Homo sapiens
  CDS (1)..(954)
atgatggtggatcccaatggcaatgaatccagtgctacatacttcatcctaataggcctccctggtttagaagaggctcagttctggttggccttcccattgtgctccctctaccttattgctgtgctaggtaacttgacaatcatctacattgtgcggactgagcacagcctgcatgagcccatgtatatatttctttgcatgctttcaggcattgacatcctcatctccacctcatccatgcccaaaatgctggccatcttctggttcaattccactaccatccagtttgatgcttgtctgctacagatgtttgccatccactccttatctggcatggaatccacagtgctgctggccatggcttttgaccgctatgtggccatctgtcacccactgcgccatgccacagtacttacgttgcctcgtgtcaccaaaattggtgtggctgctgtggtgcggggggctgcactgatggcaccccttcctgtcttcatcaagcagctgcccttctgccgctccaatatcctttcccattcctactgcctacaccaagatgtcatgaagctggcctgtgatgatatccgggtcaatgtcgtctatggccttatcgtcatcatctccgccattggcctggactcacttctcatctccttctcatatctgcttattcttaagactgtgttgggcttgacacgtgaagcccaggccaaggcatttggcacttgcgtctctcatgtgtgtgctgtgttcatattctatgtacctttcattggattgtccatggtgcatcgctttagcaagcggcgtgactctccgctgcccgtcatcttggccaatatctatctgctggttcctcctgtgctcaacccaattgtctatggagtgaagacaaaggagattcgacagcgcatccttcgacttttccatgtggccacacacgcttcagagccctag

SEQ ID NO: 10 - protein, 318 aa, Homo sapiens
MMVDPNGNESSATYFILIGLPGLEEAQFWLAFPLCSLYLIAVLGNLTIIYIVRTEHSLHEPMYIFLCMLSGIDILISTSSMPKMLAIFWFNSTTIQFDACLLQMFAIHSLSGMESTVLLAMAFDRYVAICHPLRHATVLTLPRVTKIGVAAVVRGAALMAPLPVFIKQLPFCRSNILSHSYCLHQDVMKLACDDIRVNVVYGLIVIISAIGLDSLLISFSYLLILKTVLGLTREAQAKAFGTCVSHVCAVFIFYVPFIGLSMVHRFSKRRDSPLPVILANIYLLVPPVLNPIVYGVKTKEIRQRILRLFHVATHASEP

SEQ ID NO: 11 - DNA, 995 bp, Homo sapiens
  CDS (1)..(921)
atggaaagcgagaacagaagagtgataagagaattcatcctccttggtctgacccagtctcaagatattcagctcctggtctttgtgctagttttaatattctacttcatcatcctccctggaaattttctcattattttcaccataaagtcagaccctgggctcacagcccccctctatttctttctgggcaacttggccttcctggatgcatcctactccttcattgtggctccccggatgttggtggacttcctctctgcgaagaagataatctcctacagaggctgcatcactcagctctttttcttgcacttccttggaggaggggagggattactccttgttgtgatggcctttgaccgctacatcgccatctgccggcctctgcactatcctactgtcatgaaccctagaacctgctatgcaatgatgttggctctgtggcttgggggttttgtccactccattatccaggtggtcctcatcctccgcttgcctttttgtggcccaaaccagctggacaacttcttctgtgatgtcccacaggtcatcaagctggcctgcaccgacacatttgtggtggagcttctgatggtcttcaacagtggcctgatgacactcctgtgctttctggggcttctggcctcctatgcagtcattctttgtcgcatacgagggtcttcttctgaggcaaaaaacaaggccatgtccacgtgcatcacccatatcattgttatattcttcatgtttggacctggcatcttcatctacacgcgccccttcagggctttcccagctgacaaggtggtttctctcttccacacagtgatttttcctttgttgaatcctgtcatttatacccttcgcaaccaggaagtgaaagcttccatgaaaaaggtgtttaataagcacatagcctgaaaaagggcgcaaaaaaaaaaagaataaaaatagactgtagaatttttaaaaaaaaaaaaaaaaaaaaaaaa

SEQ ID NO: 12 - protein, 307 aa, Homo sapiens
MESENRRVIREFILLGLTQSQDIQLLVFVLVLIFYFIILPGNFLIIFTIKSDPGLTAPLYFFLGNLAFLDASYSFIVAPRMLVDFLSAKKIISYRGCITQLFFLHFLGGGEGLLLVVMAFDRYIAICRPLHYPTVMNPRTCYAMMLALWLGGFVHSIIQVVLILRLPFCGPNQLDNFFCDVPQVIKLACTDTFVVELLMVFNSGLMTLLCFLGLLASYAVILCRIRGSSSEAKNKAMSTCITHIIVIFFMFGPGIFIYTRPFRAFPADKVVSLFHTVIFPLLNPVIYTLRNQEVKASMKKVFNKHIA

SEQ ID NO: 13 - DNA, 1380 bp, Homo sapiens
  CDS (266)..(1375)
  misc_feature (32): n = A or C or G or T
tgcttccccataaggtaacagctttgttagcnctgtctgacatcattgcttgttnacttaagaactgataggtnttttttttttttttttttcagatattctgatggcaaaacaagtggaagaaaagaggaagcatgactgcagatcagatcagttctctttgtggattatattttcagtaaaatgtatggatctatcttttccttgttcttatatctagatcatgagacttgactgaggctgtatccttatcctccatccatctatggcgaactatagccatgcagctgacaacattttgcaaaatctctcgcctctaacagcctttctgaaactgacttccttgggtttcataataggagtcagcgtggtgggcaacctcctgatctccattttgctagtgaaagataagaccttgcatagagcaccttactacttcctgttggatctttgctgttcagatatcctcagatctgcaatttgtttcccatttgtgttcaactctgtcaaaaatggttctacctggacttatgggactctgacttgcaaagtgattgcctttctgggggttttgtcctgtttccacactgctttcatgctcttctgcatcagtgtcaccagatatttagctatcgcccatcaccgcttctatacaaagaggctgaccttttggacgtgtctggctgtgatctgtatggtgtggactctgtctgtggccatggcatttcccccggttttagacgtgggcacttactcattcattagggaggaagatcaatgcaccttccaacaccgctccttcagggctaatgattccttaggatttatgctgcttcttgctctcatcctcctagccacacagcttgtctacctcaagctgatatttttcgtccacgatcgaagaaaaatgaagccagtccagtttgtagcagcagtcagccagaactggacttttcatggtcctggagccagtggccaggcagctgccaattggctagcaggatttggaaggggtcccacaccacccaccttgctgggcatcaggcaaaatgcaaacaccacaggcagaagaaggctattggtcttagacgagttcaaaatggagaaaagaatcagcagaatgttctatataatgacttttctgtttctaaccttgtggggcccctacctggtggcctgttattggagagtttttgcaagagggcctgtagtaccagggggatttctaacagctgctgtctggatgagttttgcccaagcaggaatcaatccttttgtctgcattttctcaaacagggagctgaggcgctgtttcagcacaacccttctttactgcagaaaatccaggttaccaagggaaccttactgtgttatatgagg

SEQ ID NO: 14 - protein, 370 aa, Homo sapiens
MANYSHAADNILQNLSPLTAFLKLTSLGFIIGVSVVGNLLISILLVKDKTLHRAPYYFLLDLCCSDILRSAICFPFVFNSVKNGSTWTYGTLTCKVIAFLGVLSCFHTAFMLFCISVTRYLAIAHHRFYTKRLTFWTCLAVICMVWTLSVAMAFPPVLDVGTYSFIREEDQCTFQHRSFRANDSLGFMLLLALILLATQLVYLKLIFFVHDRRKMKPVQFVAAVSQNWTFHGPGASGQAAANWLAGFGRGPTPPTLLGIRQNANTTGRRRLLVLDEFKMEKRISRMFYIMTFLFLTLWGPYLVACYWRVFARGPVVPGGFLTAAVWMSFAQAGINPFVCIFSNRELRRCFSTTLLYCRKSRLPREPYCVI

SEQ ID NO: 15 - DNA, 1191 bp, Homo sapiens
  CDS (1)..(1188)
atgtttagacctcttgtgaatctctctcacatatattttaagaaattccagtactgtgggtatgcaccacatgttcgcagctgtaaaccaaacactgatggaatttcatctctagagaatctcttggcaagcattattcagagagtatttgtctgggttgtatctgcagttacctgctttggaaacatttttgtcatttgcatgcgaccttatatcaggtctgagaacaagctgtatgccatgtcaatcatttctctctgctgtgccgactgcttaatgggaatatatttattcgtgatcggaggctttgacctaaagtttcgtggagaatacaataagcatgcgcagctgtggatggagagtactcattgtcagcttgtaggatctttggccattctgtccacagaagtatcagttttactgttaacatttctgacattggaaaaatacatctgcattgtctatccttttagatgtgtgagacctggaaaatgcagaacaattacagttctgattctcatttggattactggttttatagtggctttcattccattgagcaataaggaatttttcaaaaactactatggcaccaatggagtatgcttccctcttcattcagaagatacagaaagtattggagcccagatttattcagtggcaatttttcttggtattaatttggccgcatttatcatcatagttttttcctatggaagcatgttttatagtgttcatcaaagtgccataacagcaactgaaatacggaatcaagttaaaaaagagatgatccttgccaaacgttttttctttatagtatttactgatgcattatgctggatacccatttttgtagtgaaatttctttcactgcttcaggtagaaataccaggtaccataacctcttgggtagtgatttttattctgcccattaacagtgctttgaacccaattctctatactctgaccacaagaccatttaaagaaatgattcatcggttttggtataactacagacaaagaaaatctatggacagcaaaggtcagaaaacatatgctccatcattcatctgggtggaaatgtggccactgcaggagatgccacctgagttaatgaagccggaccttttcacatacccctgtgaaatgtcactgatttctcaatcaacgagactcaattcctattcatga

SEQ ID NO: 16 - protein, 396 aa, Homo sapiens
MFRPLVNLSHIYFKKFQYCGYAPHVRSCKPNTDGISSLENLLASIIQRVFVWVVSAVTCFGNIFVICMRPYIRSENKLYAMSIISLCCADCLMGIYLFVIGGFDLKFRGEYNKHAQLWMESTHCQLVGSLAILSTEVSVLLLTFLTLEKYICIVYPFRCVRPGKCRTITVLILIWITGFIVAFIPLSNKEFFKNYYGTNGVCFPLHSEDTESIGAQIYSVAIFLGINLAAFIIIVFSYGSMFYSVHQSAITATEIRNQVKKEMILAKRFFFIVFTDALCWIPIFVVKFLSLLQVEIPGTITSWVVIFILPINSALNPILYTLTTRPFKEMIHRFWYNYRQRKSMDSKGQKTYAPSFIWVEMWPLQEMPPELMKPDLFTYPCEMSLISQSTRLNSYS

SEQ ID NO: 17 - DNA, 1164 bp, Homo sapiens
  CDS (13)..(1089)
cacaactgaagaatggggttcaacttgacgcttgcaaaattaccaaataacgagctgcacggccaagagagtcacaattcaggcaacaggagcgacgggccaggaaagaacaccacccttcacaatgaatttgacacaattgtcttgccagtgctttatctcattatatttgtggcaagcatcttgctgaatggtttagcagtgtggatcttcttccacattaggaataaaaccagcttcatattctatctcaaaaacatagtggttgcagacctcataatgacgctgacatttccatttcgaatagtccatgatgcaggatttggaccttggtacttcaagtttattctctgcagatacacttcagttttgttttatgcaaacatgtatacttccatcgtgttccttgggctgataagcattgatcgctatctgaaggtggtcaagccatttggggactctcggatgtacagcataaccttcacgaaggttttatctgtttgtgtttgggtgatcatggctgttttgtctttgccaaacatcatcctgacaaatggtcagccaacagaggacaatatccatgactgctcaaaacttaaaagtcctttgggggtcaaatggcatacggcagtcacctatgtgaacagctgcttgtttgtggccgtgctggtgattctgatcggatgttacatagccatatccaggtacatccacaaatccagcaggcaattcataagtcagtcaagccgaaagcgaaaacataaccagagcatcagggttgttgtggctgtgttttttacctgctttctaccatatcacttgtgcagaattccttttacttttagtcacttagacaggcttttagatgaatctgcacaaaaaatcctatattactgcaaagaaattacacttttcttgtctgcgtgtaatgtttgcctggatccaataatttactttttcatgtgtaggtcattttcaagaaggctgttcaaaaaatcaaatatcagaaccaggagtgaaagcatcagatcactgcaaagtgtgagaagatcggaagttctcatatattatgattatactgatgtgtaggccttttattgtttgttggaatcgatatgtacaaagtgtaaataaatgtttcttttcattaaaaaaaaaaaaaaa

SEQ ID NO: 18 - protein, 358 aa, Homo sapiens
MGFNLTLAKLPNNELHGQESHNSGNRSDGPGKNTTLHNEFDTIVLPVLYLIIFVASILLNGLAVWIFFHIRNKTSFIFYLKNIVVADLIMTLTFPFRIVHDAGFGPWYFKFILCRYTSVLFYANMYTSIVFLGLISIDRYLKVVKPFGDSRMYSITFTKVLSVCVWVIMAVLSLPNIILTNGQPTEDNIHDCSKLKSPLGVKWHTAVTYVNSCLFVAVLVILIGCYIAISRYIHKSSRQFISQSSRKRKHNQSIRVVVAVFFTCFLPYHLCRIPFTFSHLDRLLDESAQKILYYCKEITLFLSACNVCLDPIIYFFMCRSFSRRLFKKSNIRTRSESIRSLQSVRRSEVLIYYDYTDV

SEQ ID NO: 19 - DNA, 2480 bp, Homo sapiens
  CDS (42)..(1157)
catggcatccccagcctagctcccaatcccactttggcacgatgttagccaacagctcctcaaccaacagttctgttctcccgtgtcctgactaccgacctacccaccgcctgcacttggtggtctacagcttggtgctggctgccgggctccccctcaacgcgctagccctctgggtcttcctgcgcgcgctgcgcgtgcactcggtggtgagcgtgtacatgtgtaacctggcggccagcgacctgctcttcaccctctcgctgcccgttcgtctctcctactacgcactgcaccactggcccttccccgacctcctgtgccagacgacgggcgccatcttccagatgaacatgtacggcagctgcatcttcctgatgctcatcaacgtggaccgctacgccgccatcgtgcacccgctgcgactgcgccacctgcggcggccccgcgtggcgcggctgctctgcctgggcgtgtgggcgctcatcctggtgtttgccgtgcccgccgcccgcgtgcacaggccctcgcgttgccgctaccgggacctcgaggtgcgcctatgcttcgagagcttcagcgacgagctgtggaaaggcaggctgctgcccctcgtgctgctggccgaggcgctgggcttcctgctgcccctggcggcggtggtctactcgtcgggccgagtcttctggacgctggcgcgccccgacgccacgcagagccagcggcggcggaagaccgtgcgcctcctgctggctaacctcgtcatcttcctgctgtgcttcgtgccctacaacagcacgctggcggtctacgggctgctgcggagcaagctggtggcggccagcgtgcctgcccgcgatcgcgtgcgcggggtgctgatggtgatggtgctgctggccggcgccaactgcgtgctggacccgctggtgtactactttagcgccgagggcttccgcaacaccctgcgcggcctgggcactccgcaccgggccaggacctcggccaccaacgggacgcgggcggcgctcgcgcaatccgaaaggtccgccgtcaccaccgacgccaccaggccggatgccgccagtcaggggctgctccgaccctccgactcccactctctgtcttccttcacacagtgtccccaggattccgccctctgaacacacatgccattgcgctgtccgtgcccgactcccaacgcctctcgttctgggaggcttacagggtgtacacacaagaaggtgggctgggcacttggacctttgggtggcaattccagcttagcaacgcagaagagtacaaagtgtggaagccagggcccagggaaggcagtgctgctggaaatggcttctttaaactgtgagcacgcagagcaccccttctccagcggtgggaagtgatgcagagagcccacccgtgcagagggcagaagaggacgaaatgcctttgggtgggcagggcattaaactgctaaaagctggttagatggaacagaaaatgggcattctggatctaaaccgccacaggggcctgagagctgaagagcaccaggtttggtggacaaagctactgagatgcctgttcatctgctgacttctgtctaggctcatggatgccaccccctttcatttcggcctaggcttcccctgctcaccactgaggcctaatacaagagttcctatggacagaactacattctttctcgcatagtgacttgtgacaatttagacttggcatccagcatgggatagttggggcaaggcaaaactaacttagagtttccccctcaacaacatccaagtccaaaccctttttaggttatcctttcttccatcacatccccttttccaggcctcctccattttaggtccttaatattctttctttttctctctctctcgtttctctcttctctctcctctcctctcctctctcttctcctcttctctctctctccctctctctcctttgtccagagtaaggataaaattctttctactaaagcactggttctcaaactttttggtctcagaccccactcttagaaattgaggatctcaaagagctttgcttatattttgttcttttgatacttaccatactagaaattaaagcgaatacatttttaaaataaatacacatgcacacattacattagccatgggagcaataatgtcaccacacacacttcatgaagcctctggaaaactctacagtatacttgtgagagaatgagagtgaaagggacaaataacatctgtgtagcagtattatgaaaatagcttgaccttgtggacttcctcagagggttggtccctggatcacactttgagaaccatacttgtcctgaagtattggagttcatgtctaacttcttcccagggcattatgtacagtgctttttattactgtggggagagggcagtgctaaataaattaatcactactgataaaaaaaaaaaaaaaaaaaaaaaaaaaaa

SEQ ID NO: 20 - protein, 372 aa, Homo sapiens [sequence omitted]

SEQ ID NO: 21 - DNA, 19 bp, Artificial Sequence
  Description of Artificial Sequence Primer LW1282
taatacctgcactgcccac

SEQ ID NO: 22 - DNA, 22 bp, Artificial Sequence
  Description of Artificial Sequence Primer LW 1283
tctttccttctcttctcactcc

SEQ ID NO: 23 - DNA, 32 bp, Artificial Sequence
  Description of Artificial Sequence Primer LW 1373
gcataagcttatgctaacactgaataaaacag

SEQ ID NO: 24 - DNA, 30 bp, Artificial Sequence
  Description of Artificial Sequence Primer LW1374
gcatctcgagtcacatgctgtaggatttgg

SEQ ID NO: 25 - protein, 9 aa, Artificial Sequence
  Description of Artificial Sequence Peptide
APRTPGGRR

SEQ ID NO: 26 - DNA, 32 bp, Artificial Sequence
  Description of Artificial Sequence Primer LW1248
gcatgaattccaatatacttccccatacctac

SEQ ID NO: 27 - DNA, 30 bp, Artificial Sequence
  Description of Artificial Sequence Primer LW1249
gcatggatccggaaaagaaggagaagaaag

SEQ ID NO: 28 - DNA, 18 bp, Artificial Sequence
  Description of Artificial Sequence Primer LW1278
accgctgcctttttagtc

SEQ ID NO: 29 - DNA, 23 bp, Artificial Sequence
  Description of Artificial Sequence Primer LW1279
ccttctttctgggtacataagtc

SEQ ID NO: 30 - DNA, 30 bp, Artificial Sequence
  Description of Artificial Sequence Primer LW1405
aagcataacatggatgaaacaggaaatctg

SEQ ID NO: 31 - DNA, 29 bp, Artificial Sequence
  Description of Artificial Sequence Primer LW1406
aagcataactatactttacatatttcttc

SEQ ID NO: 32 - DNA, 22 bp, Artificial Sequence
  Description of Artificial Sequence Primer LW1280
tctgcacacagctcttccatgg

SEQ ID NO: 33 - DNA, 22 bp, Artificial Sequence
  Description of Artificial Sequence Primer LW1281
tcccttgtccagttggttgagg

SEQ ID NO: 34 - DNA, 30 bp, Artificial Sequence
  Description of Artificial Sequence Primer LW1385
gcataagcttccatggaacttcataacctg

SEQ ID NO: 35 - DNA, 30 bp, Artificial Sequence
  Description of Artificial Sequence Primer LW1386
gcatctcgagttacccccacagcgctgcag

SEQ ID NO: 36 - DNA, 29 bp, Artificial Sequence
  Description of Artificial Sequence Primer LW1329
gcatctcgagtcagcctaaggttatgttg

SEQ ID NO: 37 - DNA, 29 bp, Artificial Sequence
  Description of Artificial Sequence Primer LW1377
gcataagcttatgaacaccacagtgatgc

SEQ ID NO: 38 - DNA, 41 bp, Artificial Sequence
  Description of Artificial Sequence Primer LW1387
gagaaatatttttctaaaaaaacctgtttttgcaaaaacgg

SEQ ID NO: 39 - DNA, 41 bp, Artificial Sequence
  Description of Artificial Sequence Primer LW1388
ccgtttttgcaaaaacaggtttttttagaaaaatatttctc

SEQ ID NO: 40 - DNA, 30 bp, Artificial Sequence
  Description of Artificial Sequence Primer LW1314
gcatgaattcccaccttcatcatctacctc

SEQ ID NO: 41 - DNA, 29 bp, Artificial Sequence
  Description of Artificial Sequence Primer LW1315
gcatggatccgaagaccaaaaagacccag

SEQ ID NO: 42 - DNA, 30 bp, Artificial Sequence
  Description of Artificial Sequence Primer LW1326
gcatgaattcatgatggtggatcccaatgg

SEQ ID NO: 43 - DNA, 27 bp, Artificial Sequence
  Description of Artificial Sequence Primer LW1327
gcatctcgagcctagggctctgaagcg

SEQ ID NO: 44 - DNA, 42 bp, Artificial Sequence
  Description of Artificial Sequence Primer LW1415
ccatgtatatatttctttgcatgctttcaggcattgacatcc

SEQ ID NO: 45 - DNA, 42 bp, Artificial Sequence
  Description of Artificial Sequence Primer LW1416
ggatgtcaatgcctgaaagcatgcaaagaaatatatacatgg

SEQ ID NO: 46 - DNA, 30 bp, Artificial Sequence
  Description of Artificial Sequence Primer LW1308
gcatgaattcactcacttctcatctccttc

SEQ ID NO: 47 - DNA, 30 bp, Artificial Sequence
  Description of Artificial Sequence Primer LW1309
gcatggatccaatctcctttgtcttcactc

SEQ ID NO: 48 - DNA, 27 bp, Artificial Sequence
  Description of Artificial Sequence Primer LW1324
gatcggatccatggaaagcgagaacag

SEQ ID NO: 49 - DNA, 35 bp, Artificial Sequence
  Description of Artificial Sequence Primer LW1325
gatcctcgagtcaggctatgtgcttattaaacacc

SEQ ID NO: 50 - DNA, 29 bp, Artificial Sequence
  Description of Artificial Sequence Primer LW1306
gcatgaattcttctacttcatcatcctcc

SEQ ID NO: 51 - DNA, 28 bp, Artificial Sequence
  Description of Artificial Sequence Primer LW1307
gcatggatccaaaggccatcacaacaag

SEQ ID NO: 52 - DNA, 30 bp, Artificial Sequence
  Description of Artificial Sequence Primer GV599
ggcagaagaaggctattggtcttagacgag

SEQ ID NO: 53 - DNA, 22 bp, Artificial Sequence
  Description of Artificial Sequence Primer GV600
ctgaaacagcgcctcagctccc

SEQ ID NO: 54 - DNA, 27 bp, Artificial Sequence
  Description of Artificial Sequence Primer LW1482
agctatggcgaactatagccatgcagc

SEQ ID NO: 55 - DNA, 27 bp, Artificial Sequence
  Description of Artificial Sequence Primer LW148
agtcctcatataacacagtaaggttcc

SEQ ID NO: 56 - DNA, 28 bp, Artificial Sequence
  Description of Artificial Sequence Primer LW1310
gcatgaattcgcagaagaaggctattgg

SEQ ID NO: 57 - DNA, 29 bp, Artificial Sequence
  Description of Artificial Sequence Primer LW1311
gcatggatccgcagtaaagaagggttgtg

SEQ ID NO: 58 - DNA, 19 bp, Artificial Sequence
  Description of Artificial Sequence Primer LW1442
gccattctgtccacagaag

SEQ ID NO: 59 - DNA, 19 bp, Artificial Sequence
  Description of Artificial Sequence Primer LW1443
tcagttgctgttatggcac

SEQ ID NO: 60 - DNA, 24 bp, Artificial Sequence
  Description of Artificial Sequence Primer LW1440
aagcggatgtttagacctcttgtg

SEQ ID NO: 61 - DNA, 23 bp, Artificial Sequence
  Description of Artificial Sequence Primer LW1441
aacagtcatgaataggaattgag

SEQ ID NO: 62 - DNA, 32 bp, Artificial Sequence
  Description of Artificial Sequence Primer LW1472
gcatgaattctgccatgtcaatcatttctctc

SEQ ID NO: 63 - DNA, 31 bp, Artificial Sequence
  Description of Artificial Sequence Primer LW1473
gcatggatccgttctgcattttccaggtctc

SEQ ID NO: 64 - DNA, 29 bp, Artificial Sequence
  Description of Artificial Sequence Primer LW1411
gcatgaattctgccaaacatcatcctgac

SEQ ID NO: 65 - DNA, 29 bp, Artificial Sequence
  Description of Artificial Sequence Primer LW1412
gcatggatcctacacagccacaacaaccc

SEQ ID NO: 66 - DNA, 30 bp, Artificial Sequence
  Description of Artificial Sequence Primer LW1448
aagcggtaccatgttagccaacagctcctc

SEQ ID NO: 67 - DNA, 29 bp, Artificial Sequence
  Description of Artificial Sequence Primer LW1449
aagctctagatcagagggcggaatcctgg

SEQ ID NO: 68 - DNA, 43 bp, Artificial Sequence
  Description of Artificial Sequence Primer 217A
taggtcggtagtcaggacacgggagaacagaactgttggttga

SEQ ID NO: 69 - DNA, 52 bp, Artificial Sequence
  Description of Artificial Sequence Primer 217B
gcccctgtggcggtttagatccagaatgcccattttctgttccatctaacca

SEQ ID NO: 70 - DNA, 20 bp, Artificial Sequence
  Description of Artificial Sequence Primer LW1480
ggttctacctggacttatgg

SEQ ID NO: 71 - DNA, 20 bp, Artificial Sequence
  Description of Artificial Sequence Primer LW1481
taatgaatgagtaagtgccc

SEQ ID NO: 72 - DNA, 42 bp, Artificial Sequence
  Description of Artificial Sequence Primer CON103a
tttattaatattggaagggacaaactggagagcacagaacat

SEQ ID NO: 73 - DNA, 44 bp, Artificial Sequence
  Description of Artificial Sequence Primer CON103b
aaagccaccatggaagccatgccaaagatgatgctgggcaagaa

SEQ ID NO: 74 - DNA, 18 bp, Artificial Sequence
  Description of Artificial Sequence Primer 1332
tcctactgtcatgaaccc

SEQ ID NO: 75 - DNA, 18 bp, Artificial Sequence
  Description of Artificial Sequence Primer 1333
cagaagaagttgtccagc

(19) United States
(12) Patent Application Publication
(10) Pub. No.: US 2004/0091928 A1
(43) Pub. Date: May 13, 2004

(54) G protein-coupled receptors expressed in brain

(75) Inventors: Gabriel Vogeli, Seattle, WA (US); Linda S. Wood, Portage, MI (US); Kalpana Merchant, Portage, MI (US)

Correspondence Address: MARSHALL, GERSTEIN & BORUN LLP, 6300 SEARS TOWER, 233 S. WACKER DRIVE, CHICAGO, IL, 60606, US

(73) Assignee: PHARMACIA & UPJOHN COMPANY, Kalamazoo, MI

(21) Appl. No.: 10/692,605

(22) Filed: Oct. 24, 2003

Related U.S. Application Data
(63) Continuation of application No. 09/698,419, filed on Oct. 27, 2000.
(63) Continuation-in-part of application No. 09/481,794, filed on Jan. 12, 2000, now abandoned.
(63) Continuation of application No. 09/454,399, filed on Dec. 3, 1999, now abandoned.
(63) Continuation of application No. 09/429,517, filed on Oct. 28, 1999, now abandoned.
(63) Continuation of application No. 09/429,555, filed on Oct. 28, 1999, now abandoned.
(63) Continuation of application No. 09/429,676, filed on Oct. 28, 1999, now abandoned.
(63) Continuation of application No. 09/429,695, filed on Oct. 28, 1999, now abandoned.
(63) Continuation of application No. 09/428,114, filed on Oct. 27, 1999, now abandoned.
(63) Continuation of application No. 09/428,020, filed on Oct. 27, 1999, now abandoned.
(63) Continuation of application No. 09/427,859, filed on Oct. 27, 1999, now abandoned.
(63) Continuation of application No. 09/427,653, filed on Oct. 27, 1999, now abandoned.

(51) Int. Cl. C12Q 1/68; C07H 21/04; C07K 14/705

(52) U.S. Cl. 435/6; 435/69.1; 435/320.1; 435/325; 530/350; 536/23.5

(57) ABSTRACT

The present invention provides genes encoding heretofore unknown G protein-coupled receptors, constructs and recombinant host cells incorporating the genes; the GPCR polypeptides encoded by the genes; antibodies to the polypeptides; and methods of making and using all of the foregoing.

### RELATED APPLICATIONS

This patent application is a continuation-in-part of the following U.S. patent applications: Ser. No. 09/481,794 filed Jan. 12, 2000; Ser. No. 09/454,399 filed Dec. 3, 1999; Ser. Nos. 09/429,517, 09/429,555, 09/429,676, 09/429,695 filed Oct. 28, 1999; and Ser. Nos. 09/428,114, 09/428,020, 09/427,859 and 09/427,653 filed Oct. 27, 1999. All these application are incorporated herein by reference.

### FIELD OF THE INVENTION

The present invention relates generally to the fields of genetics and cellular and molecular biology. More particularly, the invention relates to a novel G protein-coupled seven transmembrane receptor polynucleotide and polypeptide sequences that are expressed in the brain.

### DESCRIPTION OF RELATED ART

Humans and other life forms are comprised of living cells. Among the mechanisms through which the cells of an organism communicate with each other and obtain information and stimuli from their environment is through cell membrane receptor molecules expressed on the cell surface. Many such receptors have been identified, characterized, and sometimes classified into major receptor superfamilies based on structural motifs and signal transduction features. Such families include (but are not limited to) ligand-gated ion channel receptors, voltage-dependent ion channel receptors, receptor tyrosine kinases, receptor protein tyrosine phosphatases, and G protein-coupled receptors. The receptors are a first essential link for translating an extracellular signal into a cellular physiological response.

The G protein-coupled receptors (GPCR) form a vast superfamily of cell surface receptors which are characterized by an amino-terminal extracellular domain, a carboxyl-terminal intracellular domain, and a serpentine structure that passes through the cell membrane seven times. Hence, such receptors are sometimes also referred to as seven transmembrane (7TM) receptors. These seven transmembrane domains define three extracellular loops and three intracellular loops, in addition to the amino- and carboxyl-terminal domains. The extracellular portions of the receptor have a role in recognizing and binding one or more extracellular binding partners (ligands), whereas the intracellular portions have a role in recognizing and communicating with downstream effector molecules.

The G protein-coupled receptors bind a variety of ligands including calcium ions, hormones, chemokines, neuropeptides, neurotransmitters, nucleotides, lipids, odorants, and even photons, and are important in the normal (and sometimes the aberrant) function of many cell types. [See generally A. D. Strosberg, Eur. J Biochem., 196: 1-10 (1991) and S. K. Bohm et al., Biochem J., 322: 1-18 (1997).] When a specific ligand binds to its corresponding receptor, the ligand stimulates the receptor to activate a specific heterotrimeric guanine-nucleotide-binding regulatory protein (G-protein) that is coupled to the intracellular portion of the receptor. The G protein in turn transmits a signal to an effector molecule within the cell, by either stimulating or inhibiting the activity of that effector molecule. These effector molecules include adenylate cyclase, phospholipases, and ion channels. Adenylate cyclase and phospholipases are enzymes that are involved in the production of the second messenger molecules cAMP, inositol triphosphate and diacyglycerol. It is through this sequence of events that an extracellular ligand stimuli exerts intracellular changes through a G protein-coupled receptor. Each such receptor has its own characteristic primary structure, expression pattern, ligand-binding profile, and intracellular effector system.

Because of the vital role of G protein-coupled receptors in the communication between cells and their environment, such receptors are attractive targets for therapeutic intervention, and many drugs have been registered which are directed towards activating or antagonizing such receptors. For receptors having a known ligand, the identification of agonists or antagonists may be sought specifically for enhancing or inhibiting the action of the ligand. Some G protein-coupled receptors have roles in disease pathogenesis (e.g., certain chemokine receptors that act as HIV co-receptors and may have a role in AIDS pathogenesis), and are attractive targets for therapeutic intervention even in the absence of knowledge of the natural ligand of the receptor. Other receptors are attractive targets for therapeutic intervention by virtue of their expression pattern in tissues or cell types that are attractive targets for therapeutic intervention. Examples of this latter category of receptors include receptors expressed in immune cells, for targeting to enhance immune responses to fight pathogens or cancer or inhibit autoimmune responses; and receptors expressed in the brain or other neurons, for targeting to treat schizophrenia, depression, bipolar disease, or other neurological disorders. This latter category of receptor is also useful as a marker for identifying and/or purifying (e.g., via fluorescence activated cell sorting) cellular subtypes that express the receptor. Unfortunately, only a limited number of G protein receptors from the central nervous system (CNS) are known. A need exists for identifying the existence and structure of such G protein-coupled receptors.

### SUMMARY OF THE INVENTION.

The present invention addresses one or more of the needs identified above in that it provides purified polynucleotides encoding heretofore unknown G protein-coupled receptors (GPCR); constructs and recombinant host cells incorporating the polynucleotides; GPCR polypeptides encoded by the polynucleotides; antibodies to the polypeptides; and methods of making and using all of the foregoing. As set forth in detail herein, the GPCR polypeptides described herein are expressed in the brain, providing a therapeutic indication for GPCR polypeptides and binding partners to treat diseases associated with this tissue.

The invention provides purified and isolated GPCR seven transmembrane receptor polypeptides comprising any one of the amino acid sequences set forth in SEQ ID NOS: 2, 4, 6, 8, 10, 12, 14, 16, 18 or 20, or a fragment thereof comprising an epitope specific to the seven transmembrane receptor. By “epitope specific to” is meant a portion of the receptor that is recognizable by an antibody that is specific for that seven transmembrane receptor, as defined in detail below.

One preferred embodiment comprises a purified and isolated polypeptide designated CON193, comprising the complete amino acid sequence set forth in SEQ ID NO: 2. This amino acid sequence was deduced from a polynucleotide sequence encoding CON193 (SEQ ID NO:1), as set forth below:

| | |
|---|---|
| ntggttgttg gaccattaaa atgcattatg gaatttttaa aagttggggg agagggagac | 60 |
| agtaaaaata acctatattt tctcttgttt tttttttttt aactctagga aagcccagac | 120 |
| aaattttgag ctatttcata acctaccaga cttatc atg cta aca ctg aat aaa | 174 |
| Met Leu Thr Leu Asn Lys | |
| 1 5 | |
| aca gac cta ata cca gct tca ttt att ctg aat gga gtc cca gga ctg | 222 |
| Thr Asp Leu Ile Pro Ala Ser Phe Ile Leu Asn Gly Val Pro Gly Leu | |
| 10 15 20 | |
| gaa gac aca caa ctc tgg att tcc ttc cca ttc tgc tct atg tat gtt | 270 |
| Glu Asp Thr Gln Leu Trp Ile Ser Phe Pro Phe Cys Ser Met Tyr Val | |
| 25 30 35 | |
| gtg gct atg gta ggg aat tgt gga ctc ctc tac ctc att cac tat gag | 318 |
| Val Ala Met Val Gly Asn Cys Gly Leu Leu Tyr Leu Ile His Tyr Glu | |
| 40 45 50 | |
| gat gcc ctg cac aaa ccc atg tac tac ttc ttg gcc atg ctt tcc ttt | 366 |
| Asp Ala Leu His Lys Pro Met Tyr Tyr Phe Leu Ala Met Leu Ser Phe | |
| 55 60 65 70 | |
| act gac ctt gtt atg tgc tct agt aca atc cct aaa gcc ctc tgc atc | 414 |
| Thr Asp Leu Val Met Cys Ser Ser Thr Ile Pro Lys Ala Leu Cys Ile | |
| 75 80 85 | |
| ttc tgg ttt cat ctc aag gac att gga ttt gat gaa tgc ctt gtc cag | 462 |
| Phe Trp Phe His Leu Lys Asp Ile Gly Phe Asp Glu Cys Leu Val Gln | |
| 90 95 100 | |
| atg ttc ttc atc cac acc ttc aca ggg atg gag tct ggg gtg ctt atg | 510 |
| Met Phe Phe Ile His Thr Phe Thr Gly Met Glu Ser Gly Val Leu Met | |
| 105 110 115 | |
| ctt atg gcc ctg gat cgc tat gtg gcc atc tgc tac ccc tta cgc tat | 558 |
| Leu Met Ala Leu Asp Arg Tyr Val Ala Ile Cys Tyr Pro Leu Arg Tyr | |
| 120 125 130 | |
| tca act atc ctc acc aat cct gta att gca aag gtt ggg act gcc acc | 606 |
| Ser Thr Ile Leu Thr Asn Pro Val Ile Ala Lys Val Gly Thr Ala Thr | |
| 135 140 145 150 | |
| ttc ctg aga ggg gta tta ctc att att ccc ttt act ttc ctc acc aag | 654 |
| Phe Leu Arg Gly Val Leu Leu Ile Ile Pro Phe Thr Phe Leu Thr Lys | |
| 155 160 165 | |
| cgc ctg ccc tcc tgc aga ggc aat ata ctt ccc cat acc tac tgt gac | 702 |
| Arg Leu Pro Ser Cys Arg Gly Asn Ile Leu Pro His Thr Tyr Cys Asp | |
| 170 175 180 | |
| cac atg tct gta gcc aaa ttg tcc tgt ggt aat gtc aag gtc aat gcc | 750 |
| His Met Ser Val Ala Lys Leu Ser Cys Gly Asn Val Lys Val Asn Ala | |
| 185 190 195 | |
| atc tat ggt ctg atg gtt gcc ctc ctg att ggg ggc ttt gac ata ctg | 798 |
| Ile Tyr Gly Leu Met Val Ala Leu Leu Ile Gly Gly Phe Asp Ile Leu | |
| 200 205 210 | |
| tgt atc acc atc tcc tat acc atg att ctc cgg gca gtg gtc agc ctc | 846 |
| Cys Ile Thr Ile Ser Tyr Thr Met Ile Leu Arg Ala Val Val Ser Leu | |
| 215 220 225 230 | |
| tcc tca gca gat gct cgg cag aag gcc ttt aat acc tgc act gcc cac | 894 |
| Ser Ser Ala Asp Ala Arg Gln Lys Ala Phe Asn Thr Cys Thr Ala His | |
| 235 240 245 | |
| att tgt gcc att gtt ttc tcc tat act cca gct ttc ttc tcc ttc ttt | 942 |
| Ile Cys Ala Ile Val Phe Ser Tyr Thr Pro Ala Phe Phe Ser Phe Phe | |
| 250 255 260 | |
| tcc cac cgc ttt ggg gaa cac ata atc ccc cct tct tgc cac atc att | 990 |
| Ser His Arg Phe Gly Glu His Ile Ile Pro Pro Ser Cys His Ile Ile | |
| 265 270 275 | |
| gta gcc aat att tat ctg ctc cta cca ccc act atg aac cct att gtc | 1038 |
| Val Ala Asn Ile Tyr Leu Leu Leu Pro Pro Thr Met Asn Pro Ile Val | |
| 280 285 290 | |
| tat ggg gtg aaa acc aaa cag ata cga gac tgt gtc ata agg atc ctt | 1086 |
| Tyr Gly Val Lys Thr Lys Gln Ile Arg Asp Cys Val Ile Arg Ile Leu | |
| 295 300 305 310 | |
| tca ggt tct aag gat acc aaa tcc tac agc atg tga atgaacactt | 1132 |
| Ser Gly Ser Lys Asp Thr Lys Ser Tyr Ser Met | |
| 315 320 | |
| gccaggagtg agaagagaag gaaagaatta cttctatttg cctcttatgc aggagttcat | 1192 |
| aaaatctttc tggaagtact gtattgatca caaaatggag tttgntgact ggtgcattc | 1252 |
| caataagtac cttgggaatc tnacatcact ggaaggccca ccacatttct ataaat | 1308 |

Another preferred embodiment comprises a purified and isolated polypeptide designated CON166, comprising the complete amino acid sequence set forth in SEQ ID NO: 4. This amino acid sequence was deduced from a polynucleotide sequence encoding CON166 (SEQ ID NO: 3), as set forth below:

| | |
|---|---|
| atg gat gaa aca gga aat ctg aca gta tct tct gcc aca tgc cat gac | 48 |
| Met Asp Glu Thr Gly Asn Leu Thr Val Ser Ser Ala Thr Cys His Asp | |
| 1 5 10 15 | |
| act att gat gac ttc cgc aat caa gtg tat tcc acc ttg tac tct atg | 96 |
| Thr Ile Asp Asp Phe Arg Asn Gln Val Tyr Ser Thr Leu Tyr Ser Met | |
| 20 25 30 | |
| atc tct gtt gta ggc ttc ttt ggc aat ggc ttt gtg ctc tat gtc ctc | 144 |
| Ile Ser Val Val Gly Phe Phe Gly Asn Gly Phe Val Leu Tyr Val Leu | |
| 35 40 45 | |
| ata aaa acc tat cac aag aag tca gcc ttc caa gta tac atg att aat | 192 |
| Ile Lys Thr Tyr His Lys Lys Ser Ala Phe Gln Val Tyr Met Ile Asn | |
| 50 55 60 | |
| tta gca gta gca gat cta ctt tgt gtg tgc aca ctg cct ctc cgt gtg | 240 |
| Leu Ala Val Ala Asp Leu Leu Cys Val Cys Thr Leu Pro Leu Arg Val | |
| 65 70 75 80 | |
| gtc tat tat gtt cac aaa ggc att tgg ctc ttt ggt gac ttc ttg tgc | 288 |
| Val Tyr Tyr Val His Lys Gly Ile Trp Leu Phe Gly Asp Phe Leu Cys | |
| 85 90 95 | |
| cgc ctc agc acc tat gct ttg tat gtc aac ctc tat tgt agc atc ttc | 336 |
| Arg Leu Ser Thr Tyr Ala Leu Tyr Val Asn Leu Tyr Cys Ser Ile Phe | |
| 100 105 110 | |
| ttt atg aca gcc atg agc ttt ttc cgg tgc att gca att gtt ttt cca | 384 |
| Phe Met Thr Ala Met Ser Phe Phe Arg Cys Ile Ala Ile Val Phe Pro | |
| 115 120 125 | |
| gtc cag aac att aat ttg gtt aca cag aaa aaa gcc agg ttt gtg tgt | 432 |
| Val Gln Asn Ile Asn Leu Val Thr Gln Lys Lys Ala Arg Phe Val Cys | |
| 130 135 140 | |
| gta ggt att tgg att ttt gtg att ttg acc agt tct cca ttt cta atg | 480 |
| Val Gly Ile Trp Ile Phe Val Ile Leu Thr Ser Ser Pro Phe Leu Met | |
| 145 150 155 160 | |
| gcc aaa cca caa aaa gat gag aaa aat aat acc aag tgc ttt gag ccc | 528 |
| Ala Lys Pro Gln Lys Asp Glu Lys Asn Asn Thr Lys Cys Phe Glu Pro | |
| 165 170 175 | |
| cca caa gac aat caa act aaa aat cat gtt ttg gtc ttg cat tat gtg | 576 |
| Pro Gln Asp Asn Gln Thr Lys Asn His Val Leu Val Leu His Tyr Val | |
| 180 185 190 | |
| tca ttg ttt gtt ggc ttt atc atc cct ttt gtt att ata att gtc tgt | 624 |
| Ser Leu Phe Val Gly Phe Ile Ile Pro Phe Val Ile Ile Ile Val Cys | |
| 195 200 205 | |
| tac aca atg atc att ttg acc tta cta aaa aaa tca atg aaa aaa aat | 672 |
| Tyr Thr Met Ile Ile Leu Thr Leu Leu Lys Lys Ser Met Lys Lys Asn | |
| 210 215 220 | |
| ctg tca agt cat aaa aag gct ata gga atg atc atg gtc gtg acc gct | 720 |
| Leu Ser Ser His Lys Lys Ala Ile Gly Met Ile Met Val Val Thr Ala | |
| 225 230 235 240 | |
| gcc ttt tta gtc agt ttc atg cca tat cat att caa cgt acc att cac | 768 |
| Ala Phe Leu Val Ser Phe Met Pro Tyr His Ile Gln Arg Thr Ile His | |
| 245 250 255 | |
| ctt cat ttt tta cac aat gaa act aaa ccc tgt gat tct gtc ctt aga | 816 |
| Leu His Phe Leu His Asn Glu Thr Lys Pro Cys Asp Ser Val Leu Arg | |
| 260 265 270 | |
| atg cag aag tcc gtg gtc ata acc ttg tct ctg gct gca tcc aat tgt | 864 |
| Met Gln Lys Ser Val Val Ile Thr Leu Ser Leu Ala Ala Ser Asn Cys | |
| 275 280 285 | |
| tgc ttt gac cct ctc cta tat ttc ttt tct ggg ggt aac ttt agg aaa | 912 |
| Cys Phe Asp Pro Leu Leu Tyr Phe Phe Ser Gly Gly Asn Phe Arg Lys | |
| 290 295 300 | |
| agg ctg tct aca ttt aga aag cat tct ttg tcc agc gtg act tat gta | 960 |
| Arg Leu Ser Thr Phe Arg Lys His Ser Leu Ser Ser Val Thr Tyr Val | |
| 305 310 315 320 | |
| ccc aga aag aag gcc tct ttg cca gaa aaa gga gaa gaa ata tgt aaa | 1008 |
| Pro Arg Lys Lys Ala Ser Leu Pro Glu Lys Gly Glu Glu Ile Cys Lys | |
| 325 330 335 | |
| gta tag 1014 | |
| Val | |

Still another preferred embodiment comprises a purified and isolated polypeptide designated CON103, comprising the complete amino acid sequence set forth in SEQ ID NO: 6. This amino acid sequence was deduced from a polynucleotide sequence encoding CON103 (SEQ ID NO: 5), as set forth below:

| | |
|---|---|
| ggggcctact tcaccgtgta cccggacttg ggaccatcac agacttcaga accatcagga | 60 |
| acctgggagc aactgaaagc tgaactacag tgggctttca gacacacagc aggctgcgga | 120 |
| gcacaaatag gactggttcc ctccaggcca ccagcagggc ggtggaggtc ttcactgact | 180 |
| ccctgcctac ctctcaggac aatgtccttt tggctccaca gtccctgaag ccagagctgg | 240 |
| tgggggcagg gaggcagcca ccagcctcta tatgtagtgg aggagggggt gtccagggag | 300 |
| ggctgcatga tcctgagagc ccccacctca cccggctgga ctatcctccc acttcagggt | 360 |
| ttctctgggc ttccatcttg cccctgctga gccctgcttc ctcctctacc agcagcacaa | 420 |
| cccccaggct gggctcagag acctcatgtg gtgggatcac tcagtacccc gaggcggagg | 480 |
| gaaggaggga gggctgcagg gttccccttg gcctgcaaac aggaacacag ggtgtttctc | 540 |
| agtggctgcg agaatgctga tgaaaacccc aggatgttgt gtcaccgtgg tggccagctg | 600 |
| atagtgccaa tcatcccact ttgccctgag cactcctgca ggggtagaag actccagaac | 660 |
| cttctctcag gcccatggcc caagcagccc atg gaa ctt cat aac ctg agc tct | 714 |
| Met Glu Leu His Asn Leu Ser Ser | |
| 1 5 | |
| cca tct ccc tct ctc tcc tcc tct gtt ctc cct ccc tcc ttc tct ccc | 762 |
| Pro Ser Pro Ser Leu Ser Ser Ser Val Leu Pro Pro Ser Phe Ser Pro | |
| 10 15 20 | |
| tca ccc tcc tct gct ccc tct gcc ttt acc act gtg ggg ggg tcc tct | 810 |
| Ser Pro Ser Ser Ala Pro Ser Ala Phe Thr Thr Val Gly Gly Ser Ser | |
| 25 30 35 40 | |
| gga ggg ccc tgc cac ccc acc tct tcc tcg ctg gtg tct gcc ttc ctg | 858 |
| Gly Gly Pro Cys His Pro Thr Ser Ser Ser Leu Val Ser Ala Phe Leu | |
| 45 50 55 | |
| gca cca atc ctg gcc ctg gag ttt gtc ctg ggc ctg gtg ggg aac agt | 906 |
| Ala Pro Ile Leu Ala Leu Glu Phe Val Leu Gly Leu Val Gly Asn Ser | |
| 60 65 70 | |
| ttg gcc ctc ttc atc ttc tgc atc cac acg cgg ccc tgg acc tcc aac | 954 |
| Leu Ala Leu Phe Ile Phe Cys Ile His Thr Arg Pro Trp Thr Ser Asn | |
| 75 80 85 | |
| acg gtg ttc ctg gtc agc ctg gtg gcc gct gac ttc ctc ctg atc agc | 1002 |
| Thr Val Phe Leu Val Ser Leu Val Ala Ala Asp Phe Leu Leu Ile Ser | |
| 90 95 100 | |
| aac ctg ccc ctc cgc gtg gac tac tac ctc ctc cat gag acc tgg cgc | 1050 |
| Asn Leu Pro Leu Arg Val Asp Tyr Leu Leu His Glu Thr Trp Arg | |
| 105 110 115 120 | |
| ttt ggg gct gct gcc tgc aaa gtc aac ctc ttc atg ctg tcc acc aac | 1098 |
| Phe Gly Ala Ala Ala Cys Lys Val Asn Leu Phe Met Leu Ser Thr Asn | |
| 125 130 135 | |
| cgc acg gcc agc gtt gtc ttc ctc aca gcc atc gca ctc aac cgc tac | 1146 |
| Arg Thr Ala Ser Val Val Phe Leu Thr Ala Ile Ala Leu Asn Arg Tyr | |
| 140 145 150 | |
| ctg aag gtg gtg cag ccc cac cac gtg ctg agc cgt gct tcc gtg ggg | 1194 |
| Leu Lys Val Val Gln Pro His His Val Leu Ser Arg Ala Ser Val Gly | |
| 155 160 165 | |
| gca gct gcc cgg gtg gcc ggg gga ctc tgg gtg ggc atc ctg ctc ctc | 1242 |
| Ala Ala Ala Arg Val Ala Gly Gly Leu Trp Val Gly Ile Leu Leu Leu | |
| 170 175 180 | |
| aac ggg cac ctg ctc ctg agc acc ttc tcc ggc ccc tcc tgc ctc agc | 1290 |
| Asn Gly His Leu Leu Leu Ser Thr Phe Ser Gly Pro Ser Cys Leu Ser | |
| 185 190 195 200 | |
| tac agg gtg ggc acg aag ccc tcg gcc tcg ctc cgc tgg cac cag gca | 1338 |
| Tyr Arg Val Gly Thr Lys Pro Ser Ala Ser Leu Arg Trp His Gln Ala | |
| 205 210 215 | |
| ctg tac ctg ctg gag ttc ttc ctg cca ctg gcg ctc atc ctc ttt gct | 1386 |
| Leu Tyr Leu Leu Glu Phe Phe Leu Pro Leu Ala Leu Ile Leu Phe Ala | |
| 220 225 230 | |
| att gtg agc att ggg ctc acc atc cgg aac cgt ggt ctg ggc ggg cag | 1434 |
| Ile Val Ser Ile Gly Leu Thr Ile Arg Asn Arg Gly Leu Gly Gly Gln | |
| 235 240 245 | |
| gca ggc ccg cag agg gcc atg cgt gtg ctg gcc atg gtg gtg gcc gtc | 1482 |
| Ala Gly Pro Gln Arg Ala Met Arg Val Leu Ala Met Val Val Ala Val | |
| 250 255 260 | |
| tac acc atc tgc ttc ttg ccc agc atc atc ttt ggc atg gct tcc atg | 1530 |
| Tyr Thr Ile Cys Phe Leu Pro Ser Ile Ile Phe Gly Met Ala Ser Met | |
| 265 270 275 280 | |
| gtg gct ttc tgg ctg tcc gcc tgc cga tcc ctg gac ctc tgc aca cag | 1578 |
| Val Ala Phe Trp Leu Ser Ala Cys Arg Ser Leu Asp Leu Cys Thr Gln | |
| 285 290 295 | |
| ctc ttc cat ggc tcc ctg gcc ttc acc tac ctc aac agt gtc ctg gac | 1626 |
| Leu Phe His Gly Ser Leu Ala Phe Thr Tyr Leu Asn Ser Val Leu Asp | |
| 300 305 310 | |
| ccc gtg ctc tac tgc ttc tct agc ccc aac ttc ctc cac cag agc cgg | 1674 |
| Pro Val Leu Tyr Cys Phe Ser Ser Pro Asn Phe Leu His Gln Ser Arg | |
| 315 320 325 | |
| gcc ttg ctg ggc ctc acg cgg ggc cgg cag ggc cca gtg agc gac gag | 1722 |
| Ala Leu Leu Gly Leu Thr Arg Gly Arg Gln Gly Pro Val Ser Asp Glu | |
| 330 335 340 | |
| agc tcc tac caa ccc tcc agg cag tgg cgc tac cgg gag gcc tct agg | 1770 |
| Ser Ser Tyr Gln Pro Ser Arg Gln Trp Arg Tyr Arg Glu Ala Ser Arg | |
| 345 350 355 360 | |
| aag gcg gag gcc ata ggg aag ctg aaa gtg cag ggc gag gtc tct ctg | 1818 |
| Lys Ala Glu Ala Ile Gly Lys Leu Lys Val Gln Gly Glu Val Ser Leu | |
| 365 370 375 | |
| gaa aag gaa ggc tcc tcc cag ggc tga gggccagctg cagggctgca 1865 | |
| Glu Lys Glu Gly Ser Ser Gln Gly | |
| 380 385 | |
| gcgctgtggg ggtaagggct gccgcgctct ggcctggagg gacaaggcca gcacacggtg | 1925 |
| cctcaaccaa ctggacaagg gatggcggca gaccaggggc caggccaaag cactggcagg | 1985 |
| actcatgtgg gtggcaggga gagaaaccca cctaggcctc tcagtgtgtc caggatggca | 2045 |
| ttcccagaat gcaggggaga gcaggatgcc gggtggagga gacaggcaag gtgccgttgg | 2105 |
| cacaccagct cagacagggg cctgcgcagc tgcaggggac agacgccaat cactgtcaca | 2165 |
| gcagagtcac cttagaaatt ggacagctgc atgttctgtg ctctccagtt tgtcccttcc | 2225 |
| aatattaata aacttccctt ttaaatatat ttatttgcag accaatatct gtctttaatt | 2285 |
| ctaacctggg actgtcagta ggcgtcaaag tgagcgcccc agtgaaggaa ccttggagag | 2345 |
| agtgggagca ttcccagcct tccaggggga ctcgtcttcc agactttgga gcccgcatgt | 2405 |
| ctgaagcaga ctctttcttg gtag 2429 | |

Another preferred embodiment comprises a purified and isolated polypeptide designated CON203, comprising the complete amino acid sequence set forth in SEQ ID NO: 8. This amino acid sequence was deduced from a polynucleotide sequence encoding CON203 (SEQ ID NO: 7), as set forth below:

| | |
|---|---|
| ttgaatttag gtgacactat agaagagcta tgacgtcgca tgcacgcgta cgtaagctcg | 60 |
| gaattcggct cgagctgaac taatgactgc cgccataaga agacagagag aactgagtat | 120 |
| cctcccaaag gtgacactgg aagca atg aac acc aca gtg atg caa ggc ttc | 172 |
| Met Asn Thr Thr Val Met Gln Gly Phe | |
| 1 5 | |
| aac aga tct gag cgg tgc ccc aga gac act cgg ata gta cag ctg gta | 220 |
| Asn Arg Ser Glu Arg Cys Pro Arg Asp Thr Arg Ile Val Gln Leu Val | |
| 10 15 20 25 | |
| ttc cca gcc ctc tac aca gtg gtt ttc ttg acc ggc atc ctg ctg aat | 268 |
| Phe Pro Ala Leu Tyr Thr Val Val Phe Leu Thr Gly Ile Leu Leu Asn | |
| 30 35 40 | |
| act ttg gct ctg tgg gtg ttt gtt cac atc ccc agc tcc tcc acc ttc | 316 |
| Thr Leu Ala Leu Trp Val Phe Val His Ile Pro Ser Ser Ser Thr Phe | |
| 45 50 55 | |
| atc atc tac ctc aaa aac act ttg gtg gcc gac ttg ata atg aca ctc | 364 |
| Ile Ile Tyr Leu Lys Asn Thr Leu Val Ala Asp Leu Ile Met Thr Leu | |
| 60 65 70 | |
| atg ctt cct ttc aaa atc ctc tct gac tca cac ctg gca ccc tgg cag | 412 |
| Met Leu Pro Phe Lys Ile Leu Ser Asp Ser His Leu Ala Pro Trp Gln | |
| 75 80 85 | |
| ctc aga gct ttt gtg tgt cgt ttt tct tcg gtg ata ttt tat gag acc | 460 |
| Leu Arg Ala Phe Val Cys Arg Phe Ser Ser Val Ile Phe Tyr Glu Thr | |
| 90 95 100 105 | |
| atg tat gtg ggc atc gtg ctg tta ggg ctc ata gcc ttt gac aga ttc | 508 |
| Met Tyr Val Gly Ile Val Leu Leu Gly Leu Ile Ala Phe Asp Arg Phe | |
| 110 115 120 | |
| ctc aag atc atc aga cct ttg aga aat att ttt cta aaa aaa cct gtt | 556 |
| Leu Lys Ile Ile Arg Pro Leu Arg Asn Ile Phe Leu Lys Lys Pro Val | |
| 125 130 135 | |
| ttt gca aaa acg gtc tca atc ttc atc tgg gtc ttt ttg gtc ttc atc | 604 |
| Phe Ala Lys Thr Val Ser Ile Phe Ile Trp Val Phe Leu Val Phe Ile | |
| 140 145 150 | |
| tcc ctg cca aat atg atc ttg agc aac aag gaa gca aca cca tcg tct | 652 |
| Ser Leu Pro Asn Met Ile Leu Ser Asn Lys Glu Ala Thr Pro Ser Ser | |
| 155 160 165 | |
| gtg aaa aag tgt gct tcc tta aag ggg cct ctg ggg ctg aaa tgg cat | 700 |
| Val Lys Lys Cys Ala Ser Leu Lys Gly Pro Leu Gly Leu Lys Trp His | |
| 170 175 180 185 | |
| caa atg gta aat aac ata tgc cag ttt att ttc tgg act ggt ttt atc | 748 |
| Gln Met Val Asn Asn Ile Cys Gln Phe Ile Phe Trp Thr Gly Phe Ile | |
| 190 195 200 | |
| cta atg ctt gtg ttt tat gtg gtt att gca aaa aaa gta tat gat tct | 796 |
| Leu Met Leu Val Phe Tyr Val Val Ile Ala Lys Lys Val Tyr Asp Ser | |
| 205 210 215 | |
| tat aga aag tcc aaa agt aag gac aga aaa aac aac aaa aag ctg gaa | 844 |
| Tyr Arg Lys Ser Lys Ser Lys Asp Arg Lys Asn Asn Lys Lys Leu Glu | |
| 220 225 230 | |
| ggc aaa gta ttt gtt gtc gtg gct gtc ttc ttt gtg tgt ttt gct cca | 892 |
| Gly Lys Val Phe Val Val Val Ala Val Phe Phe Val Cys Phe Ala Pro | |
| 235 240 245 | |
| ttt cat ttt gcc aga gtt cca tat act cac agt caa acc aac aat aag | 940 |
| Phe His Phe Ala Arg Val Pro Tyr Thr His Ser Gln Thr Asn Asn Lys | |
| 250 255 260 265 | |
| act gac tgt aga ctg caa aat caa ctg ttt att gct aaa gaa aca act | 988 |
| Thr Asp Cys Arg Leu Gln Asn Gln Leu Phe Ile Ala Lys Glu Thr Thr | |
| 270 275 280 | |
| ctc ttt ttg gca gca act aac att tgt atg gat ccc tta ata tac ata | 1036 |
| Leu Phe Leu Ala Ala Thr Asn Ile Cys Met Asp Pro Leu Ile Tyr Ile | |
| 285 290 295 | |
| ttc tta tgt aaa aaa ttc aca gaa aag cta cca tgt atg caa ggg aga | 1084 |
| Phe Leu Cys Lys Lys Phe Thr Glu Lys Leu Pro Cys Met Gln Gly Arg | |
| 300 305 310 | |
| aag acc aca gca tca agc caa gaa aat cat agc agt cag aca gac aac | 1132 |
| Lys Thr Thr Ala Ser Ser Gln Glu Asn His Ser Ser Gln Thr Asp Asn | |
| 315 320 325 | |
| ata acc tta ggc tga caactgtaca tagggttaac ttctatttat tgatgagact | 1187 |
| Ile Thr Leu Gly | |
| 330 | |
| tccgtagata atgtggaaat caaatttaac caagaaaaaa agattggaac aaatgctctc | 1247 |
| ttacatttta tttatcctgg tgtccaggaa aagattatat taaatttaaa tccacataga | 1307 |
| tctattcata agctgaatga accattacct aagagaatgc aacaggatac caatggccac | 1367 |
| tagaggcata ttccttcttc tttttttttt gttaaatttc aagagcattc actttacatt | 1427 |

tggaaagact aaggggaacg gttatcctac aaacctccct tcaacacctt ttacatt 1484

Another preferred embodiment comprises a purified and isolated polypeptide designated CON198, comprising the complete amino acid sequence set forth in SEQ ID NO: 10. This amino acid sequence was deduced from a polynucleotide sequence encoding CON198 (SEQ ID NO: 9), as set forth below:

| | |
|---|---|
| atg atg gtg gat ccc aat ggc aat gaa tcc agt gct aca tac ttc atc | 48 |
| Met Met Val Asp Pro Asn Gly Asn Glu Ser Ser Ala Thr Tyr Phe Ile | |
| 1 5 10 15 | |
| cta ata ggc ctc cct ggt tta gaa gag gct cag ttc tgg ttg gcc ttc | 96 |
| Leu Ile Gly Leu Pro Gly Leu Glu Glu Ala Gln Phe Trp Leu Ala Phe | |
| 20 25 30 | |
| cca ttg tgc tcc ctc tac ctt att gct gtg cta ggt aac ttg aca atc | 144 |
| Pro Leu Cys Ser Leu Tyr Leu Ile Ala Val Leu Gly Asn Leu Thr Ile | |
| 35 40 45 | |
| atc tac att gtg cgg act gag cac agc ctg cat gag ccc atg tat ata | 192 |
| Ile Tyr Ile Val Arg Thr Glu His Ser Leu His Glu Pro Met Tyr Ile | |
| 50 55 60 | |
| ttt ctt tgc atg ctt tca ggc att gac atc ctc atc tcc acc tca tcc | 240 |
| Phe Leu Cys Met Leu Ser Gly Ile Asp Ile Leu Ile Ser Thr Ser Ser | |
| 65 70 75 80 | |
| atg ccc aaa atg ctg gcc atc ttc tgg ttc aat tcc act acc atc cag | 288 |
| Met Pro Lys Met Leu Ala Ile Phe Trp Phe Asn Ser Thr Thr Ile Gln | |
| 85 90 95 | |
| ttt gat gct tgt ctg cta cag atg ttt gcc atc cac tcc tta tct ggc | 336 |
| Phe Asp Ala Cys Leu Leu Gln Met Phe Ala Ile His Ser Leu Ser Gly | |
| 100 105 110 | |
| atg gaa tcc aca gtg ctg ctg gcc atg gct ttt gac cgc tat gtg gcc | 384 |
| Met Glu Ser Thr Val Leu Leu Ala Met Ala Phe Asp Arg Tyr Val Ala | |
| 115 120 125 | |
| atc tgt cac cca ctg cgc cat gcc aca gta ctt acg ttg cct cgt gtc | 432 |
| Ile Cys His Pro Leu Arg His Ala Thr Val Leu Thr Leu Pro Arg Val | |
| 130 135 140 | |
| acc aaa att ggt gtg gct gct gtg gtg cgg ggg gct gca ctg atg gca | 480 |
| Thr Lys Ile Gly Val Ala Ala Val Val Arg Gly Ala Ala Leu Met Ala | |
| 145 150 155 160 | |
| ccc ctt cct gtc ttc atc aag cag ctg ccc ttc tgc cgc tcc aat atc | 528 |
| Pro Leu Pro Val Phe Ile Lys Gln Leu Pro Phe Cys Arg Ser Asn Ile | |
| 165 170 175 | |
| ctt tcc cat tcc tac tgc cta cac caa gat gtc atg aag ctg gcc tgt | 576 |
| Leu Ser His Ser Tyr Cys Leu His Gln Asp Val Met Lys Leu Ala Cys | |
| 180 185 190 | |
| gat gat atc cgg gtc aat gtc gtc tat ggc ctt atc gtc atc atc tcc | 624 |
| Asp Asp Ile Arg Val Asn Val Val Tyr Gly Leu Ile Val Ile Ile Ser | |
| 195 200 205 | |
| gcc att ggc ctg gac tca ctt ctc atc tcc ttc tca tat ctg ctt att | 672 |
| Ala Ile Gly Leu Asp Ser Leu Leu Ile Ser Phe Ser Tyr Leu Leu Ile | |
| 210 215 220 | |
| ctt aag act gtg ttg ggc ttg aca cgt gaa gcc cag gcc aag gca ttt | 720 |
| Leu Lys Thr Val Leu Gly Leu Thr Arg Glu Ala Gln Ala Lys Ala Phe | |
| 225 230 235 240 | |
| ggc act tgc gtc tct cat gtg tgt gct gtg ttc ata ttc tat gta cct | 768 |
| Gly Thr Cys Val Ser His Val Cys Ala Val Phe Ile Phe Tyr Val Pro | |
| 245 250 255 | |
| ttc att gga ttg tcc atg gtg cat cgc ttt agc aag cgg cgt gac tct | 816 |
| Phe Ile Gly Leu Ser Met Val His Arg Phe Ser Lys Arg Arg Asp Ser | |
| 260 265 270 | |
| ccg ctg ccc gtc atc ttg gcc aat atc tat ctg ctg gtt cct cct gtg | 864 |
| Pro Leu Pro Val Ile Leu Ala Asn Ile Tyr Leu Leu Val Pro Pro Val | |
| 275 280 285 | |
| ctc aac cca att gtc tat gga gtg aag aca aag gag att cga cag cgc | 912 |
| Leu Asn Pro Ile Val Tyr Gly Val Lys Thr Lys Glu Ile Arg Gln Arg | |
| 290 295 300 | |
| atc ctt cga ctt ttc cat gtg gcc aca cac gct tca gag ccc tag | 957 |
| Ile Leu Arg Leu Phe His Val Ala Thr His Ala Ser Glu Pro | |
| 305 310 315 | |

It will be appreciated that SEQ ID NO: 10 contains methionine residues at positions 1 and 2. Translation of the relevant mRNA sequences may occur beginning from either or both methionines, which can be determined for a particular cell source by purifying expressed CON198 protein and performing amino-terminal sequencing thereon. CON198 polypeptides beginning at either Met₁, or Met₂, of SEQ ID NO: 10 are intended a polypeptides of the invention.

Another preferred embodiment comprises a purified and isolated polypeptide designated CON197, comprising the complete amino acid sequence set forth in SEQ ID NO: 12. This amino acid sequence was deduced from a polynucleotide sequence encoding CON197 (SEQ ID NO: 11), as set forth below:

```
1
ATGGAAAGCGAGAACAGAAGAGTGATAAGAGAATTCATCCTCCTTGGTCTGACCCAGTCTCAAGATATT
  M E S E N R R V I R E F I L L G L T Q S Q D I
70
CAGCTCCTGGTCTTTGTGCTAGTTTTAATATTCTACTTCATCATCCTCCCTGGAAATTTTCTCATTATT
  Q L L V F V L V L I F Y F I I L P G N F L I I
139
TTCACCATAAAGTCAGACCCTGGGCTCACAGCCCCCCTCTATTTCTTTCTGGGCAACTTGGCCTTCCTG
  F T I K S D P G L T A P L Y F F L G N L A F L
208
GATGCATCCTACTCCTTCATTGTGGCTCCCCGGATGTTGGTGGACTTCCTCTCTGCGAAGAAGATAATC
  D A S Y S F I V A P R M L V D F L S A K K I I
277
TCCTACAGAGGCTGCATCACTCAGCTCTTTTTCTTGCACTTCCTTGGAGGAGGGGAGGGATTACTCCTT
  S Y R G C I T Q L F F L H F L G G G E G L L L
346
GTTGTGATGGCCTTTGACCGCTACATCGCCATCTGCCGGCCTCTGCACTATCCTACTGTCATGAACCCT
  V V M A F D R Y I A I C R P L H Y P T V M N P
415
AGAACCTGCTATGCAATGATGTTGGCTCTGTGGCTTGGGGGTTTTGTCCACTCCATTATCCAGGTGGTC
  R T C Y A M M L A L W L G G F V H S I I Q V V
484
CTCATCCTCCGCTTGCCTTTTTGTGGCCCAAACCAGCTGGACAACTTCTTCTGTGATGTCCCACAGGTC
  L I L R L P F C G P N Q L D N F F C D V P Q V
553
ATCAAGCTGGCCTGCACCGACACATTTGTGGTGGAGCTTCTGATGGTCTTCAACAGTGGCCTGATGACA
  I K L A C T D T F V V E L L M V F N S G L M T
622
CTCCTGTGCTTTCTGGGGCTTCTGGCCTCCTATGCAGTCATTCTTTGTCGCATACGAGGGTCTTCTTCT
  L L C F L G L L A S Y A V I L C R I R G S S S
691
GAGGCAAAAAACAAGGCCATGTCCACGTGCATCACCCATATCATTGTTATATTCTTCATGTTTGGACCT
  E A K N K A M S T C I T H I I V I F F M F G P
760
GGCATCTTCATCTACACGCGCCCCTTCAGGGCTTTCCCAGCTGACAAGGTGGTTTCTCTCTTCCACACA
  G I F I Y T R P F R A F P A D K V V S L F H T
829
GTGATTTTTCCTTTGTTGAATCCTGTCATTTATACCCTTCGCAACCAGGAAGTGAAAGCTTCCATGAAA
  V I F P L L N P V I Y T L R N Q E V K A S M K
898
AAGGTGTTTAATAAGCACATAGCCTGAAAAAGGGCGCAAAAAAAAAAAGAATAAAAATAGACTGTAGAA
  K V F N K H I A *
967
TTTTTAAAAAAAAAAAAAAAAAAAAAAAA
```

Another preferred embodiment comprises a purified and isolated polypeptide designated CON202, comprising the complete amino acid sequence set forth in SEQ ID NO: 14. This amino acid sequence was deduced from a polynucleotide sequence encoding CON202 (SEQ ID NO: 13), as set forth below:

```
1
TGCTTCCCCATAAGGTAACAGCTTTGTTAGCNCTGTCTGACATCATTGCTTGTTNACTTAAGAACTGAT
70
AGGTNTTTTTTTTTTTTTTTTTTCAGATATTCTGATGGCAAAACAAGTGGAAGAAAAGAGGAAGCATGA
139
CTGCAGATCAGATCAGTTCTCTTTGTGGATTATATTTTCAGTAAAATGTATGGATCTATCTTTTCCTTG
208
TTCTTATATCTAGATCATGAGACTTGACTGAGGCTGTATCCTTATCCTCCATCCATCTATGGCGAACTA
                                                                                                                      M A N Y
277
TAGCCATGCAGCTGACAACATTTTGCAAAATCTCTCGCCTCTAACAGCCTTTCTGAAACTGACTTCCTT
    S H A A D N I L Q N L S P L T A F L K L T S L
346
GGGTTTCATAATAGGAGTCAGCGTGGTGGGCAACCTCCTGATCTCCATTTTGCTAGTGAAAGATAAGAC
    G F I I G V S V V G N L L I S I L L V K D K T
415
CTTGCATAGAGCACCTTACTACTTCCTGTTGGATCTTTGCTGTTCAGATATCCTCAGATCTGCAATTTG
    L H R A P Y Y F L L D L C C S D I L R S A I C
484
TTTCCCATTTGTGTTCAACTCTGTCAAAAATGGTTCTACCTGGACTTATGGGACTCTGACTTGCAAAGT
    F P F V F N S V K N G S T W T Y G T L T C K V
553
GATTGCCTTTCTGGGGGTTTTGTCCTGTTTCCACACTGCTTTCATGCTCTTCTGCATCAGTGTCACCAG
    I A F L G V L S C F H T A F M L F C I S V T R
622
ATATTTAGCTATCGCCCATCACCGCTTCTATACAAAGAGGCTGACCTTTTGGACGTGTCTGGCTGTGAT
    Y L A I A H H R F Y T K R L T F W T C L A V I
691
CTGTATGGTGTGGACTCTGTCTGTGGCCATGGCATTTCCCCCGGTTTTAGACGTGGGCACTTACTCATT
    C M V W T L S V A M A F P P V L D V G T Y S F
760
CATTAGGGAGGAAGATCAATGCACCTTCCAACACCGCTCCTTCAGGGCTAATGATTCCTTAGAATTTAT
    I R E E D Q C T F Q H R S F R A N D S L G F M
829
GCTGCTTCTTGCTCTCATCCTCCTAGCCACACAGCTTGTCTACCTCAAGCTGATATTTTTCGTCCACGA
    L L L A L I L L A T Q L V Y L K L I F F V H D
898
TCGAAGAAAAATGAAGCCAGTCCAGTTTGTAGCAGCAGTCAGCCAGAACTGGACTTTTCATGGTCCTGG
    R R K M K P V Q F V A A V S Q N W T F H G P G
967
AGCCAGTGGCCAGGCAGCTGCCAATTGGCTAGCAGGATTTGGAAGGGGTCCCACACCACCCACCTTGCT
    A S G Q A A A N W L A G F G R G P T P P T L L
1036
GGGCATCAGGCAAAATGCAAACACCACAGGCAGAAGAAGGCTATTGGTCTTAGACGAGTTCAAAATGGA
    G I R Q N A N T T G R R R L L V L D E F K M E
1105
GAAAAGAATCAGCAGAATGTTCTATATAATGACTTTTCTGTTTCTAACCTTGTGGGGCCCCTACCTGGT
    K R I S R M F Y I M T F L F L T L W G P Y L V
1174
GGCCTGTTATTGGAGAGTTTTTGCAAGAGGGCCTGTAGTACCAGGGGGATTTCTAACAGCTGCTGTCTG
    A C Y W R V F A R G P V V P G G F L T A A V W
1243
GATGAGTTTTGCCCAAGCAGGAATCAATCCTTTTGTCTGCATTTTCTCAAACAGGGAGCTGAGGCGCTG
    M S F A Q A G I N P F V C I F S N R E L R R C
1312
TTTCAGCACAACCCTTCTTTACTGCAGAAAATCCAGGTTACCAAGGGAACCTTACTGTGTTATATGAGG
    F S T T L L Y C R K S R L P R E P Y C V I
```

Still another preferred embodiment comprises a purified and isolated polypeptide designated CON222, comprising the complete amino acid sequence set forth in SEQ ID NO: 16. This amino acid sequence was deduced from a polynucleotide sequence encoding CON222 (SEQ ID NO: 15), as set forth below:

| | | |
|---|---|---|
| 1 | ATGTTTAGACCTCTTGTGAATCTCTCTCACATATATTTTAAGAAATTCCAGTACTGTGGGTATGCA | |
| | M F R P L V N L S H I Y F K K F Q Y C G Y A | |
| 67 | CCACATGTTCGCAGCTGTAAACCAAACACTGATGGAATTTCATCTCTAGAGAATCTCTTGGCAAGC | |
| | P H V R S C K P N T D G I S S L E N L L A S | |
| 133 | ATTATTCAGAGAGTATTTGTCTGGGTTGTATCTGCAGTTACCTGCTTTGGAAACATTTTTGTCATT | |
| | I I Q R V F V W V V S A V T C F G N I F V I | |
| 199 | TGCATGCGACCTTATATCAGGTCTGAGAACAAGCTGTATGCCATGTCAATCATTTCTCTCTGCTGT | |
| | C M R P Y I R S E N K L Y A M S I I S L C C | |
| 265 | GCCGACTGCTTAATGGGAATATATTTATTCGTGATCGGAGGCTTTGACCTAAAGTTTCGTGGAGAA | |
| | A D C L M G I Y L F V I G G F D L K F R G E | |
| 331 | TACAATAAGCATGCGCAGCTGTGGATGGAGAGTACTCATTGTCAGCTTGTAGGATCTTTGGCCATT | |
| | Y N K H A Q L W M E S T H C Q L V G S L A I | |
| 397 | CTGTCCACAGAAGTATCAGTTTTACTGTTAACATTTCTGACATTGGAAAAATACATCTGCATTGTC | |
| | L S T E V S V L L L T F L T L E K Y I C I V | |
| 463 | TATCCTTTTAGATGTGTGAGACCTGGAAAATGCAGAACAATTACAGTTCTGATTCTCATTTGGATT | |
| | Y P F R C V R P G K C R T I T V L I L I W I | |
| 529 | ACTGGTTTTATAGTGGCTTTCATTCCATTGAGCAATAAGGAATTTTTCAAAAACTACTATGGCACC | |
| | T G F I V A F I P L S N K E F F K N Y Y G T | |
| 595 | AATGGAGTATGCTTCCCTCTTCATTCAGAAGATACAGAAAGTATTGGAGCCCAGATTTATTCAGTG | |
| | N G V C F P L H S E D T E S I G A Q I Y S V | |
| 661 | GCAATTTTTCTTGGTATTAATTTGGCCGCATTTATCATCATAGTTTTTTCCTATGGAAGCATGTTT | |
| | A I F L G I N L A A F I I I V F S Y G S M F | |
| 727 | TATAGTGTTCATCAAAGTGCCATAACAGCAACTGAAATACGGAATCAAGTTAAAAAAGAGATGATC | |
| | Y S V H Q S A I T A T E I R N Q V K K E M I | |
| 793 | CTTGCCAAACGTTTTTTCTTTATAGTATTTACTGATGCATTATGCTGGATACCCATTTTTGTAGTG | |
| | L A K R F F F I V F T D A L C W I P I F V V | |
| 859 | AAATTTCTTTCACTGCTTCAGGTAGAAATACCAGGTACCATAACCTCTTGGGTAGTGATTTTTATT | |
| | K F L S L L Q V E I P G T I T S W V V I F I | |
| 925 | CTGCCCATTAACAGTGCTTTGAACCCAATTCTCTATACTCTGACCACAAGACCATTTAAAGAAATG | |
| | L P I N S A L N P I L Y T L T T R P F K E M | |
| 991 | ATTCATCGGTTTTGGTATAACTACAGACAAAGAAAATCTATGGACAGCAAAGGTCAGAAAACATAT | |
| | I H R F W Y N Y R Q R K S M D S K G Q K T Y | |
| 1057 | GCTCCATCATTCATCTGGGTGGAAATGTGGCCACTGCAGGAGATGCCACCTGAGTTAATGAAGCCG | |
| | A P S F I W V E M W P L Q E M P P E L M K P | 1123 |
| | GACCTTTTCACATACCCCTGTGAAATGTCACTGATTTCTCAATCAACGAGACTCAATTCCTATTCA | |
| | D L F T Y P C E M S L I S Q S T R L N S Y S | |
| 1189 | TGA 1191 | |
| * | | |

Another preferred embodiment comprises a purified and isolated polypeptide designated CON215, comprising the complete amino acid sequence set forth in SEQ ID NO: 18. This amino acid sequence was deduced from a polynucleotide sequence encoding CON215 (SEQ ID NO: 17), as set forth below:

| | |
|---|---|
| atg ggg ttc aac ttg acg ctt gca aaa tta cca aat aac gag ctg cac | 48 |
| Met Gly Phe Asn Leu Thr Leu Ala Lys Leu Pro Asn Asn Glu Leu His | |
| 1 5 10 15 | |
| ggc caa gag agt cac aat tca ggc aac agg agc gac ggg cca gga aag | 96 |
| Gly Gln Glu Ser His Asn Ser Gly Asn Arg Ser Asp Gly Pro Gly Lys | |
| 20 25 30 | |
| aac acc acc ctt cac aat gaa ttt gac aca att gtc ttg cca gtg ctt | 144 |
| Asn Thr Thr Leu His Asn Glu Phe Asp Thr Ile Val Leu Pro Val Leu | |
| 35 40 45 | |
| tat ctc att ata ttt gtg gca agc atc ttg ctg aat ggt tta gca gtg | 192 |
| Tyr Leu Ile Ile Phe Val Ala Ser Ile Leu Leu Asn Gly Leu Ala Val | |
| 50 55 60 | |
| tgg atc ttc ttc cac att agg aat aaa acc agc ttc ata ttc tat ctc | 240 |
| Trp Ile Phe Phe His Ile Arg Asn Lys Thr Ser Phe Ile Phe Tyr Leu | |
| 65 70 75 80 | |
| aaa aac ata gtg gtt gca gac ctc ata atg acg ctg aca ttt cca ttt | 288 |
| Lys Asn Ile Val Val Ala Asp Leu Ile Met Thr Leu Thr Phe Pro Phe | |
| 85 90 95 | |
| cga ata gtc cat gat gca gga ttt gga cct tgg tac ttc aag ttt att | 336 |
| Arg Ile Val His Asp Ala Gly Phe Gly Pro Trp Tyr Phe Lys Phe Ile | |
| 100 105 110 | |
| ctc tgc aga tac act tca gtt ttg ttt tat gca aac atg tat act tcc | 384 |
| Leu Cys Arg Tyr Thr Ser Val Leu Phe Tyr Ala Asn Met Tyr Thr Ser | |
| 115 120 125 | |
| atc gtg ttc ctt ggg ctg ata agc att gat cgc tat ctg aag gtg gtc | 432 |
| Ile Val Phe Leu Gly Leu Ile Ser Ile Asp Arg Tyr Leu Lys Val Val | |
| 130 135 140 | |
| aag cca ttt ggg gac tct cgg atg tac agc ata acc ttc acg aag gtt | 480 |
| Lys Pro Phe Gly Asp Ser Arg Met Tyr Ser Ile Thr Phe Thr Lys Val | |
| 145 150 155 160 | |
| tta tct gtt tgt gtt tgg gtg atc atg gct gtt ttg tct ttg cca aac | 528 |
| Leu Ser Val Cys Val Trp Val Ile Met Ala Val Leu Ser Leu Pro Asn | |
| 165 170 175 | |
| atc atc ctg aca aat ggt cag cca aca gag gac aat atc cat gac tgc | 576 |
| Ile Ile Leu Thr Asn Gly Gln Pro Thr Glu Asp Asn Ile His Asp Cys | |
| 180 185 190 | |
| tca aaa ctt aaa agt cct ttg ggg gtc aaa tgg cat acg gca gtc acc | 624 |
| Ser Lys Leu Lys Ser Pro Leu Gly Val Lys Trp His Thr Ala Val Thr | |
| 195 200 205 | |
| tat gtg aac agc tgc ttg ttt gtg gcc gtg ctg gtg att ctg atc gga | 672 |
| Tyr Val Asn Ser Cys Leu Phe Val Ala Val Leu Val Ile Leu Ile Gly | |
| 210 215 220 | |
| tgt tac ata gcc ata tcc agg tac atc cac aaa tcc agc agg caa ttc | 720 |
| Cys Tyr Ile Ala Ile Ser Arg Tyr Ile His Lys Ser Ser Arg Gln Phe | |
| 225 230 235 240 | |
| ata agt cag tca agc cga aag cga aaa cat aac cag agc atc agg gtt | 768 |
| Ile Ser Gln Ser Ser Arg Lys Arg Lys His Asn Gln Ser Ile Arg Val | |
| 245 250 255 | |
| gtt gtg gct gtg ttt ttt acc tgc ttt cta cca tat cac ttg tgc aga | 816 |
| Val Val Ala Val Phe Phe Thr Cys Phe Leu Pro Tyr His Leu Cys Arg | |
| 260 265 270 | |
| att cct ttt act ttt agt cac tta gac agg ctt tta gat gaa tct gca | 864 |
| Ile Pro Phe Thr Phe Ser His Leu Asp Arg Leu Leu Asp Glu Ser Ala | |
| 275 280 285 | |
| caa aaa atc cta tat tac tgc aaa gaa att aca ctt ttc ttg tct gcg | 912 |
| Gln Lys Ile Leu Tyr Tyr Cys Lys Glu Ile Thr Leu Phe Leu Ser Ala | |
| 290 295 300 | |
| tgt aat gtt tgc ctg gat cca ata att tac ttt ttc atg tgt agg tca | 960 |
| Cys Asn Val Cys Leu Asp Pro Ile Ile Tyr Phe Phe Met Cys Arg Ser | |
| 305 310 315 320 | |
| ttt tca aga agg ctg ttc aaa aaa tca aat atc aga acc agg agt gaa | 1008 |
| Phe Ser Arg Arg Leu Phe Lys Lys Ser Asn Ile Arg Thr Arg Ser Glu | |
| 325 330 335 | |
| agc atc aga tca ctg caa agt gtg aga aga tcg gaa gtt ctc ata tat | 1056 |
| Ser Ile Arg Ser Leu Gln Ser Val Arg Arg Ser Glu Val Leu Ile Tyr | |
| 340 345 350 | |
| tat gat tat act gat gtg tag | 1077 |
| Tyr Asp Tyr Thr Asp Val | |
| 355 | |

Another preferred embodiment comprises a purified and isolated polypeptide designated CON217, comprising the complete amino acid sequence set forth in SEQ ID NO: 20. This amino acid sequence was deduced from a polynucleotide sequence encoding CON217 (SEQ ID NO: 19), as set forth below:

| | |
|---|---|
| −41 | C ATGGCATCCC CAGCCTAGCT CCCAATCCCA CTTTGGCACG |
| 1 | ATGTTAGCCAACAGCTCCTCAACCAACAGTTCTGTTCTCCCGTGTCCTGACTACCGACCTACCCAC |
| | M L A N S S S T N S S V L P C P D Y R P T H |
| 67 | CGCCTGCACTTGGTGGTCTACAGCTTGGTGCTGGCTGCCGGGCTCCCCCTCAACGCGCTAGCCCTC |
| | R L H L V V Y S L V L A A G L P L N A L A L |
| 133 | TGGGTCTTCCTGCGCGCGCTGCGCGTGCACTCGGTGGTGAGCGTGTACATGTGTAACCTGGCGGCC |
| | W V F L R A L R V H S V V S V Y M C N L A A |
| 199 | AGCGACCTGCTCTTCACCCTCTCGCTGCCCGTTCGTCTCTCCTACTACGCACTGCACCACTGGCCC |
| | S D L L F T L S L P V R L S Y Y A L H H W P |
| 265 | TTCCCCGACCTCCTGTGCCAGACGACGGGCGCCATCTTCCAGATGAACATGTACGGCAGCTGCATC |
| | F P D L L C Q T T G A I F Q M N M Y G S C I |
| 331 | TTCCTGATGCTCATCAACGTGGACCGCTACGCCGCCATCGTGCACCCGCTGCGACTGCGCCACCTG |
| | F L M L I N V D R Y A A I V H P L R L R H L |
| 397 | CGGCGGCCCCGCGTGGCGCGGCTGCTCTGCCTGGGCGTGTGGGCGCTCATCCTGGTGTTTGCCGTG |
| | R R P R V A R L L C L G V W A L I L V F A V |
| 463 | CCCGCCGCCCGCGTGCACAGGCCCTCGCGTTGCCGCTACCGGGACCTCGAGGTGCGCCTATGCTTC |
| | P A A R V H R P S R C R Y R D L E V R L C F |
| 529 | GAGAGCTTCAGCGACGAGCTGTGGAAAGGCAGGCTGCTGCCCCTCGTGCTGCTGGCCGAGGCGCTG |
| | E S F S D E L W K G R L L P L V L L A E A L |
| 595 | GGCTTCCTGCTGCCCCTGGCGGCGGTGGTCTACTCGTCGGGCCGAGTCTTCTGGACGCTGGCGCGC |
| | G F L L P L A A V V Y S S G R V F W T L A R |
| 661 | CCCGACGCCACGCAGAGCCAGCGGCGGCGGAAGACCGTGCGCCTCCTGCTGGCTAACCTCGTCATC |
| | P D A T Q S Q R R R K T V R L L L A N L V I |
| 727 | TTCCTGCTGTGCTTCGTGCCCTACAACAGCACGCTGGCGGTCTACGGGCTGCTGCGGAGCAAGCTG |
| | F L L C F V P Y N S T L A V Y G L L R S K L |
| 793 | GTGGCGGCCAGCGTGCCTGCCCGCGATCGCGTGCGCGGGGTGCTGATGGTGATGGTGCTGCTGGCC |
| | V A A S V P A R D R V R G V L M V M V L L A |
| 959 | GGCGCCAACTGCGTGCTGGACCCGCTGGTGTACTACTTTAGCGCCGAGGGCTTCCGCAACACCCTG |
| | G A N C V L D P L V Y Y F S A E G F R N T L |
| 925 | CGCGGCCTGGGCACTCCGCACCGGGCCAGGACCTCGGCCACCAACGGGACGCGGGCGGCGCTCGCG |
| | R G L G T P H R A R T S A T N G T R A A L A |
| 991 | CAATCCGAAAGGTCCGCCGTCACCACCGACGCCACCAGGCCGGATGCCGCCAGTCAGGGGCTGCTC |
| | Q S E R S A V T T D A T R P D A A S Q G L L |
| 1057 | CGACCCTCCGACTCCCACTCTCTGTCTTCCTTCACACAGTGTCCCCAGGATTCCGCCCTCTGAACA |
| | R P S D S H S L S S F T Q C P Q D S A L * |
| 1123 | CACATGCCAT TGCGCTGTCC GTGCCCGACT CCCAACGCCT CTCGTTCTGG GAGGCTTACA |
| 1183 | GGGTGTACAC ACAAGAAGGT GGGCTGGGCA CTTGGACCTT TGGGTGGCAA TTCCAGCTTA |
| 1243 | GCAACGCAGA AGAGTACAAA GTGTGGAAGC CAGGGCCCAG GGAAGGCAGT GCTGCTGGAA |
| 1303 | ATGGCTTCTT TAAACTGTGA GCACGCAGAG CACCCCTTCT CCAGCGGTGG GAAGTGATGC |
| 1363 | AGAGAGCCCA CCCGTGCAGA GGGCAGAAGA GGACGAAATG CCTTTGGGTG GGCAGGGCAT |
| 1423 | TAAACTGCTA AAAGCTGGTT AGATGGAACA GAAAATGGGC ATTCTGGATC TAAACCGCCA |
| 1483 | CAGGGGCCTG AGAGCTGAAG AGCACCAGGT TTGGTGGACA AAGCTACTGA GATGCCTGTT |
| 1543 | CATCTGCTGA CTTCTGTCTA GGCTCATGGA TGCCACCCCC TTTCATTTCG GCCTAGGCTT |
| 1603 | CCCCTGCTCA CCACTGAGGC CTAATACAAG AGTTCCTATG GACAGAACTA CATTCTTTCT |
| 1663 | CGCATAGTGA CTTGTGACAA TTTAGACTTG GCATCCAGCA TGGGATAGTT GGGGCAAGGC |
| 1723 | AAAACTAACT TAGAGTTTCC CCCTCAACAA CATCCAAGTC CAAACCCTTT TTAGGTTATC |
| 1783 | CTTTCTTCCA TCACATCCCC TTTTCCAGGC CTCCTCCATT TTAGGTCCTT AATATTCTTT |
| 1843 | CTTTTTCTCT CTCTCTCGTT TCTCTCTTCT CTCTCCTCTC CTCTCCTCTC TCTTCTCCTC |
| 1903 | TTCTCTCTCT CTCCCTCTCT CTCCTTTGTC CAGAGTAAGG ATAAAATTCT TTCTACTAAA |
| 1963 | GCACTGGTTC TCAAACTTTT TGGTCTCAGA CCCCACTCTT AGAAATTGAG GATCTCAAAG |
| 2023 | AGCTTTGCTT ATATTTTGTT CTTTTGATAC TTACCATACT AGAAATTAAA GCGAATACAT |
| 2083 | TTTTAAAATA AATACACATG CACACATTAC ATTAGCCATG GGAGCAATAA TGTCACCACA |
| 2143 | CACACTTCAT GAAGCCTCTG GAAAACTCTA CAGTATACTT GTGAGAGAAT GAGAGTGAAA |
| 2203 | GGGACAAATA ACATCTGTGT AGCAGTATTA TGAAAATAGC TTGACCTTGT GGACTTCCTC |
| 2263 | AGAGGGTTGG TCCCTGGATC ACACTTTGAG AACCATACTT GTCCTGAAGT ATTGGAGTTC |
| 2323 | ATGTCTAACT TCTTCCCAGG GCATTATGTA CAGTGCTTTT TATTACTGTG GGGAGAGGGC |
| 2383 | AGTGCTAAAT AAATTAATCA CTACTGATAA AAAAAAAAAA AAAAAAAAAA AAAAAAA |

Although SEQ ID NOS: 2, 4, 6, 8, 10, 12, 14, 16, 18, and 20 provide for particular human sequences, the invention is intended to include within its scope other human allelic variants; non-human mammalian forms of GPCR polypeptides, and other vertebrate forms of GPCR polypeptides.

It will be appreciated that extracellular epitopes are particularly useful for generating and screening for antibodies and other binding compounds that bind to receptors such as GPCR polypeptides. Thus, in another preferred embodiment, the invention provides a purified and isolated polypeptide comprising at least one extracellular domain of a GPCR polypeptide of the invention. By “extracellular domain”, is it meant the amino terminal extracellular domain or an extracellular loop that spans two membrane domains.

A purified and isolated polypeptide comprising the N-terminal extracellular domain of GPCR polypeptides of the invention is highly preferred. Also preferred is a purified and isolated polypeptide comprising a GPCR seven transmembrane receptor fragment selected from the group consisting of the N-terminal extracellular domain of GPCR polypeptides of the invention, transmembrane domains of GPCR polypeptides of the invention, extracellular loops connecting transmembrane domains of GPCR polypeptides of the invention. intracellular loops connecting transmembrane domains of GPCR polypeptides of the invention, the C-terminal cytoplasmic domain of GPCR polypeptides, and fusions thereof. Such fragments may be continuous portions of the native receptor. However, it will also be appreciated that knowledge of the GPCR gene and protein sequences as provided herein permits recombining of various domains that are not contiguous in the native protein.

In another embodiment, the invention provides purified and isolated polynucleotides (e.g., cDNA, genomic DNA, synthetic DNA, RNA, or combinations thereof, single or double stranded) that comprise a nucleotide sequence encoding an amino acid sequence of the polypeptides of the invention. Another embodiment provides a purified and isolated polynucleotide encoding the amino acid sequence of the polypeptide of the invention fused to a heterologous tag amino acid sequence. Such polynucleotides are useful for recombinantly expressing the receptor and also for detecting expression of the receptor in cells (e.g., using Northern hybridization and in situ hybridization assays, and Western studies). Polynucleotides encoding polypeptides of the invention also are useful to design antisense and other molecules for the suppression of GPCR polypeptides expression in a cultured cell or animal (for therapeutic purposes or to provide a model for diseases characterized by aberrant GPCR polypeptide expression). Such polynucleotides are also useful to design antisense and other molecules for the suppression of GPCR polypeptide expression in a cultured cell or tissue or in an animal, for therapeutic purposes or to provide a model for diseases characterized by aberrant GPCR polypeptide expression. Specifically excluded from the definition of polynucleotides of the invention are entire isolated chromosomes of native host cells. A preferred polynucleotide set forth in any one of the SEQ ID NOS: 1, 3, 5, 7, 9, 11, 13, 15, 17, and 19 corresponds to a naturally occurring GPCR sequence. It will be appreciated that numerous other sequences exist that also encode GPCR polypeptides having the amino acid sequence set out in SEQ ID NOS: 2, 4, 6, 8. 10, 12, 14, 16, 18 and 20 due to the well-known degeneracy of the universal genetic code. All such sequences represent polynucleotides of the invention.

The invention also provides a purified and isolated polynucleotide comprising a nucleotide sequence that encodes a mammalian seven transmembrane receptor, wherein the polynucleotide hybridizes to a nucleotide sequence set forth in any one of SEQ ID NOS: 1, 3, 5, 7, 9, 11, 13, 15, 17, or 19 or the non-coding strand complementary thereto, under the following hybridization conditions:

(a) hybridization for 16 hours at 42° C. in a hybridization solution comprising 50% formamide, 1% SDS, 1 M NaCl, 10% Dextran sulphate; and

(b) washing 2 times for 30 minutes at 60° C. in a wash solution comprising 0.1% SSC, 1% SDS. Polynucleotides that encode a human allelic variant are highly preferred.

A highly preferred polynucleotide of the invention comprises the sequence set forth in SEQ ID NO: 1, which comprises a human CON193 encoding DNA sequence:

| | |
|---|---|
| ntggttgttg gaccattaaa atgcattatg gaatttttaa aagttggggg agagggagac | 60 |
| agtaaaaata acctatattt tctcttgttt tttttttttt aactctagga aagcccagac | 120 |
| aaattttgag ctatttcata acctaccaga cttatcatgc taacactgaa taaaacagac | 180 |
| ctaataccag cttcatttat tctgaatgga gtcccaggac tggaagacac acaactctgg | 240 |
| atttccttcc cattctgctc tatgtatgtt gtggctatgg tagggaattg tggactcctc | 300 |
| tacctcattc actatgagga tgccctgcac aaacccatgt actacttctt ggccatgctt | 360 |
| tcctttactg accttgttat gtgctctagt acaatcccta aagccctctg catcttctgg | 420 |
| tttcatctca aggacattgg atttgatgaa tgccttgtcc agatgttctt catccacacc | 480 |
| ttcacaggga tggagtctgg ggtgcttatg cttatggccc tggatcgcta tgtggccatc | 540 |
| tgctacccct tacgctattc aactatcctc accaatcctg taattgcaaa ggttgggact | 600 |
| gccaccttcc tgagaggggt attactcatt attcccttta ctttcctcac caagcgcctg | 660 |
| ccctcctgca gaggcaatat acttccccat acctactgtg accacatgtc tgtagccaaa | 720 |
| ttgtcctgtg gtaatgtcaa ggtcaatgcc atctatggtc tgatggttgc cctcctgatt | 780 |
| gggggctttg acatactgtg tatcaccatc tcctatacca tgattctccg ggcagtggtc | 840 |
| agcctctcct cagcagatgc tcggcagaag gcctttaata cctgcactgc ccacatttgt | 900 |
| gccattgttt tctcctatac tccagctttc ttctccttct tttcccaccg ctttggggaa | 960 |
| cacataatcc ccccttcttg ccacatcatt gtagccaata tttatctgct cctaccaccc | 1020 |
| actatgaacc ctattgtcta tggggtgaaa accaaacaga tacgagactg tgtcataagg | 1080 |
| atcctttcag gttctaagga taccaaatcc tacagcatgt gaatgaacac ttgccaggag | 1140 |
| tgagaagaga aggaaagaat tacttctatt tgcctcttat gcaggagttc ataaaatctt | 1200 |
| tctggaagta ctgtattgat cacaaaatgg agtttgntga ctggtgcatt ctcaataagt | 1260 |
| accttgggaa tctnacatca ctggaaggcc caccacattt ctataaat 1308 | |

Also preferred is a polynucleotide comprising nucleotides 157-1119 of SEQ ID NO: 1, which represent the portion of SEQ ID NO: 1 that encodes CON193 amino acids.

Another highly preferred polynucleotide of the invention comprises the sequence set forth in SEQ ID NO: 3, which comprises a human CON166 encoding DNA sequence:

| | |
|---|---|
| atggatgaaa caggaaatct gacagtatct tctgccacat gccatgacac tattgatgac | 60 |
| ttccgcaatc aagtgtattc caccttgtac tctatgatct ctgttgtagg cttctttggc | 120 |
| aatggctttg tgctctatgt cctcataaaa acctatcaca agaagtcagc cttccaagta | 180 |
| tacatgatta atttagcagt agcagatcta ctttgtgtgt gcacactgcc tctccgtgtg | 240 |
| gtctattatg ttcacaaagg catttggctc tttggtgact tcttgtgccg cctcagcacc | 300 |
| tatgctttgt atgtcaacct ctattgtagc atcttcttta tgacagccat gagctttttc | 360 |
| cggtgcattg caattgtttt tccagtccag aacattaatt tggttacaca gaaaaaagcc | 420 |
| aggtttgtgt gtgtaggtat ttggattttt gtgattttga ccagttctcc atttctaatg | 480 |
| gccaaaccac aaaaagatga gaaaaataat accaagtgct ttgagccccc acaagacaat | 540 |
| caaactaaaa atcatgtttt ggtcttgcat tatgtgtcat tgtttgttgg ctttatcatc | 600 |
| ccttttgtta ttataattgt ctgttacaca atgatcattt tgaccttact aaaaaaatca | 660 |
| atgaaaaaaa atctgtcaag tcataaaaag gctataggaa tgatcatggt cgtgaccgct | 720 |
| gcctttttag tcagtttcat gccatatcat attcaacgta ccattcacct tcatttttta | 780 |
| cacaatgaaa ctaaaccctg tgattctgtc cttagaatgc agaagtccgt ggtcataacc | 840 |
| ttgtctctgg ctgcatccaa ttgttgcttt gaccctctcc tatatttctt ttctgggggt | 900 |
| aactttagga aaaggctgtc tacatttaga aagcattctt tgtccagcgt gacttatgta | 960 |
| cccagaaaga aggcctcttt gccagaaaaa ggagaagaaa tatgtaaagt atag 1014 | |

The final three nucleotides of this sequence represent a stop codon.

Still another highly preferred polynucleotide of the invention comprises the sequence set forth in SEQ ID NO: 5, which comprises a human CON103 encoding DNA sequence:

| | |
|---|---|
| ggggcctact tcaccgtgta cccggacttg ggaccatcac agacttcaga accatcagga | 60 |
| acctgggagc aactgaaagc tgaactacag tgggctttca gacacacagc aggctgcgga | 120 |
| gcacaaatag gactggttcc ctccaggcca ccagcagggc ggtggaggtc ttcactgact | 180 |
| ccctgcctac ctctcaggac aatgtccttt tggctccaca gtccctgaag ccagagctgg | 240 |
| tgggggcagg gaggcagcca ccagcctcta tatgtagtgg aggagggggt gtccagggag | 360 |
| ggctgcatga tcctgagagc ccccacctca cccggctgga ctatcctccc acttcagggt | 360 |
| ttctctgggc ttccatcttg cccctgctga gccctgcttc ctcctctacc agcagcacaa | 420 |
| cccccaggct gggctcagag acctcatgtg gtgggatcac tcagtacccc gaggcggagg | 480 |
| gaaggaggga gggctgcagg gttccccttg gcctgcaaac aggaacacag ggtgtttctc | 540 |
| agtggctgcg agaatgctga tgaaaacccc aggatgttgt gtcaccgtgg tggccagctg | 600 |
| atagtgccaa tcatcccact ttgccctgag cactcctgca ggggtagaag actccagaac | 660 |
| cttctctcag gcccatggcc caagcagccc atg gaa ctt cat aac ctg agc tct | 714 |
| cca tct ccc tct ctc tcc tcc tct gtt ctc cct ccc tcc ttc tct ccc | 762 |
| tca ccc tcc tct gct ccc tct gcc ttt acc act gtg ggg ggg tcc tct | 810 |
| gga ggg ccc tgc cac ccc acc tct tcc tcg ctg gtg tct gcc ttc ctg | 858 |
| gca cca atc ctg gcc ctg gag ttt gtc ctg ggc ctg gtg ggg aac agt | 906 |
| ttg gcc ctc ttc atc ttc tgc atc cac acg cgg ccc tgg acc tcc aac | 954 |
| acg gtg ttc ctg gtc agc ctg gtg gcc gct gac ttc ctc ctg atc agc | 1002 |
| aac ctg ccc ctc cgc gtg gac tac tac ctc ctc cat gag acc tgg cgc | 1050 |
| ttt ggg gct gct gcc tgc aaa gtc aac ctc ttc atg ctg tcc acc aac | 1098 |
| cgc acg gcc agc gtt gtc ttc ctc aca gcc atc gca ctc aac cgc tac | 1146 |
| ctg aag gtg gtg cag ccc cac cac gtg ctg agc cgt gct tcc gtg ggg | 1194 |
| gca gct gcc cgg gtg gcc ggg gga ctc tgg gtg ggc atc ctg ctc ctc | 1242 |
| aac ggg cac ctg ctc ctg agc acc ttc tcc ggc ccc tcc tgc ctc agc | 1290 |
| tac agg gtg ggc acg aag ccc tcg gcc tcg ctc cgc tgg cac cag gca | 1338 |
| ctg tac ctg ctg gag ttc ttc ctg cca ctg gcg ctc atc ctc ttt gct | 1386 |
| att gtg agc att ggg ctc acc atc cgg aac cgt ggt ctg ggc ggg cag | 1434 |
| gca ggc ccg cag agg gcc atg cgt gtg ctg gcc atg gtg gtg gcc gtc | 1482 |
| tac acc atc tgc ttc ttg ccc agc atc atc ttt ggc atg gct tcc atg | 1530 |
| gtg gct ttc tgg ctg tcc gcc tgc cga tcc ctg gac ctc tgc aca cag | 1578 |
| ctc ttc cat ggc tcc ctg gcc ttc acc tac ctc aac agt gtc ctg gac | 1626 |
| ccc gtg ctc tac tgc ttc tct agc ccc aac ttc ctc cac cag agc cgg | 1674 |
| gcc ttg ctg ggc ctc acg cgg ggc cgg cag ggc cca gtg agc gac gag | 1722 |
| agc tcc tac caa ccc tcc agg cag tgg cgc tac cgg gag gcc tct agg | 1770 |
| aag gcg gag gcc ata ggg aag ctg aaa gtg cag ggc gag gtc tct ctg | 1818 |
| gaa aag gaa ggc tcc tcc cag ggc tga gggccagctg cagggctgca 1865 | |
| gcgctgtggg ggtaagggct gccgcgctct ggcctggagg gacaaggcca gcacacggtg | 1925 |
| cctcaaccaa ctggacaagg gatggcggca gaccaggggc caggccaaag cactggcagg | 1985 |
| actcatgtgg gtggcaggga gagaaaccca cctaggcctc tcagtgtgtc caggatggca | 2045 |
| ttcccagaat gcaggggaga gcaggatgcc gggtggagga gacaggcaag gtgccgccgg | 2105 |
| cacaccagct cagacagggg cctgcgcagc tgcaggggac agacgccaat cactgtcaca | 2165 |
| gcagagtcac cttagaaatt ggacagctgc atgttctgtg ctctccagtt tgtcccttcc | 2225 |
| aatattaata aacttccctt ttaaatatat ttatttgcag accaatatct gtctttaatt | 2285 |
| ctaacctggg actgtcagta ggcgtcaaag tgagcgcccc agtgaaggaa ccttggagag | 2345 |
| agtgggagca ttcccagcct tccaggggga ctcgtcttcc agactttgga gcccgcatgt | 2405 |
| ctgaagcaga ctctttcttg gtag 2429 | |

Also preferred is a polynucleotide comprising nucleotides 691-1842 of SEQ ID NO: 5, which represent the portion of SEQ ID NO: 5 that encodes CON103 amino acids. Nucleotides 1843-1845 represent a stop codon.

Another highly preferred polynucleotide of the invention comprises the sequence set forth in SEQ ID NO: 7, which comprises a CON203-encoding DNA sequence:

| | |
|---|---|
| ttgaatttag gtgacactat agaagagcta tgacgtcgca tgcacgcgta cgtaagctcg | 60 |
| gaattcggct cgagctgaac taatgactgc cgccataaga agacagagag aactgagtat | 120 |
| cctcccaaag gtgacactgg aagcaatgaa caccacagtg atgcaaggct tcaacagatc | 180 |
| tgagcggtgc cccagagaca ctcggatagt acagctggta ttcccagccc tctacacagt | 240 |
| ggttttcttg accggcatcc tgctgaatac tttggctctg tgggtgtttg ttcacatccc | 300 |
| cagctcctcc accttcatca tctacctcaa aaacactttg gtggccgact tgataatgac | 360 |
| actcatgctt cctttcaaaa tcctctctga ctcacacctg gcaccctggc agctcagagc | 420 |
| ttttgtgtgt cgtttttctt cggtgatatt ttatgagacc atgtatgtgg gcatcgtgct | 480 |
| gttagggctc atagcctttg acagattcct caagatcatc agacctttga gaaatatttt | 540 |
| tctaaaaaaa cctgtttttg caaaaacggt ctcaatcttc atctgggtct ttttggtctt | 600 |
| catctccctg ccaaatatga tcttgagcaa caaggaagca acaccatcgt ctgtgaaaaa | 660 |
| gtgtgcttcc ttaaaggggc ctctggggct gaaatggcat caaatggtaa ataacatatg | 720 |
| ccagtttatt ttctggactg gttttatcct aatgcttgtg ttttatgtgg ttattgcaaa | 780 |
| aaaagtatat gattcttata gaaagtccaa aagtaaggac agaaaaaaca acaaaaagct | 840 |
| ggaaggcaaa gtatttgttg tcgtggctgt cttctttgtg tgttttgctc catttcattt | 900 |
| tgccagagtt ccatatactc acagtcaaac caacaataag actgactgta gactgcaaaa | 960 |
| tcaactgttt attgctaaag aaacaactct ctttttggca gcaactaaca tttgtatgga | 1020 |
| tcccttaata tacatattct tatgtaaaaa attcacagaa aagctaccat gtatgcaagg | 1080 |
| gagaaagacc acagcatcaa gccaagaaaa tcatagcagt cagacagaca acataacctt | 1140 |
| aggctgacaa ctgtacatag ggttaacttc tatttattga tgagacttcc gtagataatg | 1200 |
| tggaaatcaa atttaaccaa gaaaaaaaga ttggaacaaa tgctctctta cattttattt | 1260 |
| atcctggtgt ccaggaaaag attatattaa atttaaatcc acatagatct attcataagc | 1320 |
| tgaatgaacc attacctaag agaatgcaac aggataccaa tggccactag aggcatattc | 1380 |
| cttcttcttt tttttttgtt aaatttcaag agcattcact ttacatttgg aaagactaag | 1440 |
| gggaacggtt atcctacaaa cctcccttca acacctttta catt 1484 | |

Also preferred is a polynucleotide comprising nucleotides 146-1144 of SEQ ID NO: 7, which represent the portion of SEQ ID NO: 7 that encodes CON203 amino acids. Nucleotides 1145-1147 represent a stop codon.

Another highly preferred polynucleotide of the invention comprises the sequence set forth in SEQ ID NO: 9, which comprises a human CON198 encoding DNA sequence:

| | |
|---|---|
| ATGATGGTGG ATCCCAATGG CAATGAATCC AGTGCTACAT ACTTCATCCT AATAGGCCTC | 60 |
| CCTGGTTTAG AAGAGGCTCA GTTCTGGTTG GCCTTCCCAT TGTGCTCCCT CTACCTTATT | 120 |
| GCTGTGCTAG GTAACTTGAC AATCATCTAC ATTGTGCGGA CTGAGCACAG CCTGCATGAG | 180 |
| CCCATGTATA TATTTCTTTG CATGCTTTCA GGCATTGACA TCCTCATCTC CACCTCATCC | 240 |
| ATGCCCAAAA TGCTGGCCAT CTTCTGGTTC AATTCCACTA CCATCCAGTT TGATGCTTGT | 300 |
| CTGCTACAGA TGTTTGCCAT CCACTCCTTA TCTGGCATGG AATCCACAGT GCTGCTGGCC | 360 |
| ATGGCTTTTG ACCGCTATGT GGCCATCTGT CACCCACTGC GCCATGCCAC AGTACTTACG | 420 |
| TTGCCTCGTG TCACCAAAAT TGGTGTGGCT GCTGTGGTGC GGGGGGCTGC ACTGATGGCA | 480 |
| CCCCTTCCTG TCTTCATCAA GCAGCTGCCC TTCTGCCGCT CCAATATCCT TTCCCATTCC | 540 |
| TACTGCCTAC ACCAAGATGT CATGAAGCTG GCCTGTGATG ATATCCGGGT CAATGTCGTC | 600 |
| TATGGCCTTA TCGTCATCAT CTCCGCCATT GGCCTGGACT CACTTCTCAT CTCCTTCTCA | 660 |
| TATCTGCTTA TTCTTAAGAC TGTGTTGGGC TTGACACGTG AAGCCCAGGC CAAGGCATTT | 720 |
| GGCACTTGCG TCTCTCATGT GTGTGCTGTG TTCATATTCT ATGTACCTTT CATTGGATTG | 780 |
| TCCATGGTGC ATCGCTTTAG CAAGCGGCGT GACTCTCCGC TGCCCGTCAT CTTGGCCAAT | 840 |
| ATCTATCTGC TGGTTCCTCC TGTGCTCAAC CCAATTGTCT ATGGAGTGAA GACAAAGGAG | 900 |
| ATTCGACAGC GCATCCTTCG ACTTTTCCAT GTGGCCACAC ACGCTTCAGA GCCCTAG | 957 |

The last three nucleotides of this sequence represent a stop codon.

Still another A highly preferred polynucleotide of the invention comprises the sequence set forth in SEQ ID NO: 11, which comprises a human CON197 encoding DNA sequence:

| | |
|---|---|
| ATGGAAAGCG AGAACAGAAG AGTGATAAGA GAATTCATCC TCCTTGGTCT GACCCAGTCT | 60 |
| CAAGATATTC AGCTCCTGGT CTTTGTGCTA GTTTTAATAT TCTACTTCAT CATCCTCCCT | 120 |
| GGAAATTTTC TCATTATTTT CACCATAAAG TCAGACCCTG GGCTCACAGC CCCCCTCTAT | 180 |
| TTCTTTCTGG GCAACTTGGC CTTCCTGGAT GCATCCTACT CCTTCATTGT GGCTCCCCGG | 240 |
| ATGTTGGTGG ACTTCCTCTC TGCGAAGAAG ATAATCTCCT ACAGAGGCTG CATCACTCAG | 300 |
| CTCTTTTTCT TGCACTTCCT TGGAGGAGGG GAGGGATTAC TCCTTGTTGT GATGGCCTTT | 360 |
| GACCGCTACA TCGCCATCTG CCGGCCTCTG CACTATCCTA CTGTCATGAA CCCTAGAACC | 420 |
| TGCTATGCAA TGATGTTGGC TCTGTGGCTT GGGGGTTTTG TCCACTCCAT TATCCAGGTG | 480 |
| GTCCTCATCC TCCGCTTGCC TTTTTGTGGC CCAAACCAGC TGGACAACTT CTTCTGTGAT | 540 |
| GTCCCACAGG TCATCAAGCT GGCCTGCACC GACACATTTG TGGTGGAGCT TCTGATGGTC | 600 |
| TTCAACAGTG GCCTGATGAC ACTCCTGTGC TTTCTGGGGC TTCTGGCCTC CTATGCAGTC | 660 |
| ATTCTTTGTC GCATACGAGG GTCTTCTTCT GAGGCAAAAA ACAAGGCCAT GTCCACGTGC | 720 |
| ATCACCCATA TCATTGTTAT ATTCTTCATG TTTGGACCTG GCATCTTCAT CTACACGCGC | 780 |
| CCCTTCAGGG CTTTCCCAGC TGACAAGGTG GTTTCTCTCT TCCACACAGT GATTTTTCCT | 840 |
| TTGTTGAATC CTGTCATTTA TACCCTTCGC AACCAGGAAG TGAAAGCTTC CATGAAAAAG | 900 |
| GTGTTTAATA AGCACATAGC CTGA 924 | |

The last three nucleotides of this sequence represent a stop codon.

Another highly preferred polynucleotide of the invention comprises the sequence set forth in SEQ ID NO: 13, which comprises a human CON202 encoding DNA sequence:

| | |
|---|---|
| 1 | TGCTTCCCCA TAAGGTAACA GCTTTGTTAG CNCTGTCTGA CATCATTGCT |
| 51 | TGTTWACTTA AGAACTGATA GGTYTTTTTT TTTTTTTTTT TTCAGATATT |
| 101 | CTGATGGCAA AACAAGTGGA AGAAAAGAGG AAGCATGACT GCAGATCAGA |
| 151 | TCAGTTCTCT TTGTGGATTA TATTTTCAGT AAAATGTATG GATCTATCTT |
| 201 | TTCCTTGTTC TTATATCTAG ATCATGAGAC TTGACTGAGG CTGTATCCTT |
| 251 | ATCCTCCATC CATCTATGGC GAACTATAGC CATGCAGCTG ACAACATTTT |
| 301 | GCAAAATCTC TCGCCTCTAA CAGCCTTTCT GAAACTGACT TCCTTGGGTT |
| 351 | TCATAATAGG AGTCAGCGTG GTGGGCAACC TCCTGATCTC CATTTTGCTA |
| 401 | GTGAAAGATA AGACCTTGCA TAGAGCACCT TACTACTTCC TGTTGGATCT |
| 451 | TTGCTGTTCA GATATCCTCA GATCTGCAAT TTGTTTCCCA TTTGTGTTCA |
| 501 | ACTCTGTCAA AAATGGTTCT ACCTGGACTT ATGGGACTCT GACTTGCAAA |
| 551 | GTGATTGCCT TTCTGGGGGT TTTGTCCTGT TTCCACACTG CTTTCATGCT |
| 601 | CTTCTGCATC AGTGTCACCA GATATTTAGC TATCGCCCAT CACCGCTTCT |
| 651 | ATACAAAGAG GCTGACCTTT TGGACGTGTC TGGCTGTGAT CTGTATGGTG |
| 701 | TGGACTCTGT CTGTGGCCAT GGCATTTCCC CCGGTTTTAG ACGTGGGCAC |
| 751 | TTACTCATTC ATTAGGGAGG AAGATCAATG CACCTTCCAA CACCGCTCCT |
| 801 | TCAGGGCTAA TGATTCCTTA GGATTTATGC TGCTTCTTGC TCTCATCCTC |
| 851 | CTAGCCACAC AGCTTGTCTA CCTCAAGCTG ATATTTTTCG TCCACGATCG |
| 901 | AAGAAAAATG AAGCCAGTCC AGTTTGTAGC AGCAGTCAGC CAGAACTGGA |
| 951 | CTTTTCATGG TCCTGGAGCC AGTGGCCAGG CAGCTGCCAA TTGGCTAGCA |
| 1001 | GGATTTGGAA GGGGTCCCAC ACCACCCACC TTGCTGGGCA TCAGGCAAAA |
| 1051 | TGCAAACACC ACAGGCAGAA GAAGGCTATT GGTCTTAGAC GAGTTCAAAA |
| 1101 | TGGAGAAAAG AATCAGCAGA ATGTTCTATA TAATGACTTT TCTGTTTCTA |
| 1151 | ACCTTGTGGG GCCCCTACCT GGTGGCCTGT TATTGGAGAG TTTTTGCAAG |
| 1201 | AGGGCCTGTA GTACCAGGGG GATTTCTAAC AGCTGCTGTC TGGATGAGTT |
| 1251 | TTGCCCAAGC AGGAATCAAT CCTTTTGTCT GCATTTTCTC AAACAGGGAG |
| 1301 | CTGAGGCGCT GTTTCAGCAC AACCCTTCTT TACTGCAGAA AATCCAGGTT |
| 1351 | ACCAAGGGAA CCTTACTGTG TTATATGAGG |

Also preferred is a polynucleotide comprising nucleotides 266-1375 of SEQ ID NO: 13, which represent the portion of SEQ ID NO: 13 that encodes CON202 amino acids. Nucleotides 1376-1378 represent a stop codon.

Another highly preferred polynucleotide of the invention comprises the sequence set forth in SEQ ID NO: 15, which comprises a human CON222 encoding DNA sequence:

| | |
|---|---|
| 1 | ATGTTTAGAC CTCTTGTGAA TCTCTCTCAC ATATATTTTA AGAAATTCCA |
| 51 | GTACTGTGGG TATGCACCAC ATGTTCGCAG CTGTAAACCA AACACTGATG |
| 101 | GAATTTCATC TCTAGAGAAT CTCTTGGCAA GCATTATTCA GAGAGTATTT |
| 151 | GTCTGGGTTG TATCTGCAGT TACCTGCTTT GGAAACATTT TTGTCATTTG |
| 201 | GATGCGACCT TATATCAGGT CTGAGAACAA GCTGTATGCC ATGTCAATCA |
| 251 | TTTCTCTCTG CTGTGCCGAC TGCTTAATGG GAATATATTT ATTCGTGATC |
| 301 | GGAGGCTTTG ACCTAAAGTT TCGTGGAGAA TACAATAAGC ATGCGCAGCT |
| 351 | GTGGATGGAG AGTACTCATT GTCAGCTTGT AGGATCTTTG GCCATTCTGT |
| 401 | CCACAGAAGT ATCAGTTTTA CTGTTAACAT TTCTGACATT GGAAAAATAC |
| 451 | ATCTGCATTG TCTATCCTTT TAGATGTGTG AGACCTGGAA AATGCAGAAC |
| 501 | AATTACAGTT CTGATTCTCA TTTGGATTAC TGGTTTTATA GTGGCTTTCA |
| 551 | TTCCATTGAG CAATAAGGAA TTTTTCAAAA ACTACTATGG CACCAATGGA |
| 601 | GTATGCTTCC CTCTTCATTC AGAAGATACA GAAAGTATTG GAGCCCAGAT |
| 651 | TTATTCAGTG GCAATTTTTC TTGGTATTAA TTTGGCCGCA TTTATCATCA |
| 701 | TAGTTTTTTC CTATGGAAGC ATGTTTTATA GTGTTCATCA AAGTGCCATA |
| 751 | ACAGCAACTG AAATACGGAA TCAAGTTAAA AAAGAGATGA TCCTTGCCAA |
| 801 | ACGTTTTTTC TTTATAGTAT TTACTGATGC ATTATGCTGG ATACCCATTT |
| 851 | TTGTAGTGAA ATTTCTTTCA CTGCTTCAGG TAGAAATACC AGGTACCATA |
| 901 | ACCTCTTGGG TAGTGATTTT TATTCTGCCC ATTAACAGTG CTTTGAACCC |
| 951 | AATTCTCTAT ACTCTGACCA CAAGACCATT TAAAGAAATG ATTCATCGGT |
| 1001 | TTTGGTATAA CTACAGACAA AGAAAATCTA TGGACAGCAA AGGTCAGAAA |
| 1051 | ACATATGCTC CATCATTCAT CTGGGTGGAA ATGTGGCCAC TGCAGGAGAT |
| 1101 | GCCACCTGAG TTAATGAAGC CGGACCTTTT CACATACCCC TGTGAAATGT |
| 1151 | CACTGATTTC TCAATCAACG AGACTCAATT CCTATTCA |

The last three nucleotides of this sequence represent a stop codon.

Another highly preferred polynucleotide of the invention comprises the sequence set forth in SEQ ID NO: 17, which comprises a human CON215 encoding DNA sequence. Also preferred is a polynucleotide comprising the portion of SEQ ID NO: 17 set forth below, which represent the portion of SEQ ID NO: 17 that encodes CON215 amino acids (the last three nucleotides represent a stop codon).

| | |
|---|---|
| ATGGGGTTCA ACTTGACGCT TGCAAAATTA CCAAATAACG AGCTGCACGG CCAAGAGAGT | 60 |
| CACAATTCAG GCAACAGGAG CGACGGGCCA GGAAAGAACA CCACCCTTCA CAATGAATTT | 120 |
| GACACAATTG TCTTGCCAGT GCTTTATCTC ATTATATTTG TGGCAAGCAT CTTGCTGAAT | 180 |
| GGTTTAGCAG TGTGGATCTT CTTCCACATT AGGAATAAAA CCAGCTTCAT ATTCTATCTC | 240 |
| AAAAACATAG TGGTTGCAGA CCTCATAATG ACGCTGACAT TTCCATTTCG AATAGTCCAT | 300 |
| GATGCAGGAT TTGGACCTTG GTACTTCAAG TTTATTCTCT GCAGATACAC TTCAGTTTTG | 360 |
| TTTTATGCAA ACATGTATAC TTCCATCGTG TTCCTTGGGC TGATAAGCAT TGATCGCTAT | 420 |
| CTGAAGGTGG TCAAGCCATT TGGGGACTCT CGGATGTACA GCATAACCTT CACGAAGGTT | 480 |
| TTATCTGTTT GTGTTTGGGT GATCATGGCT GTTTTGTCTT TGCCAAACAT CATCCTGACA | 540 |
| AATGGTCAGC CAACAGAGGA CAATATCCAT GACTGCTCAA AACTTAAAAG TCCTTTGGGG | 600 |
| GTCAAATGGC ATACGGCAGT CACCTATGTG AACAGCTGCT TGTTTGTGGC CGTGCTGGTG | 660 |
| ATTCTGATCG GATGTTACAT AGCCATATCC AGGTACATCC ACAAATCCAG CAGGCAATTC | 720 |
| ATAAGTCAGT CAAGCCGAAA GCGAAAACAT AACCAGAGCA TCAGGGTTGT TGTGGCTGTG | 780 |
| TTTTTTACCT GCTTTCTACC ATATCACTTG TGCAGAATTC CTTTTACTTT TAGTCACTTA | 840 |
| GACAGGCTTT TAGATGAATC TGCACAAAAA ATCCTATATT ACTGCAAAGA AATTACACTT | 900 |
| TTCTTGTCTG CGTGTAATGT TTGCCTGGAT CCAATAATTT ACTTTTTCAT GTGTAGGTCA | 960 |
| TTTTCAAGAA GGCTGTTCAA AAAATCAAAT ATCAGAACCA GGAGTGAAAG CATCAGATCA | 1020 |
| CTGCAAAGTG TGAGAAGATC GGAAGTTCTC ATATATTATG ATTATACTGA TGTGTAG | 1077 |

Another preferred polynucleotide of the invention comprises the portion of the sequence set forth in SEQ ID NO: 19 which comprises a human CON217 encoding DNA sequence:

| | |
|---|---|
| 1 | ATGTTAGCCA ACAGCTCCTC AACCAACAGT TCTGTTCTCC CGTGTCCTGA CTACCGACCT |
| 61 | ACCCACCGCC TGCACTTGGT GGTCTACAGC TTGGTGCTGG CTGCCGGGCT CCCCCTCAAC |
| 121 | GCGCTAGCCC TCTGGGTCTT CCTGCGCGCG CTGCGCGTGC ACTCGGTGGT GAGCGTGTAC |
| 181 | ATGTGTAACC TGGCGGCCAG CGACCTGCTC TTCACCCTCT CGCTGCCCGT TCGTCTCTCC |
| 241 | TACTACGCAC TGCACCACTG GCCCTTCCCC GACCTCCTGT GCCAGACGAC GGGCGCCATC |
| 301 | TTCCAGATGA ACATGTACGG CAGCTGCATC TTCCTGATGC TCATCAACGT GGACCGCTAC |
| 361 | GCCGCCATCG TGCACCCGCT GCGACTGCGC CACCTGCGGC GGCCCCGCGT GGCGCGGCTG |
| 421 | CTCTGCCTGG GCGTGTGGGC GCTCATCCTG GTGTTTGCCG TGCCCGCCGC CCGCGTGCAC |
| 481 | AGGCCCTCGC GTTGCCGCTA CCGGGACCTC GAGGTGCGCC TATGCTTCGA GAGCTTCAGC |
| 541 | GACGAGCTGT GGAAAGGCAG GCTGCTGCCC CTCGTGCTGC TGGCCGAGGC GCTGGGCTTC |
| 601 | CTGCTGCCCC TGGCGGCGGT GGTCTACTCG TCGGGCCGAG TCTTCTGGAC GCTGGCGCGC |
| 661 | CCCGACGCCA CGCAGAGCCA GCGGCGGCGG AAGACCGTGC GCCTCCTGCT GGCTAACCTC |
| 721 | GTCATCTTCC TGCTGTGCTT CGTGCCCTAC AACAGCACGC TGGCGGTCTA CGGGCTGCTG |
| 781 | CGGAGCAAGC TGGTGGCGGC CAGCGTGCCT GCCCGCGATC GCGTGCGCGG GGTGCTGATG |
| 841 | GTGATGGTGC TGCTGGCCGG CGCCAACTGC GTGCTGGACC CGCTGGTGTA CTACTTTAGC |
| 901 | GCCGAGGGCT TCCGCAACAC CCTGCGCGGC CTGGGCACTC CGCACCGGGC CAGGACCTCG |
| 961 | GCCACCAACG GGACGCGGGC GGCGCTCGCG CAATCCGAAA GGTCCGCCGT CACCACCGAC |
| 1021 | GCCACCAGGC CGGATGCCGC CAGTCAGGGG CTGCTCCGAC CCTCCGACTC CCACTCTCTG |
| 1081 | TCTTCCTTCA CACAGTGTCC CCAGGATTCC GCCCTCTGA |

The last three nucleotides of this sequence represent a stop codon.

The invention also includes polynucleotides differing, from the sequences set forth in SEQ ID NOS: 1, 3, 5, 7, 9, 11, 13, 15, 17 and 19 and from their complementary strand by at least one nucleotide.

In a related embodiment, the invention provides vectors comprising a polynucleotide of the invention. Such vectors are useful, e.g., for amplifying the polynucleotides in host cells to create useful quantities thereof. In preferred embodiments, the vector is an expression vector wherein the polynucleotide of the invention is operatively linked to a polynucleotide comprising an expression control sequence. Such vectors are useful for recombinant production of polypeptides of the invention.

In another related embodiment, the invention provides host cells that are transformed or transfected (stably or transiently) with a polynucleotide of the invention or vectors of the invention. As stated above, such host cells are useful for amplifying the polynucleotides and also for expressing the GPCR seven transmembrane receptor polypeptides or fragments thereof encoded by the polynucleotides. Such host cells are useful in assays as described herein.

In still another related embodiment, the invention provides a method for producing a seven transmembrane receptor polypeptide (or fragment thereof) of the invention comprising the steps of growing a host cell of the invention in a nutrient medium and isolating the polypeptide or variant thereof from the cell or the medium. Since the GPCR polypeptides are seven transmembrane receptors, it will be appreciated that, for some applications, such as certain activity assays, the preferable isolation may involve isolation of cell membranes containing the polypeptide embedded therein, whereas for other applications a more complete isolation may be preferable.

In still another embodiment, the invention provides antibodies that are specific for the GPCR seven transmembrane receptors of the invention. Antibody specificity is described in greater detail below. However, it should be emphasized that antibodies that can be generated from polypeptides that have previously been described in the literature and that are capable of fortuitously cross-reacting with the GPCR polypeptides of the invention (e.g., due to the fortuitous existence of a similar epitope in both polypeptides) are considered “cross-reactive” antibodies. Such cross-reactive antibodies are not antibodies that are “specific” for the GPCR polypeptides. The determination of whether an antibody is specific for a GPCR polypeptide or is cross-reactive with another known receptor is made using Western blotting assays or several other assays well known in the literature. For identifying cells that express GPCR polypeptides and also for modulating GPCR-ligand binding activity, antibodies that specifically bind to an extracellular epitope of one of the GPCR seven transmembrane receptors of the present invention are preferred.

In one preferred variation, the invention provides monoclonal antibodies. Hybridomas that produce such antibodies also are intended as aspects of the invention. In yet another variation, the invention provides a humanized antibody. Humanized antibodies are useful for in vivo therapeutic indications.

In another variation, the invention provides a cell-free composition comprising polyclonal antibodies, wherein at least one of the antibodies is an antibody of the invention specific for a GPCR polypeptide of the present invention. Antisera isolated from an animal is an exemplary composition, as is a composition comprising an antibody fraction of an antisera that has been resuspended in water or in another diluent, excipient, or carrier.

In still another related embodiment, the invention provides anti-idiotypic antibodies specific for an antibody that is specific for a GPCR polypeptide of the present invention.

It is well known that antibodies contain relatively small antigen binding domains that can be isolated chemically or by recombinant techniques. Such domains are useful GPCR binding molecules themselves, and also may be reintroduced into human antibodies, or fused to toxins or other polypeptides. Thus, in still another embodiment, the invention provides a polypeptide comprising a fragment of a GPCR-specific antibody, wherein the fragment and the polypeptide bind to a GPCR seven transmembrane receptor of the present invention. By way of non-limiting example, the invention provides polypeptides that are single chain antibodies and CDR-grafted antibodies.

Also within the scope of the invention are compositions comprising polypeptides, polynucleotides, or antibodies of the invention that have been formulated with, e.g., a pharmaceutically acceptable carrier.

The invention also provides methods of using antibodies of the invention. For example, the invention provides a method for modulating ligand binding of a GPCR seven transmembrane receptor of the present invention comprising the step of contacting the seven transmembrane receptor with an antibody specific for the seven transmembrane receptor, under conditions wherein the antibody binds the receptor.

GPCR polypeptides are expressed in the brain, providing an indication that aberrant GPCR polypeptide signaling activity may correlate with one or more neurological disorders. The invention also provides a method for treating a neurological disorder comprising the step of administering to a mammal in need of such treatment an amount of an antibody-like polypeptide of the invention that is sufficient to modulate ligand binding of a GPCR seven transmembrane receptor of the present invention in neurons of the mammal. In addition to administration of antibody-like polypeptides, administration of natural ligands for GPCR polypeptides as well as modulators of GPCR polypeptide activity, such as small molecules that mimic, agonize or antagonize ligand-mediated GPCR polypeptide signaling, are contemplated. The expression pattern provides an indication that such molecules will have utility for treating neurological and/or psychiatric diseases, including but hot limited to schizophrenia, depression, anxiety, bipolar disease, affective disorders, attention deficit hyperactivity disorder/attention deficit disorder (ADHD/ADO), epilepsy, neuritis, neurasthenia, neuropathy, neuroses, Alzheimer's disease, Parkinson's disease, migraine, senile dementia, and the like. Treatment of individuals having any of these disorders is contemplated as an aspect of the invention.

Thus, in yet another embodiment, the invention provides genetic screening procedures that entail analyzing a person's genome—in particular their alleles for GPCR's of the invention—to determine whether the individual possesses a genetic characteristic found in other individuals that are considered to be afflicted with, or at risk for, developing a mental disorder or disease of the brain that is suspected of having a hereditary component. For example, in one embodiment, the invention provides a method for determining a potential for developing a disorder affecting the brain in a human subject comprising the steps of analyzing the coding sequence of one or more GPCR genes from the human subject; and determining development potential for the disorder in said human subject from the analyzing step.

More particularly, the invention provides a method of screening a human subject to diagnose a disorder affecting the brain or genetic predisposition therefor, comprising the steps of: (a) assaying nucleic acid of a human subject to determine a presence or an absence of a mutation altering the amino acid sequence, expression, or biological activity of at least one seven transmembrane receptor that is expressed in the brain, wherein the seven transmembrane receptor comprises an amino acid sequence selected from the group consisting of SEQ ID NOs: 2, 4, 6, 8, 10, 12, 14, 16, 18, and 20, or an allelic variant thereof, and wherein the nucleic acid corresponds to the gene encoding the seven transmembrane receptor; and (b) diagnosing the disorder or predisposition from the presence or absence of said mutation, wherein the presence of a mutation altering the amino acid sequence, expression, or biological activity of allele in the nucleic acid correlates with an increased risk of developing the disorder. In preferred variations, the seven transmembrane receptor is CON202 comprising an amino acid sequence set forth in SEQ ID NO: 14, or an allelic variant thereof, and the disease is schizophrenia.

By “human subject” is meant any human being, human embryo, or human fetus. It will be apparent that methods of the present invention will be of particular interest to individuals that have themselves been diagnosed with a disorder affecting the brain or have relatives that have been diagnosed with a disorder affecting the brain.

By “screening for an increased risk” is meant determination of whether a genetic variation exists in the human subject that correlates with a greater likelihood of developing a disorder affecting the brain than exists for the human population as a whole. or for a relevant racial or ethnic human sub-population to which the individual belongs. Both positive and negative determinations (i.e., determinations that a genetic predisposition marker is present or is absent) are intended to fall within the scope of screening methods of the invention. In preferred embodiments, the presence of a mutation altering the sequence or expression of at least one CON202 seven transmembrane receptor allele in the nucleic acid is correlated with an increased risk of developing schizophrenia, whereas the absence of such a mutation is reported as a negative determination.

The “assaying” step of the invention may involve any techniques available for analyzing nucleic acid to determine its characteristics, including but not limited to well-known techniques such as single-strand conformation polymorphism analysis (SSCP) [Orita et al., Proc Natl. Acad. Sci. USA, 86: 2766-2770 (1989)]; heteroduplex analysis [White et al., Genomics, 12: 301-306 (1992)]; denaturing gradient gel electrophoresis analysis [Fischer et al., Proc. Natl. Acad. Sci. USA, 80: 1579-1583 (1983); and Riesner et al., Electrophoresis, 10: 377-389 (1989)]; DNA sequencing; RNase cleavage [Myers et al., Science, 230: 1242-1246 (1985)]; chemical cleavage of mismatch techniques [Rowley et al., Genomics, 30: 574-582 (1995); and Roberts et al., Nucl. Acids Res., 25: 3377-3378 (1997)]; restriction fragment length polymorphism analysis; single nucleotide primer extension analysis [Shumaker et al., Hum. Mutat., 7: 346-354 (1996); and Pastinen et al., Genome Res., 7: 606-614 (1997)]; 5′ nuclease assays [Pease et al., Proc. Natl. Acad. Sci. USA, 91:5022-5026 (1994)]; DNA Microchip analysis [Ramsay, G., Nature Biotechnology, 16: 40-48 (1999); and Chee et al., U.S. Pat. No. 5,837,832]; and ligase chain reaction [Whiteley et al., U.S. Pat. No. 5,521,065]. [See generally, Schafer and Hawkins, Nature Biotechnology, 16: 33-39 (1998).] All of the foregoing documents are hereby incorporated by reference in their entirety.

Thus, in one preferred embodiment involving screening CON202 sequences, for example, the assaying step comprises at least one procedure selected from the group consisting of: (a) determining a nucleotide sequence of at least one codon of at least one CON202 allele of the human subject; (b) performing a hybridization assay to determine whether nucleic acid from the human subject has a nucleotide sequence identical to or different from one or more reference sequences; (c) performing a polynucleotide migration assay to determine whether nucleic acid from the human subject has a nucleotide sequence identical to or different from one or more reference sequences; and (d) performing a restriction endonuclease digestion to determine whether nucleic acid from the human subject has a nucleotide sequence identical to or different from one or more reference sequences.

In a highly preferred embodiment, the assaying involves sequencing of nucleic acid to determine nucleotide sequence thereof, using any available sequencing technique. [See, e.g., Sanger et al., Proc. Natl. Acad. Sci. (USA), 74: 5463-5467 (1977) (dideoxy chain termination method); Mirzabekov, TIBTECH, 12: 27-32 (1994) (sequencing by hybridization); Drmanac et al., Nature Biotechnology, 16: 54-58 (1998); U.S. Pat. No. 5,202,23 1; and Science, 260: 1649-1652 (1993) (sequencing by hybridization); Kieleczawa et al., Science, 258: 1787-1791 (1992) (sequencing by primer walking); (Douglas et al., Biotechniques, 14: 824-828 (1993) (Direct sequencing of PCR products); and Akane et al., Biotechniques 16: 238-241 (1994); Maxam and Gilbert, Meth. Enzymol., 65: 499-560 (1977) (chemical termination sequencing), all incorporated herein by reference.] The analysis may entail sequencing of the entire seven transmembrane receptor gene genomic DNA sequence, or portions thereof; or sequencing of the entire seven transmembrane receptor coding sequence or portions thereof. In some circumstances, the analysis may involve a determination of whether an individual possesses a particular allelic variant, in which case sequencing of only a small portion of nucleic acid—enough to determine the sequence of a particular codon characterizing the allelic variant—is sufficient. This approach is appropriate, for example, when assaying to determine whether one family member inherited the same allelic variant that has been previously characterized for another family member, or, more generally, whether a person's genome contains an allelic variant that has been previously characterized and correlated with a mental disorder having a heritable component.

In another highly preferred embodiment, the assaying step comprises performing a hybridization assay to determine whether nucleic acid from the human subject has a nucleotide sequence identical to or different from one or more reference sequences. In a preferred embodiment, the hybridization involves a determination of whether nucleic acid derived from the human subject will hybridize with one or more oligonucleotides. wherein the oligonucleotides have nucleotide sequences that correspond identically to a portion of the GPCR gene sequence taught herein, such as the CON202 coding sequence set forth in SEQ ID NO: 14, or that correspond identically except for one mismatch. The hybridization conditions are selected to differentiate between perfect sequence complementarity and imperfect matches differing by one or more bases. Such hybridization experiments thereby can provide single nucleotide polymorphism sequence information about the nucleic acid from the human subject, by virtue of knowing the sequences of the oligonucleotides used in the experiments.

Several of the techniques outlined above involve an analysis wherein one performs a polynucleotide migration assay, e.g., on a polyacrylamide electrophoresis gel (or in a capillary electrophoresis system), under denaturing or non-denaturing conditions. Nucleic acid derived from the human subject is subjected to gel electrophoresis, usually adjacent to (or co-loaded with) one or more reference nucleic acids, such as reference GPCR-encoding sequences having a coding sequence identical to all or a portion of SEQ ID NO: 1, 3, 5, 7, 9, 11, 13, 15, 17, or 19 (or identical except for one known polymorphism). The nucleic acid from the human subject and the reference sequence(s) are subjected to similar chemical or enzymatic treatments and then electrophoresed under conditions whereby the polynucleotides will show a differential migration pattern, unless they contain identical sequences. [See generally Ausubel et al. (eds.), Current Protocols in Molecular Biology, New York: John Wiley & Sons, Inc. (1987-1999); and Sambrook et al., (eds.), Molecular Cloning, A Laboratory Manual, Cold Spring Harbor, New York: Cold Spring Harbor Laboratory Press (1989), both incorporated herein by reference in their entirety.]

In the context of assaying, the term “nucleic acid of a human subject” is intended to include nucleic acid obtained directly from the human subject (e.g., DNA or RNA obtained from a biological sample such as a blood, tissue, or other cell or fluid sample); and also nucleic acid derived from nucleic acid obtained directly from the human subject. By way of non-limiting examples, well known procedures exist for creating cDNA that is complementary to RNA derived from a biological sample from a human subject, and for amplifying (e.g., via polymerase chain reaction (PCR)) DNA or RNA derived from a biological sample obtained from a human subject. Any such derived polynucleotide which retains relevant nucleotide sequence information of the human subject's own DNA/RNA is intended to fall within the definition of “nucleic acid of a human subject” for the purposes of the present invention.

In the context of assaying, the term “mutation” includes addition, deletion, and/or substitution of one or more nucleotides in the GPCR gene sequence e.g., as compared to the seven transmembrane receptor-encoding sequences set forth in SEQ D NO: 1, 3, 5, 7, 9, 11, 13, 15, 17, or 19) and other polymorphisms that occur in introns (where introns exist) and that are identifiable via sequencing, restriction fragment length polymorphism, or other techniques. The various activity examples provided herein permit determination of whether a mutation modulates activity of the relevant receptor in the presence or absence of various test substances.

In a related embodiment, the invention provides methods of screening a person's genotype with respect to GPCR's of the invention, and correlating such genotypes with diagnoses for disease or with predisposition for disease (for genetic counseling). For example, the invention provides a method of screening for a CON202 hereditary schizophrenia genotype in a human patient, comprising the steps of: (a) providing a biological sample comprising nucleic acid from the patient, the nucleic acid including sequences corresponding to said patient's CON202 alleles; (b) analyzing the nucleic acid for the presence of a mutation or mutations; (c) determining a CON202 genotype from the analyzing step; and (d) correlating the presence of a mutation in a CON202 allele with a hereditary schizophrenia genotype. In a preferred embodiment, the biological sample is a cell sample containing human cells that contain genomic DNA of the human subject. The analyzing can be performed analogously to the assaying described in preceding paragraphs. For example, the analyzing comprises sequencing a portion of the nucleic acid (e.g., DNA or RNA), the portion comprising at least one codon of the CON202 alleles.

Although more time consuming and expensive than methods involving nucleic acid analysis, the invention also may be practiced by assaying protein of a human subject to determine the presence or absence of an amino acid sequence variation in GPCR protein from the human subject. Such protein analyses may be performed, e.g., by fragmenting GPCR protein via chemical or enzymatic methods and sequencing the resultant peptides; or by Western analyses using an antibody having specificity for a particular allelic variant of the GPCR.

The invention also provides materials that are useful for performing methods of the invention. For example, the present invention provides oligonucleotides useful as probes in the many analyzing techniques described above. In general, such oligonucleotide probes comprise 6, 7, 8,9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides that have a sequence that is identical, or exactly complementary, to a portion of a human GPCR gene sequence taught herein (or allelic variant thereof), or that is identical or exactly complementary except for one nucleotide substitution. In a preferred embodiment, the oligonucleotides have a sequence that corresponds in the foregoing manner to a human GPCR coding sequence taught herein, and in particular, the coding sequences set forth in SEQ ID NOs: 1, 3, 5, 7, 9, 11, 13, 15, 17, or 19. In one variation, an oligonucleotide probe of the invention is purified and isolated. In another variation, the oligonucleotide probe is labeled, e.g., with a radioisotope, chromophore, or fluorophore. In yet another variation, the probe is covalently attached to a solid support. [See generally Ausubel et al. And Sambrook et al., supra.]

In a related embodiment, the invention provides kits comprising reagents that are useful for practicing methods of the invention. For example, the invention provides a kit for screening a human subject to diagnose schizophrenia or a genetic predisposition therefor, comprising, in association: (a) an oligonucleotide useful as a probe for identifying polymorphisms in a human CON202 seven transmembrane receptor gene, the oligonucleotide comprising 6-50 nucleotides that have a sequence that is identical or exactly complementary to a portion of a human CON202 gene sequence or CON202 coding sequence, except for one sequence difference selected from the group consisting of a nucleotide addition, a nucleotide deletion. or nucleotide substitution; and (b) a media packaged with the oligonucleotide containing information identifying polymorphisms identifyable with the probe that correlate with schizophrenia or a genetic predisposition therefor. Exemplary information-containing media include printed paper package inserts or packaging labels; and magnetic and optical storage media that are readable by computers or machines used by practitioners who perform genetic screening and counseling services. The practitioner uses the information provided in the media to correlate the results of the analysis with the oligonucleotide with a diagnosis. In a preferred variation, the oligonucleotide is labeled.

In still another embodiment, the invention provides methods of identifying those allelic variants of GPCR's of the invention that correlate with mental disorders. For example, the invention provides a method of identifying a seven transmembrane allelic variant that correlates with a mental disorder, comprising steps of: (a) providing a biological sample comprising nucleic acid from a human patient diagnosed with a mental disorder, or from the patient's genetic progenitors or progeny; (b) analyzing the nucleic acid for the presence of a mutation or mutations in at least one seven transmembrane receptor that is expressed in the brain, wherein the at least one seven transmembrane receptor comprises an amino acid sequence selected from the group consisting of SEQ ID NOs: 2, 4, 6, 8, 10, 12, 14, 16, 18, and 20, or an allelic variant thereof, and wherein the nucleic acid includes sequence corresponding to the gene or genes encoding the at least one seven transmembrane receptor; (c) determining a genotype for the patient for the at least one seven transmembrane receptor from said analyzing step; and (d) identifying an allelic variant that correlates with the mental disorder from the determining step. To expedite this process, it may be desirable to perform linkage studies in the patients (and possibly their families) to correlate chromosomal markers with disease states. The chromosomal localization data provided herein facilitates identifying an involved GPCR with a chromosomal marker.

The foregoing method can be performed to correlate GPCR's of the invention to a number of disorders having hereditary components that are causative or that predispose persons to the disorder. For example, in one preferred variation. the disorder is schizophrenia, and the at least one seven transmembrane receptor comprises CON202 having an amino acid sequence set forth in SEQ ID NO: 14, or an allelic variant thereof.

Also contemplated as part of the invention are polynucleotides that comprise the allelic variant sequences identified by such methods, and polypeptides encoded by the allelic variant sequences, and oligonucleotide and oligopeptide fragments therof that embody the mutations that have been identified. Such materials are useful in in vitro cell-free and cell-based assays for idenifying lead compounds and therapeutics for treatment of the disorders. For example, the variants are used in activity assays, binding assays, and assays to screen for activity modulators described herein. In one preferred embodiment, the invention provides a purified and isolated polynucleotide comprising a nucleotide sequence encoding a CON202 receptor allelic variant identified according to the methods described above; and an oligonucleotide that comprises the sequences that differentiate the allelic variant from the CON202 sequences set forth in SEQ ID NOs: 13 and 14. The invention also provides a vector comprising the polynucleotide (preferably an expression vector); and a host cell transformed or transfected with the polynucleotide or vector. The invention also provides an isolated cell line that is expressing the allelic variant GPCR polypeptide; purified cell membranes from such cells; purified polypeptide; and synthetic peptides that embody the allelic variation amino acid sequence. In one particular embodiment, the invention provides a purified polynucleotide comprising a nucleotide sequence encoding a CON202 seven transmembrane receptor protein of a human that is affected with schizophrenia; wherein said polynucleotide hybridizes to the complement of SEQ ID NO: 13 under the following hybridization conditions: (a) hybridization for 16 hours at 42° C. in a hybridization solution comprising 50% formamide, 1% SDS, 1 M NaCl, 10% dextran sulfate and (b) washing 2 times for 30 minutes at 60° C. in a wash solution comprising 0.1×SSC and 1% SDS; and wherein the polynucleotide encodes a CON202 amino acid sequence that differs from SEQ ID NO: 14 at at least one residue.

An examplary assay for using the allelic variants is a method for identifying a modulator of CON202 biological activity, comprising the steps of: (a) contacting a cell expressing the allelic variant in the presence and in the absence of a putative modulator compound: (b) measuring CON202 biological activity in the cell; and (c) identifying a putative modulator compound in view of decreased or increased CON202 biological activity in the presence versus absence of the putative modulator.

In still another example, the invention provides for a method of diagnosing schizophrenia or a susceptibility to schizophrenia comprising the steps of: determining the presence or amount of expression of CON202 polypeptide as set out as SEQ ID NO: 14 or the polypeptide encoded by the nucleic acid molecule having SEQ ID NO: 13 in a sample; and comparing the level of CON202 polypeptide in a biological, tissue or cellular sample from normal subjects or the subject at an earlier time, wherein the susceptibility to schizophrenia is based on the presence or amount of CON202 polypeptide expression.

The invention also provides for a method of treating schizophrenia comprising the step of administering to a human diagnosed with schizophrenia an amount of a modulator of CON202 receptor activity sufficient to modulate CON202 receptor activity or CON202 ligand binding in said human.

The invention also provides assays to identify compounds that bind GPCR seven transmembrane receptors. One such assay comprises the steps of: (a) contacting a composition comprising one of the GPCR seven transmembrane receptor polypeptides of the invention with a compound suspected of binding a GPCR polypeptide of the invention; and (b) measuring binding between the compound and the GPCR polypeptide. In one variation, the composition comprises a cell expressing a GPCR polypeptide of the invention on its surface. In another variation, an isolated GPCR polypeptide of the invention or cell membranes comprising a GPCR polypeptide of the invention are employed. The binding may be measured directly, e.g., using a labeled compound, or may be measured indirectly by several techniques, including measuring intracellular signaling of a GPCR polypeptide of the invention induced by the compound (or measuring changes in the level of GPCR polypeptide signaling).

The invention also provides a method for identifying a modulator of binding between a GPCR seven transmembrane receptor of the invention and a GPCR polypeptide binding partner, comprising the steps of: (a) contacting a GPCR polypeptide binding partner and a composition comprising one of the GPCR seven transmembrane receptors of the invention in the presence and in the absence of a putative modulator compound; (b) detecting binding between the binding partner and the GPCR polypeptide of the invention; and (c) identifying a putative modulator compound in view of decreased or increased binding between the binding partner and the GPCR polypeptide in the presence of the putative modulator, as compared to binding in the absence of the putative modulator.

GPCR polypeptide binding partners that stimulate GPCR seven transmembrane receptors of the present invention are useful as agonists in disease states characterized by insufficient GPCR polypeptide signaling (e.g., as a result of insufficient expression of active GPCR polypeptide ligand). GPCR polypeptide binding partners that block ligand-mediated GPCR polypeptide signaling are useful as GPCR polypeptide antagonists to treat disease states characterized by excessive GPCR polypeptide signaling.

Additional features and variations of the invention will be apparent to those skilled in the art from the entirety of this application, including the detailed description, and all such features are intended as aspects of the invention. Likewise, features of the invention described herein can be re-combined into additional embodiments that also are intended as aspects of the invention, irrespective of whether the combination of features is specifically mentioned above as an aspect or embodiment of the invention. Also, only such limitations which are described herein as critical to the invention should be viewed as such; variations of the invention lacking limitations which have not been described herein as critical are intended as aspects of the invention.

In addition to the foregoing, the invention includes, as an additional aspect, all embodiments of the invention narrower in scope in any way than the variations specifically mentioned above. Although the applicant(s) invented the full scope of the claims appended hereto, the claims appended hereto are not intended to encompass within their scope the prior art work of others. Therefore, in the event that statutory prior art within the scope of a claim is brought to the attention of the applicants by a Patent Office or other entity or individual, the applicant(s) reserve the right to exercise amendment rights under applicable patent laws to redefine the subject matter of such a claim to specifically exclude such statutory prior art or obvious variations of statutory prior art from the scope of such a claim. Variations of the invention defined by such amended claims also are intended as aspects of the invention.

### DETAILED DESCRIPTION OF THE INVENTION

The present invention provides purified and isolated polynucleotides (e.g., DNA sequences and RNA transcripts, both sense and complementary antisense strands, both single and double stranded, including splice variants thereof) encoding human G protein-coupled receptors referred to herein as GPCR polypeptides. DNA polynucleotides of the invention include genomic DNA, cDNA, and DNA that has been chemically synthesized in whole or in part. “Synthesized” as used herein and understood in the art, refers to polynucleotides produced by purely chemical, as opposed to enzymatic, methods. “Wholly” synthesized DNA sequences are therefore produced entirely by chemical means, and “partially” synthesized DNAs embrace those wherein only portions of the resulting DNA were produced by chemical means.

Genomic DNA of the invention comprises the protein coding region for a polypeptide of the invention and is also intended to include allelic variants thereof. It is widely understood that, for many genes, genomic DNA is transcribed into RNA transcripts that undergo one or more splicing events wherein intron (i.e., non-coding regions) of the transcripts are removed, or “spliced out.” RNA transcripts that can be spliced by alternative mechanisms, and therefore be subject to removal of different RNA sequences but still encode a GPCR polypeptide of the present invention, are referred to in the art as splice variants which are embraced by the invention. Splice variants comprehended by the invention therefore are encoded by the same original genomic DNA sequences but arise from distinct mRNA transcripts. Allelic variants are modified forms of a wild type gene sequence, the modification resulting from recombination during chromosomal segregation or exposure to conditions which give rise to genetic mutation. Allelic variants. like wild type genes, are naturally occurring sequences (as opposed to non-naturally occurring variants which arise from in vitro manipulation).

The invention also comprehends cDNA that is obtained through reverse transcription of an RNA polynucleotide encoding a GPCR of the present invention (conventionally followed by second strand synthesis of a complementary strand to provide a double-stranded DNA).

A preferred DNA sequence encoding a human GPCR polypeptide is set out in SEQ ID NO: 1, wherein nucleotides 157 to 1122 represent the CON193 coding sequence, with termination codon (surrounded by upstream and downstream untranslated sequences). Another preferred DNA sequence encoding a human GPCR polypeptide is set out in SEQ ID NO: 3, wherein nucleotides 1 to 1014 represent the CON166 coding sequence and stop codon. Still another preferred DNA sequence encoding a human GPCR polypeptide is set out in SEQ ID NO: 5, wherein nucleotides 691 to 1845 represent the CON103 coding sequence with stop codon (surrounded by upstream and downstream untranslated sequences). Another preferred DNA sequence encoding a human GPCR polypeptide is set out in SEQ ID NO: 7, wherein nucleotides 146 to 1147 represent the CON203 coding sequence with stop codon (surrounded by upstream and downstream untranslated sequences). A preferred DNA sequence encoding a human GPCR polypeptide is set out in SEQ ID NO: 9, wherein nucleotides 1 to 957 represent the CON198 coding sequence with stop codon. Another preferred DNA sequence encoding a human GPCR polypeptide is set out in SEQ ID NO: 11, wherein nucleotides 1 to 924 represent the CON197 coding sequence with stop codon (followed by downstream untranslated sequences). A preferred DNA sequence encoding a human GPCR polypeptide is set out in SEQ ID NO: 13, wherein nucleotides 266 to 1378 represent the CON202 coding sequence and termination codon (surrounded by upstream and downstream untranslated sequences). A preferred DNA sequence encoding a human GPCR polypeptide is set out in SEQ ID NO: 15, wherein nucleotides 1 to 1191 represent the CON222 coding sequence and termination codon. A preferred DNA sequence encoding a human GPCR polypeptide is set out in SEQ ID NO: 17, wherein nucleotides 13 to 1089 represent tile CON215 coding sequence and termination codon (surrounded by upstream and downstream untranslated sequences). A preferred DNA sequence encoding a human GPCR polypeptide is set out in SEQ ID NO: 19, wherein nucleotides 42 to 1157 represent the CON217 coding sequence (surrounded by upstream and downstream untranslated sequences). The foregoing sequences without their termination codons also comprise preferred sequences.

The worker of skill in the art will readily appreciate that the preferred DNA of the invention comprises a double stranded molecule, for example the molecule having any one of the sequences set forth in SEQ ID NOS: 1, 3, 5, 7, 9, 11, 13, 15, 17, or 19 (or coding portions thereof) along with the complementary molecule (the “non-coding strand” or “complement”) having a sequence deducible from the sequence of SEQ ID NOS: 1, 3, 5, 7, 9, 11, 13, 15, 17, or 19 according to Watson-Crick base pairing rules for DNA. Also preferred are other polynucleotides encoding the GPCR polypeptides of the invention set forth in SEQ ID NOS: 2, 4, 6, 8, 10, 12, 14, 16, 18 and 20 which differ in sequence from the polynucleotide of SEQ ID NOS: 1, 3, 5, 7, 9, 11, 13, 15, 17, or 19, respectively, by virtue of the well-known degeneracy of the universal genetic code.

The invention further embraces species, preferably mammalian, homologs of the human GPCR DNAs. Species homologs, sometimes referred to as “orthologs,” in general, share at least 35%, at least 40%, at least 45%, at least 50%, at least 60%, at least 65%, at least 70%, at least 75%, at least 80%, at least 85%, at least 90%, at least 95%, at least 98%, or at least 99% homology with human DNA of the invention. Percent sequence “homology” with respect to polynucleotides of the invention is defined herein as the percentage of nucleotide bases in the candidate sequence that are identical to nucleotides in the GPCR sequence set forth in any one of SEQ ID NOS: 1, 3, 5, 7, 9, 11, 13, 15, 17, or 19 after aligning the sequences and introducing gaps, if necessary, to achieve the maximum percent sequence identity.

The polynucleotide sequence information provided by the invention makes possible large scale expression of the encoded polypeptide by techniques well known and routinely practiced in the art. Polynucleotides of the invention also permit identification and isolation of polynucleotides encoding related GPCR polypeptides. such as human allelic variants and species homologs, by well known techniques including Southern and/or Northern hybridization, and polymerase chain reaction (PCR). Examples of related polynucleotides include human and non-human genomic sequences, including allelic variants. as well as polynucleotides encoding polypeptides homologous to GPCR polypeptides and structurally related the polypeptides sharing one or more biological, immunological, and/or physical properties of the GPCR polypeptides. Non-human species genes encoding proteins homologous to GPCR polypeptides can also be identified by Southern and/or PCR analysis and are useful in animal models for GPCR-related disorders. Knowledge of the sequence of a human GPCR DNA also makes possible, through use of Southern hybridization or polymerase chain reaction (PCR), the identification of genomic DNA sequences encoding GPCR expression control regulatory sequences such as promoters, operators, enhancers, repressors, and the like. Polynucleotides of the invention are also useful in hybridization assays to detect the capacity of cells to express GPCR polypeptides. Polynucleotides of the invention may also be the basis for diagnostic methods useful for identifying a genetic alteration(s) in a GPCR locus that underlies a disease state or states, which information is useful both for diagnosis and for selection of therapeutic strategies.

The disclosure herein of full length polynucleotides encoding GPCR polypeptides of the present invention makes readily available to the worker of ordinary skill in the art every possible fragment of the full length polynucleotides. The invention therefore provides fragments of GPCR-encoding polynucleotides comprising at least 14-15, and preferably at least 18, 20, 25, 50, or 75 consecutive nucleotides of a polynucleotide encoding GPCR polypeptides. Preferably, fragment polynucleotides of the invention comprise sequences unique to the GPCR-encoding polynucleotide sequence, and therefore hybridize under highly stringent or moderately stringent conditions only (i.e., “specifically”) to polynucleotides encoding GPCR polypeptides (or fragments thereof). Polynucleotide fragments of genomic sequences of the invention comprise not only sequences unique to the coding region, but also include fragments of the full length sequence derived from introns, regulatory regions, and/or other non-translated sequences. Sequences unique to polynucleotides of the invention are recognizable through sequence comparison to other known polynucleotides, and can be identified through use of alignment programs routinely utilized in the art, e.g., those made available in public sequence databases. Such sequences also are recognizable from Southern and Northern hybridization analyses to determine the number of fragments of genomic DNA and RNA to which a polynucleotide will hybridize. Polynucleotides of the invention can be labeled in a manner that permits their detection, including radioactive, fluorescent, and enzymatic labeling.

Fragment polynucleotides are particularly useful as probes for detection of full length or other fragment GPCR polynucleotides. One or more fragment polynucleotides can be included in kits that are used to detect the presence of a polynucleotide encoding a GPCR polypeptide, or used to detect variations in a polynucleotide sequences encoding GPCR polypeptides.

The invention also embraces DNAs encoding GPCR polypeptides which DNAs hybridize under moderately stringent or high stringency conditions to the non-coding strand, or complement, of the polynucleotide in any one of SEQ ID NOS: 1, 3, 5, 7, 9, 11,13, 15, 17 or 19.

Exemplary highly stringent hybridization conditions are as follows: hybridization at 42° C. in a hybridization solution comprising 50% formamide, 1% SDS, 1 M NaCl, 10% Dextran sulfate, and washing twice for 30 minutes at 60° C. in a wash solution comprising 0.1×SSC and 1% SDS. It is understood in the art that conditions of equivalent stringency can be achieved through variation of temperature and buffer, or salt concentration as described Ausubel, et al. (Eds.), Protocols in Molecular Biology, John Wiley & Sons (1994), pp.6.0.3 to 6.4.10. Modifications in hybridization conditions can be empirically determined or precisely calculated based on the length and the percentage of guanosine/cytosine (GC) base pairing of the probe. The hybridization conditions can be calculated as described in Sambrook et al., (Eds.), Molecular Cloning: A Laboratory Manual, Cold Spring Harbor Laboratory Press: Cold Spring Harbor, N.Y. (1989), pp. 9.47 to 9.51.

Autonomously replicating recombinant expression constructs such as plasmid and viral DNA vectors incorporating polynucleotides of the invention are also provided. Expression constructs wherein GPCR-encoding polynucleotides are operatively linked to an endogenous or exogenous expression control DNA sequence and a transcription terminator are also provided. Expression control DNA sequences include promoters, enhancers, and operators, and are generally selected based on the expression systems in which the expression construct is to be utilized. Preferred promoter and enhancer sequences are generally selected for the ability to increase gene expression, while operator sequences are generally selected for the ability to regulate gene expression. Expression constructs of the invention may also include sequences encoding one or more selectable markers that permit identification of host cells bearing the construct. Expression constructs may also include sequences that facilitate, and preferably promote, homologous recombination in a host cell. Preferred constructs of the invention also include sequences necessary for replication in a host cell.

Expression constructs are preferably utilized for production of an encoded protein, but also may be utilized simply to amplify GPCR-encoding polynucleotide sequences.

According to another aspect of the invention, host cells are provided, including prokaryotic and eukaryotic cells, comprising a polynucleotide of the invention (or vector of the invention) in a manner which permits expression of the encoded GPCR polypeptide. Polynucleotides of the invention may be introduced into the host cell as part of a circular plasmid, or as linear DNA comprising an isolated protein coding region or a viral vector. Methods for introducing DNA into the host cell well known and routinely practiced in the art include transformation, transfection, electroporation, nuclear injection, or fusion with carriers such as liposomes, micelles, ghost cells, and protoplasts. Expression systems of the invention include bacterial, yeast, fungal, plant, insect, invertebrate, and mammalian cells systems.

Host cells of the invention are a valuable source of immunogen for development of antibodies specifically immunoreactive with GPCR polypeptides. Host cells of the invention are also useful in methods for large scale production of GPCR polypeptides wherein the cells are grown in a suitable culture medium and the desired polypeptide products are isolated from the cells or from the medium in which the cells are grown by purification methods known in the art, e.g., conventional chromatographic methods including immunoaffinity chromatography, receptor affinity chromatography. hydrophobic interaction chromatography, lectin affinity chromatography, size exclusion filtration, cation or anion exchange chromatography. high pressure liquid chromatography (HPLC), reverse phase HPLC, and the like. Still other methods of purification include those wherein the desired protein is expressed and purified as a fusion protein having a specific tag, label, or chelating moiety that is recognized by a specific binding partner or agent. The purified protein can be cleaved to yield the desired protein, or be left as an intact fusion protein. Cleavage of the fusion component may produce a form of the desired protein having additional amino acid residues as a result of the cleavage process.

Knowledge of GPCR DNA sequences allows for modification of cells to permit, or increase, expression of endogenous GPCR. Cells can be modified (e.g., by homologous recombination) to provide increased expression by replacing, in whole or in part, the naturally occurring GPCR promoter with all or part of a heterologous promoter so that the cells express GPCR polypeptides at higher levels. The heterologous promoter is inserted in such a manner that it is operatively linked to endogenous GPCR polypeptide encoding sequences. [See, for example, PCT International Publication No. WO 94/12650, PCT International Publication No. WO. 92/20808, and PCT International Publication No. WO 91/09955.] It is also contemplated that, in addition to heterologous promoter DNA, amplifiable marker DNA (e.g., ada, dhfr, and the multifunctional CAD gene which encodes carbamyl phosphate synthase, aspartate transcarbamylase, and dihydroorotase) and/or intron DNA may be inserted along with the heterologous promoter DNA. If linked to the GPCR coding sequence, amplification of the marker DNA by standard selection methods results in co-amplification of the GPCR coding sequences in the cells.

The DNA sequence information provided by the present invention also makes possible the development through, e.g. homologous recombination or “knock-out” strategies [Capecchi, Science 244: 1288-1292 (1989)], of animals that fail to express functional GPCR polypeptides or that express a variant of GPCR polypeptides. Such animals (especially small laboratory animals such as rats, rabbits, and mice) are useful as models for studying the in vivo activities of GPCR polypeptides and modulators of GPCR polypeptides.

Also made available by the invention are anti-sense polynucleotides which recognize and hybridize to polynucleotides encoding GPCR polypeptides. Full length and fragment anti-sense polynucleotides are provided. Fragment anti-sense molecules of the invention include those which specifically recognize and hybridize to GPCR RNA (as determined by sequence comparison of DNA encoding GPCR polypeptides to DNA encoding other known molecules). Identification of sequences unique to GPCR-encoding polynucleotides, can be deduced through use of any publicly available sequence database, and/or through use of commercially available sequence comparison programs. The uniqueness of selected sequences in an entire genome can be further verified by hybridization analyses. After identification of the desired sequences, isolation through restriction digestion or amplification using any of the various polymerase chain reaction techniques well known in the art can be performed. Antisense polynucleotides are particularly relevant to regulating expression of GPCR polypeptides by those cells expressing GPCR mRNA.

Antisense nucleic acids (preferably 10 to 20 base pair oligonucleotides) capable of specifically binding to GPCR expression control sequences or GPCR RNA are introduced into cells (e.g., by a viral vector or colloidal dispersion system such as a liposome). The antisense nucleic acid binds to the GPCR target nucleotide sequence in the cell and prevents transcription or translation of the target sequence.

Phosphorothioate and methylphosphonate antisense oligonucleotides are specifically contemplated for therapeutic use by the invention. The antisense oligonucleotides may be further modified by poly-L-lysine, transferrin polylysine, or cholesterol moieties at their 5′ end. Suppression of GPCR polypeptide expression at either the transcriptional or translational level is useful to general cellular and/or animal models for diseases characterized by aberrant expression. Suppression of GPCR polypeptide expression at either the transcriptional or translational level is useful to generate cellular animal models for diseases characterized by aberrant GPCR polypeptide expression.

The GPCR polynucleotide and polypeptide sequences taught in the present invention facilitate the design of novel transcription factors for modulating GPCR polypeptide expression in native cells and animals, and cells transformed or transfected with GPCR polynucleotides. For example, the Cys₂-His₂ zinc finger proteins, which bind DNA via their zinc finger domains, have been shown to be amenable to structural changes that lead to the recognition of different target sequences. These artificial zinc finger proteins recognize specific target sites with high affinity and low dissociation constants, and are able to act as gene switches to modulate gene expression. Knowledge of the particular GPCR target sequence of the present invention facilitates the engineering of zinc finger proteins specific for the target sequence using known methods such as a combination of structure-based modeling and screening of phage display libraries [Segal et al., Proc Natl Acad Sci USA 96: 2758-2763 (1999); Liu et al., Proc Natl Acad Sci USA 94: 5525-30 (1997); Greisman and Pabo Science 275: 657-61 (1997); Choo et al., J Mol Biol 273: 525-32 (1997)]. Each zinc finger domain usually recognizes three or more base pairs. Since a recognition sequence of 18 base pairs is generally sufficient in length to render it unique in any known genome, a zinc finger protein consisting of 6 tandem repeats of zinc fingers would be expected to ensure specificity for a particular sequence [Segal et al., Proc Natl Acad Sci USA 96: 2758-2763 (1999)]. The artificial zinc finger repeats, designed based on GPCR polynucleotide sequences, are fused to activation or repression domains to promote or suppress GPCR polypeptides expression [Liu et al., Proc Natl Acad Sci USA 94: 5525-30 (1997)]. Alternatively, the zinc finger domains can be fused to the TATA box-binding factor (TBP) with varying lengths of linker region between the zinc finger peptide and the TBP to create either transcriptional activators or repressors [Kim et al., Proc Natl Acad Sci USA 94: 3616-3620 (1997)]. Such proteins, and polynucleotides that encode them, have utility for modulating GPCR polypeptide expression in vivo in both native cells, animals and humans; and/or cells transfected with GPCR polynucleotide-encoding sequences. The novel transcription factor can be delivered to the target cells by transfecting constructs that express the transcription factor (gene therapy), or by introducing the protein. Engineered zinc finger proteins can also be designed to bind RNA sequences for use in therapeutics as alternatives to antisense or catalytic RNA methods [McColl et al., Proc Natl Acad Sci USA 96:9521-6 (1999); Wu et al., Proc Natl Acad Sci USA 92:344-348 (1995)]. The present invention contemplates methods of designing such transcription factors based on the gene sequence of the invention, as well as customized zinc finger proteins, that are useful to modulate GPCR polypeptide expression in cells (native or transformed) whose genetic complement includes these sequences.

The invention also provides purified and isolated mammalian GPCR polypeptides encoded by a polynucleotide of the invention. Presently preferred is a human GPCR polypeptide comprising the amino acid sequence set out in any one of SEQ ID NOS: 2, 4, 6, 8, 10, 12, 14, 16, 18 or 20.

The invention also embraces polypeptides that have at least 99%, at least 95%, at least 90%, at least 85%, at least 80%, at least 75%, at least 70%, at least 65%, at least 60%, at least 55% or at least 50% identity and/or homology to a preferred polypeptide of the invention. Percent amino acid sequence “identity” with respect to the preferred polypeptide of the invention is defined herein as the percentage of amino acid residues in the candidate sequence that are identical with the residues in a GPCR polypeptide sequence after aligning both sequences and introducing gaps, if necessary, to achieve the maximum percent sequence identity, and not considering any conservative substitutions as part of the sequence identity. Percent sequence “homology” with respect to the preferred polypeptide of the invention is defined herein as the percentage of amino acid residues in the candidate sequence that are identical with the residues in a GPCR sequence after aligning the sequences and introducing gaps, if necessary, to achieve the maximum percent sequence identity, and also considering any conservative substitutions as part of the sequence identity.

In one aspect, percent homology is calculated as the percentage of amino acid residues in the smaller of two sequences which align with identical amino acid residue in the sequence being compared, when four gaps in a length of 100 amino acids may be introduced to maximize alignment [Dayhoff, in Atlas of Protein Sequence and Structure, Vol. 5, p. 124, National Biochemical Research Foundation, Washington, D.C. (1972), incorporated herein by reference].

Polypeptides of the invention may be isolated from natural cell sources or may be chemically synthesized, but are preferably produced by recombinant procedures involving host cells of the invention. Use of mammalian host cells is expected to provide for such post-translational modifications (e.g., glycosylation, truncation, lipidation, and phosphorylation) as may be needed to confer optimal biological activity on recombinant expression products of the invention. Glycosylated and non-glycosylated forms of GPCR polypeptides are embraced.

The invention also embraces variant (or analog) GPCR polypeptides. In one example, insertion variants are provided wherein one or more amino acid residues supplement a GPCR amino acid sequence. Insertions may be located at either or both termini of the protein, or may be positioned within internal regions of the GPCR amino acid sequence. Insertional variants with additional residues at either or both termini can include for example, fusion proteins and proteins including amino acid tags or labels.

Insertion variants include GPCR polypeptides wherein one or more amino acid residues are added to a GPCR amino acid sequence, or to a biologically active fragment thereof.

Variant products of the invention also include mature GPCR polypeptide products, i.e., GPCR polypeptide products wherein leader or signal sequences are removed, with additional amino terminal residues. The additional amino terminal residues may be derived from another protein, or may include one or more residues that are not identifiable as being derived from a specific proteins. GPCR polypeptide products with an additional methionine residue at position −1 (Met⁻¹-GPCR) are contemplated, as are variants with additional methionine and lysine residues at positions −2 and −1 (Met⁻²-Lys⁻¹-GPCR). Variants of GPCR polypeptide with additional Met, Met-Lys, Lys residues (or one or more basic residues in general) are particularly useful for enhanced recombinant protein production in bacterial host cell.

The invention also embraces GPCR polypeptide variants having additional amino acid residues which result from use of specific expression systems. For example, use of commercially available vectors that express a desired polypeptide as part of glutathione-S-transferase (GST) fusion product provides the desired polypeptide having an additional glycine residue at position −1 after cleavage of the GST component from the desired polypeptide. Variants which result from expression in other vector systems are also contemplated.

Insertional variants also include fusion proteins wherein the amino and/or carboxy termini of a GPCR polypeptide is fused to another polypeptide.

In another aspect, the invention provides deletion variants wherein one or more amino acid residues in a GPCR polypeptide are removed. Deletions can be effected at one or both termini of the GPCR polypeptide, or with removal of one or more residues within the GPCR amino acid sequence. Deletion variants, therefore, include all fragments of a GPCR polypeptide.

The invention also embraces polypeptide fragments of the sequence set out in SEQ ID NO: 2 wherein the fragments maintain biological (e.g., ligand binding and/or intracellular signaling) or immunological properties of a GPCR polypeptide. Fragments comprising at least 5, 10, 15, 20, 25, 30, 35, or 40 consecutive amino acids of SEQ ID NO: 2 are comprehended by the invention. Preferred polypeptide fragments display antigenic properties unique to or specific for human GPCR and its allelic and species homologs. Fragments of the invention having the desired biological and immunological properties can be prepared by any of the methods well known and routinely practiced in the art.

In still another aspect, the invention provides substitution variants of GPCR polypeptides. Substitution variants include those polypeptides wherein one or more amino acid residues of a GPCR polypeptide are removed and replaced with alternative residues. In one aspect, the substitutions are conservative in nature, however, the invention embraces substitutions that are also non-conservative. Conservative substitutions for this purpose may be defined as set out in Tables A, B, or C below.

Variant polypeptides include those wherein conservative substitutions have been introduced by modification of polynucleotides encoding polypeptides of the invention. Amino acids can be classified according to physical properties and contribution to secondary and tertiary protein structure. A conservative substitution is recognized in the art as a substitution of one amino acid for another amino acid that has similar properties. Exemplary conservative substitutions are set out in Table A (from WO 97/09433, page 10, published Mar. 13, 1997 (PCT/GB96/02197, filed Sep. 6, 1996), immediately below.

TABLE A
Conservative Substitutions I

| SIDE CHAIN | |
|---|---|
| CHARACTERISTIC | AMINO ACID |
| Aliphatic | |
| Non-polar | GA P I L V |
| Polar - uncharged | C S T M N Q |
| Polar - charged | D E K R |
| Aromatic | H F W Y |
| Other | N Q D E |

Alternatively, conservative amino acids can be grouped as described in Lehninger, [Biochemistry, Second Edition; Worth Publishers, Inc. NY:N.Y. (1975), pp.71-77] as set out in Table B, immediately below.

TABLE B
Conservative Substitutions II

| SIDE CHAIN | |
|---|---|
| CHARACTERISTIC | AMINO ACID |
| Non-polar (hydrophobic) | |
| A. Aliphatic: | A L I V P |
| B. Aromatic: | F W |
| C. Sulfur-containing: | M |
| D. Borderline: | G |
| Uncharged-polar | |
| A. Hydroxyl: | S T Y |
| B. Amides: | N Q |
| C. Sulfhydryl: | C |
| D. Borderline: | G |
| Positively Charged (Basic): | K R H |
| Negatively Charged (Acidic): | DE |

As still an another alternative, exemplary conservative substitutions are set out in Table C, immediately below.

TABLE C
Conservative Substitutions III

| Original | |
|---|---|
| Residue | Exemplary Substitution |
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| Ile (I) | Leu, Val, Met, Ala, Phe, |
| Leu (L) | Ile, Val, Met, Ala, Phe |
| Lys (K) | Arg, Gln, Asn |
| Met (M) | Leu, Phe, Ile |
| Phe (F) | Leu, Val, Ile, Ala |
| Pro (P) | Gly |
| Ser (S) | Thr |
| Thr (T) | Ser |
| Trp (W) | Tyr |
| Tyr (Y) | Trp, Phe, Thr, Ser |
| Val (V) | Ile, Leu, Met, Phe, Ala |

GPCR polypeptide variants that display ligand binding properties of native GPCR polypeptides and are expressed at higher levels, and variants that provide for constitutive active receptor are particularly useful in assays of the invention. Such variants also are useful in cellular and animal models for diseases characterized by aberrant GPCR polypeptide expression/activity.

It should be understood that the definition of polypeptides of the invention is intended to include polypeptides bearing modifications other than insertion, deletion, or substitution of amino acid residues. By way of example, the modifications may be covalent in nature, and include for example, chemical bonding with polymers, lipids, other organic, and inorganic moieties. Such derivatives may be prepared to increase circulating half-life of a polypeptide, or may be designed to improve targeting capacity for the polypeptide to desired cells, tissues, or organs.

Similarly, the invention further embraces GPCR polypeptides that have been covalently modified to include one or more water soluble polymer attachments such as polyethylene glycol, polyoxyethylene glycol, or polypropylene glycol.

In a related embodiment, the present invention provides compositions comprising purified polypeptides of the invention. Preferred compositions comprise, in addition to the polypeptide of the invention, a pharmaceutically acceptable (i.e., sterile and non-toxic) liquid, semisolid, or solid diluents that serve as pharmaceutical vehicles, excipients, or media. Any diluent known in the art may be used. Exemplary diluents include, but are not limited to, water, saline solutions, polyoxyethylene sorbitan monolaurate, magnesium stearate, methyl- and propylhydroxybenzoate, talc, alginates, starches, lactose, sucrose, dextrose, sorbitol, mannitol, glycerol, calcium phosphate, mineral oil, and cocoa butter.

Also comprehended by the present invention are antibodies (e.g., monoclonal and polyclonal antibodies, single chain antibodies, chimeric antibodies, bifunctional/bispecific antibodies, humanized antibodies, human antibodies, and complementary determining region (CDR)-grafted antibodies, including compounds which include CDR sequences which specifically recognize a polypeptide of the invention) specific for GPCR polypeptides of the invention or fragments thereof. Preferred antibodies of the invention are human antibodies which can be produced and identified according to methods described in WO93/11236, published Jun. 20, 1993, which is incorporated herein by reference in its entirety. Antibody fragments, including Fab, Fab′, F(ab′)₂, and Fᵥ, are also provided by the invention. The term “specific for,” when used to describe antibodies of the invention, indicates that the variable regions of the antibodies of the invention recognize and bind GPCR polypeptides exclusively (i.e., able to distinguish GPCR polypeptides from other known GPCR polypeptides by virtue of measurable differences in binding affinity, despite the possible existence of localized sequence identity, homology, or similarity between GPCR polypeptides and such polypeptides). It will be understood that specific antibodies may also interact with other proteins (for example, S. aureus protein A or other antibodies in ELISA techniques) through interactions with sequences outside the variable region of the antibodies, and in particular, in the constant region of the molecule. Screening assays to determine binding specificity of an antibody of the invention are well known and routinely practiced in the art. For a comprehensive discussion of such assays, see Harlow et al. (Eds), Antibodies A Laboratory Manual; Cold Spring Harbor Laboratory; Cold Spring Harbor, N.Y. (1988), Chapter 6. Antibodies that recognize and bind fragments of the GPCR polypeptides of the invention are also contemplated, provided that the antibodies are, first and foremost, specific for GPCR polypeptides. Antibodies of the invention can be produced using any method well known and routinely practiced in the art.

Non-human antibodies may be humanized by any methods known in the art. In one method, the non-human CDRs are inserted into a human antibody or consensus antibody framework sequence. Further changes can then be introduced into the antibody framework to modulate affinity or immunogenicity.

Antibodies of the invention are useful for, for example, therapeutic purposes (by modulating activity of GPCR polypeptides), diagnostic purposes to detect or quantitate GPCR polypeptides, as well as purification of GPCR polypeptides. Kits comprising an antibody of the invention for any of the purposes described herein are also comprehended. In general, a kit of the invention also includes a control antigen for which the antibody is immunospecific.

Specific binding molecules, including natural ligands and synthetic compounds, can be identified or developed using isolated or recombinant GPCR polypeptide products, GPCR polypeptide variants, or preferably, cells expressing such products. Binding partners are useful for purifying GPCR polypeptide products and detection or quantification of GPCR polypeptide products in fluid and tissue samples using known immunological procedures. Binding molecules are also manifestly useful in modulating (i.e., blocking, inhibiting or stimulating) biological activities of GPCR polypeptides, especially those activities involved in signal transduction.

The DNA and amino acid sequence information provided by the present invention also makes possible identification of binding partner compounds with which a GPCR polypeptide or polynucleotide will interact. Methods to identify binding partner compounds include solution assays, in vitro assays wherein GPCR polypeptides are immobilized, and cell based assays. Identification of binding partner compounds of GPCR polypeptides provides candidates for therapeutic or prophylactic intervention in pathologies associated with GPCR polypeptide normal and aberrant biological activity.

The invention includes several assay systems for identifying GPCR polypeptide binding partners. In solution assays, methods of the invention comprise the steps of (a) contacting a GPCR polypeptide with one or more candidate binding partner compounds and (b) identifying the compounds that bind to the GPCR polypeptide. Identification of the compounds that bind the GPCR polypeptide can be achieved by isolating the GPCR polypeptide/binding partner complex, and separating the GPCR polypeptide from the binding partner compound. An additional step of characterizing the physical, biological, and/or biochemical properties of the binding partner compound is also comprehended in another embodiment of the invention. In one aspect, the GPCR polypeptide/binding partner complex is isolated using a antibody immunospecific for either the GPCR polypeptide or the candidate binding partner compound.

In still other embodiments, either the GPCR polypeptide or the candidate binding partner compound comprises a label or tag that facilitates its isolation, and methods of the invention to identify binding partner compounds include a step of isolating the GPCR polypeptide/binding partner complex through interaction with the label or tag. An exemplary tag of this type is a poly-histidine sequence, generally around six histidine residues, that permits isolation of a compound so labeled using nickel chelation. Other labels and tags, such as the FLAG tag (Eastman Kodak, Rochester, N.Y.), well known and routinely used in the art, are embraced by the invention.

In one variation of an in vitro assay, the invention provides a method comprising the steps of (a) contacting an immobilized GPCR polypeptide with a candidate binding partner compound and (b) detecting binding of the candidate compound to GPCR polypeptide. In an alternative embodiment, the candidate binding partner compound is immobilized and binding of GPCR polypeptide is detected. Immobilization is accomplished using any of the methods well known in the art, including covalent bonding to a support, a bead, or a chromatographic resin, as well as non-covalent, high affinity interaction such as antibody binding, or use of streptavidin/biotin binding wherein the immobilized compound includes a biotin moiety. Detection of binding can be accomplished (i) using a radioactive label on the compound that is not immobilized, (ii) using a fluorescent label on the nonimmobilized compound, (iii) using an antibody immunospecific for the non-immobilized compound, (iv) using a label on the non-immobilized compound that excites a fluorescent support to which the immobilized compound is attached, as well as other techniques well known and routinely practiced in the art.

The invention also provides cell-based assays to identify binding partner compounds of a GPCR polypeptide. In one embodiment, the invention provides a method comprising the steps of contacting a GPCR polypeptide expressed on the surface of a cell with a candidate binding partner compound and detecting binding of the candidate binding partner compound to the GPCR polypeptide. In a preferred embodiment, the detection comprises detecting a calcium flux or other physiological cellular events caused by the binding of the molecule.

Agents that modulate (i.e., increase, decrease, or block) GPCR polypeptide activity or expression may be identified by incubating a putative modulator with a cell expressing a GPCR polypeptide or polynucleotide and determining the effect of the putative modulator on GPCR polypeptide activity or expression. The selectivity of a compound that modulates the activity of GPCR polypeptides can be evaluated by comparing its effects on GPCR polypeptides to its effect on other G coupled-protein receptor compounds. Selective modulators may include, for example. antibodies and other proteins, peptides, or organic molecules which specifically bind to a G coupled-protein receptor polypeptide or a G coupled-protein receptor-encoding nucleic acid. Modulators of GPCR polypeptide activity will be therapeutically useful in treatment of diseases and physiological conditions in which normal or aberrant GPCR polypeptide activity is involved.

Methods of the invention to identify modulators include variations on any of the methods described above to identify binding partner compounds, the variations including techniques wherein a binding partner compound has been identified and the binding assay is carried out in the presence and absence of a candidate modulator. A modulator is identified in those instances where binding between the GPCR polypeptide and the binding partner compound changes in the presence of the candidate modulator compared to binding in the absence of the candidate modulator compound. A modulator that increases binding between the GPCR polypeptide and the binding partner compound is described as an enhancer or activator, and a modulator that decreases binding between the GPCR polypeptide and the binding partner compound is described as an inhibitor.

The invention also comprehends high throughput screening (HTS) assays to identify compounds that interact with or inhibit biological activity (i.e., inhibit enzymatic activity, binding activity, etc.) of a GPCR polypeptide. HTS assays permit screening of large numbers of compounds in an efficient manner. Cell-based HTS systems are contemplated to investigate GPCR receptor-ligand interaction. HTS assays are designed to identify “hits” or “lead compounds” having the desired property, from which modifications can be designed to improve the desired property. Chemical modification of the “hit” or “lead compound” is often based on an identifiable structure/activity relationship between the “hit” and the GPCR polypeptide.

Mutations in the GPCR gene that result in loss of normal function of the GPCR gene product underlie GPCR polypeptide-related human disease states. The invention comprehends gene therapy to restore activity to treat those disease states. Delivery of a functional GPCR gene to appropriate cells is effected ex vivo, in situ, or in vivo by use of vectors, and more particularly viral vectors (e.g., adenovirus, adeno-associated virus, or a retrovirus), or ex vivo by use of physical DNA transfer methods (e.g., liposomes or chemical treatments). See, for example, Anderson, Nature, supplement to vol. 392, no. 6679, pp.25-20 (1998). For additional reviews of gene therapy technology see Friedmann, Science, 244: 1275-1281 (1989), Verma, Scientific American: 68-84 (1990); and Miller, Nature, 357: 455-460 (1992). Alternatively, it is contemplated that in other human disease states, preventing the expression of or inhibiting the activity of GPCR polypeptides of the invention will be useful in treating the disease states. It is contemplated that antisense therapy or gene therapy could be applied to negatively regulate the expression of GPCR polypeptides of the invention.

Additional features of the invention will be apparent from the following Examples.

### EXAMPLE 1

### Cloning of G Protein-Coupled Receptors

The Incyte and Genbank expressed sequence tag (EST) databases were searched with the NCBI program Blastall using either the transmembrane VI region of known dopamine receptors (leading to the identification of CON193, CON166, CON103 and CON203) or all known GPCR's except olfactory and opsin receptors (leading to the identification of CON198, CON197, CON202, CON222, CON215) as query sequences, to find patterns suggestive of novel G protein-coupled receptors. Positive hits from the find-pattern program were further analyzed with the GCG program BLAST to determine which ones were the most likely candidates to encode a GPCR, using the standard (default) alignment produced by BLAST as a guide.

A. Cloning of CON193 G Protein-Coupled Receptor

A.1. Database Search Results

Searching identified Clone 3091220H1 in the Incyte database as an interesting candidate sequence. The 3091220H1 Clone was obtained and sequenced directly using an AB1377 fluorescence-based sequencer (Perkin-Elmer/Applied Biosystems Division, PE/ABD, Foster City, Calif.) and the ABI PRISM™ Ready Dye-Deoxy Terminator kit with Taq FS™ polymerase. Each ABI cycle sequencing reaction contained about 0.5 μg of plasmid DNA. Cycle-sequencing was performed using an initial denaturation at 98° C. for 1 minute, followed by 50 cycles using the following parameters: 98° C. for 30 seconds, annealing at 50° C. for 30 seconds, and extension at 60° C. for 4 minutes. Temperature cycles and times were controlled by a Perkin-Elmer 9600 thermocycler. Extension products were purified using Centriflex™ gel filtration cartridges (Advanced Genetic Technologies Corp., Gaithersburg, Md.). Each reaction product was loaded by pipette onto the column, which was then centrifuged in a swinging bucket centrifuge (Sorvall model RT6000B tabletop centrifuge) at 1500×g for 4 minutes at room temperature. Column-purified samples were dried under vacuum for about 40 minutes and then dissolved in 5 μl of a DNA loading solution (83% deionized formamide, 8.3 mM EDTA, and 1.6 mg/ml Blue Dextran). The samples were then heated to 90° C. for three minutes and loaded into the gel sample wells for sequence analysis using the ABI377 sequencer. Sequence analysis was done by importing ABI377 files into the Sequencer program (Gene Codes, Ann Arbor, Mich.). Generally, sequence reads of 700 bp were obtained. Potential sequencing errors were minimized by obtaining sequence information from both DNA strands and by re-sequencing difficult areas using primers annealing at different locations until all sequencing ambiguities were removed.

From the sequence it was deduced that Clone 3091220H1 contained only an amino-terminal fragment of a putative GPCR corresponding to the third through the seventh transmembrane regions (3TM-7TM) of a GPCR. Referring to SEQ ID NO: 1, the nucleotide sequence of Clone 3091220H1 corresponds to nucleotides 404 to 1308 of what was eventually determined to be the complete sequence of a novel seven-transmembrane receptor designated CON193. A database search with this partial sequence showed a 56% match to members of the olfactory receptor gene family, e.g., the gene encoding mouse odorant receptor S19.

A.2 Screening of a Genomic Phage Library to Obtain a Full-Length GPCR Clone:

The PCR technique was used to prepare a genomic fragment for use as a probe specific for the genomic CON193 Clone. Based on the complete sequence of Clone 3091220H1, two oligonucleotide primers were designed: Primer LW1282: 5′-TAATACCTGCACTGCCCAC-3′ (SEQ ID NO: 21; see nucleotides 876-894 of SEQ ID NO: 1) and Primer LW1283: 5′-TCTTTCCTTCTCTTCTCACTCC-3′ (SEQ ID NO: 22 see nucleotides 1137-1158 of SEQ ID NO:1). These primers were designed to amplify a 283 base-pair fragment of genomic DNA containing a portion of the CON193 coding region found in Clone 3091220H1 (assuming the absence of introns in this region).

Initially, a suitable human genomic library constructed in EMBL3 SP6/T7 (Clontech Laboratories) was amplified to provide the materials required for screening. Two microliters of the human genomic library (approximately 10⁸ plaque-forming units per milliliter; Clontech Laboratories, catalog number HL1067J) were added to 6 ml of an overnight culture of K802 cells (Clontech Laboratories), and 250 μl aliquots were distributed into each of 24 tubes. The tubes were incubated at 37° C. for 15 minutes, and then 7 ml of 0.8% agarose (i.e., top agarose) at 50° C. were added to each tube. After mixing, the contents of the tubes were poured onto 150 mm LB plates and incubated overnight at 37° C. to allow clone amplification, evident as plaque formation (typically, confluent lysis was observed rather than discrete plaques). To each plate, 5 ml of SM phage buffer (0.1 M NaCl, 8.1 μM MgSO₄.7H₂O, 50 mM Tris-HCl (pH 7.5), and 0.0001% gelatin) was added and the top agarose was removed by scraping with a microscope slide. Top agarose slurries containing phage were then placed in individual 50 ml centrifuge tubes. A drop of chloroform was added and each tube was placed in a 37° C. shaker for 15 minutes, followed by centrifuging at 2,750×g for 15 minutes. The supernatants were isolated and separately stored at 4° C. as 24 stock solutions of amplified library clones.

As noted above, polymerase chain reaction (PCR) was selected as a technique for screening the phage library. Each PCR reaction was done in a 20 μl reaction volume containing 8.84 μl H₂O, 2 μl 10×PCR buffer II (Perkin-Elmer), 2 μl 25 mM MgCl₂, 0.8 μl dNTP mixture (dATP, dCTP, dGTP, dCTP, each at 10 mM), 0.12 μl primer LW1282 (approximately 1 μg/μl), 0.12 μl primer LW1283 (approximately 1 μg/μl), 0.12 μl AmpliTaq Gold polymerase (5 Units/μl, with “Units” as defined by the supplier, Perkin-Elmer) and 2 μl of phage from one of the 24 stock tubes. The PCR reaction involved 1 cycle at 95° C. for 10 minutes and 80° C. for 20 minutes, followed by 22 cycles at 95° C. for 30 seconds, 72-51° C. for 2 minutes (72° C. for this stage of the second cycle, with a decrease of one degree for this stage in each succeeding cycle). 72° C. for one minute, followed by 30 cycles at 95° C. for 15 seconds, 50° C. for 30 seconds, and 72° C. for one minute.

Following PCR cycling, the contents from each reaction tube were loaded onto a 2% agarose gel and electrophoresed adjacent to known size standards to screen for PCR products of the expected size, indicative of a clone containing the 283 bp portion of Clone 3091220H1 amplified by the two selected primers. A positive signal (i.e., a fragment of the expected size) was found in one of the 24 PCR reactions, thereby identifying a single stock genomic library tube containing positive clones.

From the original genomic library tube that had given a PCR product of the correct size, a 5 μl phage aliquot was used to establish a set of five serial dilutions (1/100, v/v) that were plated and incubated in the same manner as described for the amplification of the phage library. Following incubation, BA85 nitrocellulose filters (Schleicher & Schuell) were placed on top of each of the plates for 1 hour to adsorb phage from the plaques that had formed in the top agarose during incubation.

Each filter was then gently removed, placed phage side up in an individual petri dish, and covered with 4 ml of SM buffer for 15 minutes to elute the phage. One milliliter of SM containing eluted phage was removed from each plate and used to set up a PCR reaction as described above. The plate containing the most dilute phage solution to yield a PCR product of the expected size was then subdivided using the following procedure. A BA85 filter was placed on the top agar of the plate and the medium with applied filter was physically divided into 24 sections. After one hour to allow phage adsorption to the 24 filters, each filter was removed and separately incubated in 1 ml of SM buffer at room temperature for 15 minutes. Two microliters of each eluted phage solution were then used as a PCR substrate. Those plate sections yielding positive PCR results were then subdivided into 12 subsections by removing the top agar and incubating it in 200 μl of SM buffer for one hour at room temperature. Again, 2 μl of the eluted phage solutions were plated and lifted using BA85 filters, and PCR reactions were repeated. The procedure for progressive dilution of phage was continued until a single plaque was isolated. Subsequently, 10 μl of eluted phage from that single plaque were added to 100 μl SM and 200 μl of K802 cells for plating in a single petri dish as described above. A total of 7 plates were inoculated in this manner. Following incubation at 37° C. for 16 hours. the top agarose from each of the 7 plates was removed to recover the phage, which were used to prepare purified genomic phage DNA using the Qiagen Lambda Midi Kit.

The purified CON193 genomic phage DNA was sequenced using the ABI PRISM™ 310 Genetic Analyzer (Perkin-Elmer/Applied Biosystems) which uses advanced capillary electrophoresis technology and the ABI PRISM™ BigDye™ Terminator Cycle Sequencing Ready Reaction Kit. The cycle-sequencing reaction contained 18 μl of H₂O, 16 μl of BigDye™ Terminator mix, 3 μl of genomic phage DNA (0.26 μg/μl), and 3 μl primer (25 ng/μl). The reaction was performed in a Perkin-Elmer 9600 thermocycler at 95° C. for 5 minutes, followed by 75 cycles of 95° C. for 30 seconds, 55° C. for 20 seconds, and 60° C. for 4 minutes. The final subclone was also sequenced using the ABI PRISM™ 310 Genetic Analyzer. The cycle-sequencing reaction contained 6 μl of H₂O, 8 μl of BigDye™ Terminator mix, 5 μl of miniprep clone DNA (0.1 μg/μl), and 1 μl primer (25 ng/μl). The reaction was performed in a Perkin-Elmer 9600 thermocycler at 25 cycles of 96° C. for 10 seconds, 50° C. for 10 seconds, and 60° C. for 4 minutes. The product of the PCR reaction was purified using Centriflex T gel filtration cartridges, dried under vacuum, and dissolved in 16 μl of Template Suppression Reagent (PE-Applied Biosystems). The samples were then incubated at 95° C. for 5 minutes and placed in the 310 Genetic Analyzer. These efforts resulted in the determination of the CON193 polynucleotide sequence set forth in SEQ ID NO:1 and the deduced amino acid sequence of the encoded CON193 polypeptide which is set forth in SEQ ID NO:2.

A.3 Subcloning of the Coding Region of CON193 via PCR

Additional experiments were conducted to subclone the coding region of CON193 and place the isolated coding region into a useful vector. Two additional PCR primers were designed based on the coding region of CON193. The first PCR primer, designated Primer LW1373, has the sequence 5′-GCATAAGCTTATGCTAACACTGAATAAAACAG-3′ (SEQ ID NO: 23), nucleotides 11-32 of which correspond to nucleotides 157-178 of SEQ ID NO: 1. The second PCR primer is Primer LW1374, which has the sequence 5′-GCATCTCGAGTCACATGCTGTAGGATTTGG-3′ (SEQ ID NO: 24, nucleotides 11-30 of which correspond to the complement of nucleotides 1102-1121 of SEQ ID NO: 1. To protect against exonucleolytic attack during subsequent exposure to enzymes, e.g., Taq polymerase, primers were routinely synthesized with a protective run of nucleotides at the 5′ end that were not necessarily complementary to the desired target.

PCR was performed in a 50 μl reaction containing 35 μl H₂O, 5 μl 10× TT buffer (140 mM ammonium sulfate, 0.1% gelatin, 0.6 M Tris-tricine, pH 8.4), 5 μl 15 mM MgSO₄, 2 μl dNTP mixture (dGTP, dATP, dTTP, and dCTP, each at 10 mM), 2 μl genomic phage DNA (0.26 μg/μl), 0.3 μl Primer LW1373 (1 μg/μl), 0.3 μl Primer LW1374 (1 μg/μl), 0.4 μl High Fidelity Taq polymerase (Boehringer Mannheim). The PCR reaction was started with 1 cycle of 94° C. for 2 minutes; followed by 15 cycles at 94° C. for 30 seconds, 55° C. for 30 seconds, and 72° C. for 1.3 minutes.

The contents from the PCR reaction were loaded onto a 2% agarose gel, fractionated and electroeluted. The DNA band of expected size was excised from the gel, placed in a GenElute Agarose spin column (Supelco) and spun for 10 minutes at maximum speed in a microcentrifuge. The eluted DNA was precipitated with ethanol and resuspended in 6 μl H₂O for ligation.

The PCR-amplified DNA fragment containing the CON193 coding region was cloned into pCR2.1 using a protocol standard in the art. In particular, the ligation reaction consisted of 6 μl of CON193 DNA, 1 μl 10× ligation buffer, 2 μl pCR2.1 (25 ng/μl, Invitrogen), and 1 μl T4 DNA ligase (Invitrogen). The reaction mixture was incubated overnight at 14° C. and the reaction was then stopped by heating at 65° C. for 10 minutes. Two microliters of the ligation reaction were transformed into One Shot cells (Invitrogen) and plated onto ampicillin plates. A single colony containing an insert was used to inoculate a 5 ml culture of LB medium. The culture was grown for 18 hours and the plasmid DNA was purified using the Concert Rapid Plasmid Miniprep System (GibcoBRL) and sequenced. Following confirmation of the sequence, pCR-CON193 was identified, and a 50 ml culture of LB medium was inoculated and recombinant plasmid DNA was purified using a Qiagen Plasmid Midi Kit to yield purified pCR-CON193.

B. Cloning of CON166 G Protein-Coupled Receptor

B.1 Database Search Results

The database searching identified clone 2553280H1 in the Incyte database as an interesting candidate sequence. The 2553280H1 clone was obtained and sequenced directly using an ABI377 fluorescence-based sequencer and the ABI PRISM™ Ready Dye-Deoxy Terminator kit with Taq FS™ polymerase as described above for CON193 in Example 1A.1. From the sequence it was deduced that clone 2553280H1 contained 349 nucleotides of a GPCR coding region comprising a carboxy-terminal fragment of a putative GPCR corresponding to the sixth and seventh transmembrane regions (6TM and 7TM). In addition, clone 2553280H1 contained 1.2 kb of the 3′ untranslated sequence of that GPCR. Referring to SEQ ID NO: 3, the nucleotide sequence of Clone 2553280H1 corresponds to nucleotides 663 to 1,014 of what was eventually determined to be the complete sequence of a novel seven-transmembrane receptor that was designated CON166. A database search with this partial sequence showed a 44% match to an activated T cell-specific G protein-coupled receptor.

B2. Screening of a Genomic Phage Library to Obtain a Full-Length GPCR Clone

The PCR technique was used to prepare a genomic fragment for use as a probe specific for the genomic CON166 clone. Based on the complete sequence of clone 2553280H1, two oligonucleotide primers were designed: Primer LW1278: 5′-ACCGCTGCCTTTTTAGTC-3′ (SEQ ID NO: 28; see nucleotides 715 to 732 of SEQ ID NO: 3 and Primer LW1279: 5′-CCTTCTTTCTGGGTACATAAGTC-3′ (SEQ ID NO: 29; see the reverse complement of nucleotides 951-973 of SEQ ID NO: 3). These primers were designed to amplify a 259 base-pair fragment of genomic DNA containing a portion of the CON166 coding region found in clone 2553280H1 (assuming the absence of introns in this region).

Initially, a suitable human genomic library constructed in EMBL SP6/T7 was amplified to provide the materials required for screening as described above for CON193 in Example 1A.2. Polymerase chain reaction (PCR) was selected as a technique for screening the phage library. Each PCR reaction was done in a 20 μl reaction volume containing 8.84 μl H₂O, 2 μl 10× PCR buffer II (Perkin-Elmer), 2 μl 25 mM MgCl₂, 0.8 μl dNTP mixture (dATP, dCTP, dGTP, dCTP, each at 10 mM), 0.12 μl primer LW1278 (approximately 1 μg/μl), 0.12 μl primer LW1279 (approximately 1 μg/μl), 0.12 μl AmpliTaq Gold polymerase (5 Units/μl, with “Units” as defined by the supplier, Perkin-Elmer) and 2 μl of phage from one of the 24 stock tubes. The PCR reaction involved 1 cycle at 95° C. for 10 minutes and 80° C. for 20 minutes, followed by 12 cycles at 95° C. for 30 seconds, 72-61° C. for 2 minutes (72° C. for this stage of the second cycle, with a decrease of one degree for this stage in each succeeding cycle), 72° C. for 30 seconds, followed by 30 cycles at 95° C. for 15 seconds, 60° C. for 30 seconds, and 72° C. for 30 seconds.

Following PCR cycling, the contents from each reaction tube were loaded onto a 2% agarose gel and electrophoresed adjacent to known size standards to screen for PCR products of the expected size of 259 bp, indicative of a clone containing the portion of clone 2553280H1 amplified by the two selected primers. A positive signal (i.e., a fragment of the expected size) was found in one of the 24 PCR reactions, thereby identifying a single stock genomic library tube containing positive clones.

From the original genomic library tube that had given a PCR product of the correct size, a 5 μl phage aliquot was used to amplify the CON166 genomic phage DNA as described for CON193 above in Example 1A.2. For the amplification of the phage library, the plates containing the diluted phage solution were subdivided into 12 sections unlike that of CON193; otherwise the procedures were identical.

The purified CON166 genomic phage DNA was sequenced using the ABI PRISM™ 310 Genetic Analyzer which uses advanced capillary electrophoresis technology and the ABI PRISM™ BigDye™ Terminator Cycle Sequencing Ready Reaction Kit as described above for CON193 in Example 1A.2. These efforts resulted in the determination of the CON166 polynucleotide sequence set forth in SEQ ID NO: 3 and the deduced amino acid sequence of the encoded CON166 polypeptide which is set forth in SEQ ID NO: 4.

B.3 Subcloning of the Coding Region of CON166 Via PCR

Additional experiments were conducted to subclone the coding region of CON166 from the genomic clone and place the isolated coding region into a useful vector. Two additional PCR primers were designed based on the coding region of CON166. The first PCR primer. designated Primer LW1405, has the sequence 5′ -AAGCATAACATGGATGAAACAGGAAATCTG-3′ (SEQ ID NO: 29, nucleotides 10-30 of which correspond to nucleotides 1-21 of SEQ ID NO: 3). To protect against exonucleolytic attack during subsequent exposure to enzymes. e.g., Taq polymerase, primers were routinely synthesized with a protective run of nucleotides at the 5′ end that were not necessarily complementary to the desired target. The second PCR primer is Primer LW1406, which has the sequence 5′-AAGCATAACTATACTTTACATATTTCTTC-3′ (SEQ ID NO: 30, nucleotides 9-29 of which correspond to the reverse complement of nucleotides 994-1014 of SEQ ID NO: 3).

PCR was performed in a 50 ul reaction containing 34 μl H₂O, 5 μl 10× TT buffer (140 mM ammonium sulfate, 0.1% gelatin, 0.6 M Tris-tricine, pH 8.4), 5 μl 15 mM MgSO₄, 2 μl dNTP mixture (dGTP, dATP, dTTP, and dCTP, each at 10 mM), 3 μl genomic phage DNA (0.25 μg/μl), 0.3 μl Primer LW1405 (1 μg/μl), 0.3 μl Primer LW1406 (1 μg/μl), 0.4 μl High Fidelity Taq polymerase (Boehringer Mannheim). The PCR reaction was started with 1 cycle of 94° C. for 2 minutes; followed by 25 cycles at 94° C. for 30 seconds, 55° C. for 30 seconds, and 72° C. for 1.3 minutes.

The contents from the PCR reaction were loaded onto a 2% agarose gel and fractionated. The DNA band of expected size (1,031 bp) was excised from the gel, placed in a GenElute Agarose spin column (Supelco) and spun for 10 minutes at maximum speed in a microfuge. The eluted DNA was precipitated with ethanol and resuspended in 6 μl H₂O for ligation.

The PCR-amplified DNA fragment containing the CON166 coding region was cloned into pCR2.1 to generate pCR-CON166 using a protocol standard in the art. In particular, the ligation reaction was carried out as described for CON193 in Example 1A.3. The resulting plasmid DNA was purified using the Concert Rapid Plasmid Miniprep System (GibcoBRL) and sequenced. Following confirmation of the sequence, a 50 ml culture of LB medium was inoculated with the transformed One Shot cells, cultured, and processed using a Qiagen Plasmid Midi Kit to yield purified pCR-CON166.

C. Cloning of CON103 G Protein-Coupled Receptor

C.1 Database Search Results

The database searching identified clone 1581220H1 in the Incyte database as an interesting candidate sequence. The 1581220H1 clone was obtained and sequenced directly using an AB1377 fluorescence-based sequencer and the ABI PRISM™ Ready Dye-Deoxy Terminator kit with Taq FS™ polymerase as described for CON193 in Example 1A.1. From the sequence it was deduced that clone 1581220H1 contained 454 nucleotides of a GPCR coding region comprising a carboxy-terminal fragment of a putative GPCR corresponding to the sixth and seventh transmembrane regions (6TM and 7TM). In addition, clone 1581220H1 contained 1.2 kb of the 3′ untranslated sequence of that GPCR. Referring to SEQ ID NO: 5, the nucleotide sequence of clone 1581220H1 corresponds to nucleotides 698 to 1190 of what was eventually determined to be the complete sequence of a novel seventransmembrane receptor designated CON103. A database search with this partial sequence showed a 44% match to an activated T cell-specific G protein-coupled receptor.

C.2 Screening of a Genomic Phage Library to Obtain a Full-Length GPCR Clone

The PCR technique was used to prepare a genomic fragment for use as a probe specific for the genomic CON103 clone. Based on the complete sequence of clone 1581220H1, two oligonucleotide primers were designed: Primer LW1280: 5′-TCTGCACACAGCTCTTCCATGG-3′ (SEQ ID NO: 32; see nucleotides 1568-1589 of SEQ ID NO: 5) and Primer LW1281: 5′-TCCCTTGTCCAGTTGGTTGAGG-3′ (SEQ ID NO: 33; see nucleotides 1926 to 1947 of SEQ ID NO: 5. These primers were designed to amplify a 380 base-pair fragment of genomic DNA containing a portion of the CON103 coding region found in clone 1581220H1 (assuming the absence of introns in this region).

Initially, a suitable human genomic library constructed in EMBL SP6/T7 was amplified to provide the materials required for screening as described above for CON193 in Example 1A.2. Polymerase chain reaction (PCR) was selected as a technique for screening the phage library. Each PCR reaction was done in a 20 μl reaction volume containing 8.84 μl H₂O, 2 μl 10× PCR buffer 11 (Perkin-Elmer), 2 μl 25 mM MgCl₂, 0.8 μl dNTP mixture (dATP, dTTP, dGTP, dCTP, each at 10 mM), 0.12 μl primer LW1280 (approximately 1 μlg/μl), 0.12 μl primer LW1281 (approximately 1 μg/μl), 0.12 μl AmpliTaq Gold polymerase (5 Units/μl, with “Units” as defined by the supplier, Perkin-Elmer) and 2 μl of phage from one of the 24 stock tubes. PCR amplification reactions using each one of the other 23 stock collections of genomic clones were performed under the same conditions. The PCR reaction involved 1 cycle at 95° C. for 10 minutes and 80° C. for 20 minutes, followed by 12 cycles at 95° C. for 30 seconds, 72-61° C. for 2 minutes (72° C. for this stage of the second cycle, with a decrease of one degree for this stage in each succeeding cycle), 72° C. for one minute, followed by 30 cycles at 95° C. for 15 seconds, 60° C. for 30 seconds, and 72° C. for 30 seconds.

Following PCR cycling, the contents from each reaction tube were loaded onto a 2% agarose gel and electrophoresed adjacent to known size standards to screen for PCR products of the expected size of 380 bp, indicative of a clone containing the portion of clone 1581220H1 amplified by the two selected primers. A positive signal (i.e., a fragment of the expected size) was found in one of the 24 PCR reactions, thereby identifying a single stock genomic library tube containing positive clones.

From the original genomic library tube that had given a PCR product of the correct size, a 5 μl phage aliquot was used to amplify the CON103 genomic phage DNA as described above for CON193 in Example 1A.2. A total of 8 plates were inoculated with eluted phage in this manner described above. Following incubation at 37° C. for 16 hours, the top agarose from each of the 8 plates was removed to recover the phage, which were used to prepare purified genomic phage DNA using the Qiagen Lambda Midi Kit.

The CON103 clone was sequenced using the ABI PRISM™ 310 Genetic Analyzer. The cycle-sequencing reaction contained 6 μl of H₂O, 8 μl of BigDye™ Terminator mix, 5 μl of miniprep clone DNA (0.1 μg/μl). and 1 μl primer (25 ng/μl). The reaction was performed in a Perkin-Elmer 9600 thermocycler at 25 cycles of 96° C. for 10 seconds, 50° C. for 10 seconds, and 60° C. for 4 minutes. The product of the PCR reaction was purified using Centriflex™ gel filtration cartridges, dried under vacuum, and dissolved in 16 μl of Template Suppression Reagent (PE-Applied Biosystems). The samples were then incubated at 95° C. for 5 minutes and placed in the 310 Genetic Analyzer. These efforts resulted in the determination of the CON103 polynucleotide sequence set forth in SEQ ID NO: 5 and the deduced amino acid sequence of the encoded CON103 polypeptide which is set forth in SEQ ID NO: 6.

C.3 Subcloning of the Coding Region of CON103 via PCR

Additional experiments were conducted to subclone the coding region of CON103 from the genomic clone and place the isolated coding region into a useful vector. Two additional PCR primers were designed based on the sequence of the coding region of CON103: Primer LW1385 (5′-GCATAAGCTTCCATGGAACTTCATAACCTG-3′; SEQ ID NO: 34, nucleotides 13-30 of which correspond to nucleotides 1-18 of SEQ ID NO: 5) and Primer LW1386 (5′-GCATCTCGAGTTACCCCCACAGCGCTGCAG-3′; SEQ ID NO: 35, nucleotides 11-30 of which correspond to the reverse complement of nucleotides 1171-1190 of SEQ ID NO: 5). To protect against exonucleolytic attack during subsequent exposure to enzymes, e.g., Taq polymerase, primers were routinely synthesized with a protective run of nucleotides at the 5′ end that were not necessarily complementary to the desired target.

PCR was performed in a 50 μl reaction containing 22.6 μl H₂O, 5 μl 10× TT buffer (140 mM ammonium sulfate, 0.1% gelatin, 0.6 M Tris-tricine, pH 8.4), 5 μl 15 mM MgSO₄, 10 μl rapid dye (Origene), 2 μl dNTP mixture (dGTP, dATP, dTTP, and dCTP, each at 10 mM), 0.5 μl genomic phage DNA (0.97 μg/μl), 0.3 μl Primer LW1385 (1 μl/μl), 0.3 μl Primer LW1386 (1 μg/μl), and 0.4 μl High Fidelity Taq polymerase (Boehringer Mannheim). The PCR reaction was started with 1 cycle of 94° C. for 2 minutes. followed by 12 cycles at 94° C. for 30 seconds, 55° C. for 30 seconds, and 72° C. for 1.3 minutes.

The contents from the PCR reaction were loaded onto a 2% agarose gel and fractionated. The DNA band of expected size (1,212 bp) was excised from the gel, placed in a GenElute Agarose spin column (Supelco) and spun for 10 minutes at maximum speed in a microcentrifuge. The eluted DNA was precipitated with ethanol and resuspended in 6 μl H₂O for ligation.

The PCR-amplified DNA fragment containing the CON103 coding region was cloned into pCR2.1 using a protocol standard in the art. In particular, the ligation reaction was carried out as described above for CON193 in Example 1A.3. The resulting plasmid DNA was purified using the Concert Rapid Plasmid Miniprep System (GibcoBRL) and sequenced. Following confirmation of the sequence, pCRCON103 was identified, and a 50 ml culture of LB medium was inoculated, cultured, and processed using a Qiagen Plasmid Midi Kit to yield purified pCR-CON103.

D. Cloning of CON203 G Protein-Coupled Receptor

D.1 Database Search Results

The database searching identified clone 3210396H1 in the Incyte database as an interesting candidate sequence. The 3210396H1 clone was obtained and sequenced directly using an ABI377 fluorescence-based sequencer and the ABI PRISM™ Ready Dye-Deoxy Terminator kit with Taq FS™ polymerase as described above for CON193 in Example 1A.1. From the sequence it was deduced that clone 3210396H1 contained all 1,002 nucleotides of a GPCR coding region (see SEQ ID NO: 7). A database search with this sequence showed a 33% match to a platelet activating receptor (Gene H963, GenBank Acc. No. AF002986).

D.2 Subcloning of the Coding Region of CON203 via PCR

Additional experiments were conducted to subclone the coding region of CON203 and place the isolated coding region into a useful vector. Two additional PCR primers were designed based on the sequence of the coding region of CON203: Primer LW1329: 5′-GCATCTCGAGTCAGCCTAAGGTTATGTTG-3′ (SEQ ID NO: 36; see nucleotides 984 to 1,002 of SEQ ID NO: 7 for the reverse complement of nucleotides 9-29 of SEQ ID NO: 36) and Primer LW1377: 5′-GCATAAGCTTATGAACACCACAGTGATGC-3′ (SEQ ID NO: 37; see nucleotides 1-19 of SEQ ID NO: 7 which correspond to nucleotides 11-29 of SEQ ID NO: 37). To protect against exonucleolytic attack during subsequent exposure to enzymes, e.g., Taq polymerase, primers were routinely synthesized with a protective run of nucleotides at the 5′ end that were not necessarily complementary to the desired target. These primers were designed to amplify a 1,020 base-pair fragment of clone 3210396H1 containing the complete coding region of CON203.

PCR was performed in a 50 μl reaction containing 34 μl H₂O, 5 μl 10× TT buffer (140 mM ammonium sulfate, 0.1% gelatin, 0.6 M Tris-tricine, pH 8.4), 5 μl 15 MM MgSO₄, 2 μl dNTP mixture (dGTP, dATP, dTTP, and dCTP, each at 10 mM), 3 μl clone 3210396H1 (miniprep DNA), 0.3 μl Primer LW1329 (1 μg/μl), 0.3 μl Primer LW1377 (1 μg/μl), and 0.4 μl High Fidelity Taq polymerase (Boehringer Mannheim). The PCR reaction was started with 1 cycle of 94° C. for 2 minutes, followed by 12 cycles at 94° C. for 30 seconds, 55° C. for 30 seconds, and 72° C. for 1.3 minutes.

The contents from the PCR reaction were loaded onto a 1.2% agarose gel and fractionated. The DNA band of expected size (1,020 bp) was excised from the gel, placed in a GenElute Agarose spin column (Supelco) and spun for 10 minutes at maximum speed in a microcentrifuge. The eluted DNA was precipitated with ethanol and resuspended in 6 μl H₂O for ligation.

The PCR-amplified DNA fragment containing the CON203 coding region was cloned into pCR2.1 using a standard protocol and the Original TA Cloning Kit (Invitrogen). Ligation reactions were carried out as described above for CON193 in Example 1A.3. The resulting plasmid DNA was purified using the Concert Rapid Plasmid Miniprep System (GibcoBRL) and sequenced. Following confirmation of the sequence, pCR-C203 was identified, and a 50 ml culture of LB medium was inoculated, cultured, and processed using a Qiagen Plasmid Midi Kit to yield purified pCR-C203.

The CON203 clone was sequenced using the ABI PRISM™ 310 Genetic Analyzer (P-E Applied Biosystems), which uses advanced capillary electrophoresis technology and the ABI Prism™ BigDye™ Terminator Cycle Sequencing Ready Reaction Kit. The cycle-sequencing reaction contained 6 μl of H₂O, 8 μl of BigDye™ Terminator mix, 5 μl of miniprep clone DNA (0.1 μg/μl), and 1 μl primer (25 ng/μl). The reaction was performed in a Perkin-Elmer 9600 thermocycler using the following conditions: 25 cycles of 96° C. for 10 seconds, 50° C. for 10 seconds, and 60° C. for 4 minutes. The product of the PCR reaction was purified using Centriflex™ gel filtration cartridges, dried under vacuum, and dissolved in 16 μl of Template Suppression Reagent (PE-Applied Biosystems). The samples were then incubated at 95° C. for 5 minutes and placed in the 310 Genetic Analyzer.

Initially, these efforts showed that the CON203 coding region cloned into pCR2.1 had a single bp difference from the corresponding sequence of clone 3210396H1. The single bp change in the pCR2.1 clone was eliminated by conforming that sequence to the sequence of clone 3210396H1 using the QuikChange Site-Directed Mutagenesis Kit (Stratagene). The method involves modification of a sequence during PCR amplification, for which PCR primers LW1387 (5′-GAGAAATATTTTTCTAAAAAAACCTGTTTTTGCAAAAACGG-3′; SEQ ID NO: 38) and LW1388 (5′-CCGTTTTTGCAAAAACAGGTTTTTTTAGAAAAATATTTCTC-3′; SEQ ID NO: 39) were used. The PCR reaction contained 40 μl H₂O, 5 μl 10× proprietary Reaction Buffer (Stratagene), 1 μl pCR-C203 (0.125 μl/μl) mini-prep DNA, 1 μl dNTP mixture (dGTP, dATP, dTTP, and dCTP, each at 10 mM), 1 μl Pfu DNA polymerase (2.5 Units/μl), 1 μl LW1387 (125 ng/μl) and 1 μl LW1388 (125 ng/1). The cycle conditions were 95° C. for 30 seconds, followed by 12 cycles at 95° C. for 30 seconds, 55° C. for 1 minute, and 68° C. for 12 minutes. The tube was then placed on ice for 2 minutes and 1 μl of DpnI was added. The tube was then incubated at 37° C. for one hour. One microliter of the DpnI-treated DNA was transformed into Epicurian coli XL1-Blue supercompetent E. coli cells. Following isolation of pCR-C203, the entire insert was re-sequenced, thereby successfully verifying repair of the single-site polymorphism. As expected, the sequence of the CON203 coding region determined using this pCR2.1 clone is in complete agreement with the CON203 coding region sequence of SEQ ID NO: 7 which specifies the amino acid sequence set forth in SEQ ID NO: 8.

E. Cloning of CON198 G Protein-Coupled Receptor

E.1 Database Search Results

The database searching identified Clone 3359808H1 in the Incyte database as an interesting candidate sequence. The 3359808H1 clone was obtained and sequenced using standard techniques. From the sequence it was deduced that Clone 3359808H1 contained the entire coding region for a previously unidentified GPCR, which was designated “CON198.” The DNA and deduced amino acid sequences for CON198 are set forth in SEQ ID NOS: 9 and 10, respectively. A database search with this CON198 DNA sequence showed a 61% match to the rat putative GPCR designated RAIc [Raming et. al., Recept Channels, 6: 141-151 (1998)] and 46% identity to an olfactory receptor.

E.2 Subcloning of the Coding Region of CON198 via PCR

Additional experiments were conducted to subclone the coding region of the CON198 clone into a useful vector. Two PCR primers were designed based on the coding region of CON198 for the purpose of PCR amplification of the CON198 coding sequence. The first, Primer LW1326, from 5′ to 3′ (SEQ ID NO: 42): GCATGAATTCATGATGGTGGATCCCAATGG, includes the 5′ end of the CON198 coding sequence (underlined) as well as a EcoRI restriction site, useful for subsequent expression work. The second, Primer LW1327, from 5′ to 3′ (SEQ ID NO: 43): GCATCTCGAGCCTAGGGCTCTGAAGCG, includes sequence complementary to the 3′ end of the CON198 coding sequence (underlined), preceded by a XhoI restriction site sequence useful for subsequent cloning and expression work.

The PCR was performed in a 50 μl reaction containing 34 μl H₂O, 5 μl of 10× TT buffer (140 mM Ammonium Sulfate, 0.1% gelatin, 0.6 M Tris-tricine, pH 8.4), 5 μl of 15 mM MgSO₄, 2 μl of 10 mM dNTPs (dATP, dCTP, dTTP, dGTP), 2 μl of Clone 3359808H1 mini-prep DNA (approx. 0.125 μg/μl), 0.3 μl of Primer LW1326 (1 μg/μl), 0.3 μl of Primer LW1327 (1 μg/μl), and 0.5 μl of High Fidelity Taq polymerase (Boehringer Mannheim). The PCR reaction was started with 1 cycle of 94° C. for 2 minutes, followed by 12 cycles at 94° C. for 30 seconds. 55° C. for 30 seconds, and 72° C. for 1 minute.

The contents from the PCR reaction were loaded onto a 1.2% agarose gel and electrophoresed. The DNA band of expected size was excised from the gel, placed in a GenElute Agarose spin column (Supelco) and spun for 10 minutes at maximum speed in a microcentrifuge. The eluted DNA was ethanol-precipitated and resuspended in 6 μl H₂O for ligation.

The purified PCR fragment containing the CON198 coding sequence was ligated into a commercial vector using Invitrogen's Original TA Cloning Kit. The ligation reaction was carried out as described above for CON193 in Example 1A.3. The resulting plasmid DNA was isolated using a Concert Rapid Plasmid Miniprep System (GibcoBRL) and sequenced to confirm that the plasmid contained the CON198 insert. Sequencing of the subcloned CON198 construct revealed that the PCR amplification had introduced a mutation (relative to the sequence of the original clone) at the nucleotide corresponding to position 204 of SEQ ID NO: 9. A site-directed mutagenesis experiment was performed using the QuikChange Site-Directed Mutagenesis Kit (Stratagene) to repair the mutation.

Two primers were designed to revert the mutated A nucleotide at position 204 back to a G nucleotide via polymerase chain reaction. Primer LW1415 (SEQ ID NO: 44) contained the sequence: 5′-CCATGTATATATTTCTTTGCATGCTTTCAGGCATTGACATCC-3′; and primer LW1416 (SEQ ID NO: 45) contained the sequence: 5′-GGATGTCAATGCCTGAAAGCATGCAAAGAAATATATACATGG-3′. The PCR reaction contained 40 μl of H₂O, 5 μl of 10× Reaction buffer, 1 μl of mini-prep DNA (approx. 0.125 μg/μl) from the CON198-pCR2.1 clone (as template), 1 μl of primer LW1415 (125 ng/μl), 1 μl of primer LW1416 (125 ng/ μl), 1 μl of 10 mM dNTPs, 1 μl Pfu DNA polymerase. The PCR cycle conditions were as follows: initial denaturation at 95° C. for 30 seconds, then 14 cycles at 95° C. for 30 seconds, 55° C. annealing for 1 minute, and 68° C. extension for 12 minutes. Thereafter, the reaction tube was placed on ice for 2 minutes.

After PCR. 1 μl of DpnI was added and the tube incubated at 37° C. for one hour to digest the methylated parental DNA template. One microliter of the DpnI-treated DNA was transformed into Epicurian coli XL1-Blue supercompetent cells and the entire insert was re-sequenced. The resequencing confirmed that position 204 of SEQ ID NO: 9 had been successfully reverted to a guanine nucleotide.

Upon confirmation of the insert, the E. Coli transformant was used to inoculate a 50 ml culture of LB medium. The culture was grown for 16 hours at 37° C., and centrifuged into a cell pellet. Plasmid DNA was purified from the pellet using a Qiagen Plasmid Midi Kit and again sequenced to confirm successful cloning of the CON198 insert, using an ABI377 fluorescence-based sequencer and the ABI PRISM™ Ready Dye-Deoxy Terminator kit with Taq FS™ polymerase as described above for CON193 in Example 1A.1.

F. Cloning of CON197 G Protein-Coupled Receptor

F.1 Database Search Results

The database searching identified Clone 866390H1 in the Incyte database as an interesting candidate sequence. The 866390H1 clone was obtained and sequenced using standard techniques. From the sequence it was deduced that Clone 866390H1 contained the entire coding region for a previously unidentified GPCR, which was designated “CON197.” The DNA and deduced amino acid sequences for CON197 are set forth in SEQ ID NOs: 11 and 12, respectively. A database search with this CON197 DNA sequence showed a 42% match to an olfactory receptor.

F.2 Subcloning of the Coding Region of CON197 via PCR

Additional experiments were conducted to subclone the coding region of the CON197 clone into a useful vector. Two PCR primers were designed based on the coding region of CON197 for the purpose of PCR amplification of the CON197 coding sequence. The first, Primer LW1324, from 5′ to 3′ (SEQ ID NO: 48): GATCGGATCCATGGAAAGCGAGAACAG, includes the 5′ end of the CON197 coding sequence (underlined) as well as a BamHI restriction site, useful for subsequent expression work. The second, Primer LW1325, from 5′ to 3′ (SEQ ID NO: 49): GATCCTCGAGTCAGGCTATGTGCTTATTAAACACC, includes sequence complementary to the 3′ end of the CON197 coding sequence (underlined), preceded by a XhoI restriction site sequence useful for subsequent cloning and expression work.

The PCR was performed in a 50 μl reaction containing 24 μl H₂O, 10 μl Rapid Dye Loading buffer (Origene) 5 μl 10×TT buffer (140 mM Ammonium Sulfate, 0.1% gelatin, 0.6 M Tris-tricine, pH 8.4), 5 μl of 15 mM MgSO₄, 2 μl of 10 mM dNTPs (dATP, dCTP, dTTP, dGTP), 3 μl of Clone 866390H1 mini-prep DNA (approx. 0.125 μg/μl), 0.3 μl of Primer LW1324 (1 μg/μl), 0.3 μl of Primer LW1325 (1 μg/μl), and 0.5 μl of High Fidelity Taq polymerase (Boehringer Mannheim). The PCR reaction was started with 1 cycle of 94° C. for 2 minutes; followed by 12 cycles at 94° C. for 30 seconds, 55° C. for 30 seconds, and 72° C. for 1 minute.

The contents from the PCR reaction was loaded onto a 1.2% agarose gel and electrophoresed. The DNA band of expected size was excised from the gel, placed in GenElute Agarose spin column (Supelco) and spun for 10 minutes at maximum speed in a Savant microcentrifuge. The eluted DNA was ethanol-precipitated and resuspended in 6 μl H₂O for ligation.

The purified PCR fragment containing the CON197 coding sequence was ligated into a commercial vector using Invitrogen's Original TA Cloning Kit. The resulting plasmid DNA from the culture was isolated using a Concert Rapid Plasmid Miniprep System (GibcoBRL) and sequenced to confirm that the plasmid contained the CON197 insert.

Upon confirmation of the insert, the same transformant was used to inoculate a 50 ml culture of LB medium. The culture was grown for 16 hours at 37° C., and centrifuged into a cell pellet. Plasmid DNA was purified from the pellet using a Qiagen Plasmid Midi Kit and again sequenced to confirm successful cloning of the CON197 insert, using an ABI377 fluorescence-based sequencer (Perkin Elmer/Applied Biosystems Division, PE/ABD, Foster City, Calif.) and the ABI PRISM™ Ready Dye-Deoxy Terminator kit with Taq FS™ polymerase as described above for CON193 in Example 1A.1.

G. Cloning of CON202 G Protein-Coupled Receptor

G.1 Database Search Results

The database searching identified Clone Number 1305513H1 in the Incyte database as an interesting candidate sequence. The 1305513H1 clone was obtained and sequenced using an ABI377 fluorescence-based sequencer (Perkin Elmer/Applied Biosystems Division, PE/ABD, Foster City, Calif.) and the ABI PRISM™ Ready Dye-Deoxy Terminator kit with Taq FS™ polymerase as described above for CON193 in Example 1A.1.

Sequencing of Incyte Clone 1305513H1 revealed a sequence corresponding to nucleotides 1054 to 1378 of SEQ ID NO: 13. Using a FORTRAN computer program called “tmtrest.all” [Parodi et al., Comput. Appl. Biosci., 5: 527-535 (1994)], Clone 1305513H1 was deduced to contain two transmembrane-spanning domains (TMVI and TMVII) and an extracellular loop for a previously unidentified GPCR, which was designated as “CON202”. The sequence obtained was used as a tool to identify a full length GPCR clone as described in the next section.

G.2 PCR Screening of Genomic Clones

A human genomic phage library was selected as a source from which to attempt to clone the CON202 gene. The genomic library was amplified as described above for CON193 in Example 1A.2.

This genomic library was screened by PCR using the primers: GV599 (5′GGCAGAAGAAGGCTATTGGTCTTAGACGAG3′; SEQ ID NO: 52), and GV600 (5′CTGAAACAGCGCCTCAGCTCCC3′; SEQ ID NO: 53). These primers were designed from the sequence of Clone 1305513H1 to amplify a 253 base pair fragment (corresponding to nucleotides 1064 to 1317 of SEQ ID NO: 13) from any corresponding genomic clone in the library. The 20 μl PCR reactions each contained 12.8 μl of H₂O, 2 μl of 10× PCR buffer II (Perkin-Elmer), 2 μl of 25 mM MgCl₂, 0.8 μl of 10 mM dNTP's (dATP, dGTP, dCTP, dTTP), 0.12 μl of primer GV599 (1 μg/ml), 0.12 μl of primer GV600 (1 μg/ml), 0.2 μl AmpliTaq Gold polymerase (5 Units/μl, with “Units” as defined by the supplier, Perkin Elmer) and 2 μl of phage from one of the 24 tubes. The PCR reaction consisted of 1 cycle at 95° C. for 10 minutes; then 17 cycles at 95° C. for 20 seconds, 72° C. for 2 minutes decreasing 1° C. each cycle. 72° C. for 30 seconds followed by 30 cycles at 95° C. for 20 seconds, 55° C. for 30 seconds, and 72° C. for 30 seconds.

The PCR products were visualized on a 2% agarose gel. For those tubes which produced the correct sized band of 253 bp, five microliters from each original phage culture tube were used to amplify the CON202 genomic phage DNA as described above for CON193 in Example 1A.2.

The genomic DNA from the single phage isolate, was sequenced with the ABI PRISM™ 310 Genetic Analyzer (PE Applied Biosystems) which uses advanced capillary electrophoresis technology and the ABI PRISM™ Big Dye™ Terminator Cycle Sequencing Ready Reaction Kit. The cycle-sequencing reaction contained 20 ml of H₂O, 16ml of BigDye™ Terminator Mix, 1 ml of genomic phage DNA (1.1 mg/ml), and 3 ml primer (25 ng/ml). The reaction was performed in a Perkin-Elmer 9600 thermocycler at 95° C. for 5 minutes, followed by 99 cycles of 95° C. for 30 seconds, 55° C. for 20 seconds and 60° C. for 4 minutes. The product was purified using a Centriflex™ gel filtration cartridge, dried under a vacuum, then dissolved in 16 ml of Template Suppression Reagent. The samples were heated at 95° C. for 5 minutes then placed in the 310 Genetic Analyzer.

G.3 Subcloning of the Coding Region of CON202 via PCR

Additional experiments were conducted to subclone the coding region of the CON202 clone into a more useful vector. Two PCR primers were designed based on the coding region of CON202 for the purpose of PCR amplification of the CON202 coding sequence. The first, Primer LW1482 (5′AGCTATGGCGAACTATAGCCATGCAGC3′; SEQ ID NO: 54) included the 5′ end of the CON202 coding sequence (underlined). The second, Primer LW148 (5′AGTCCTCATATAACACAGTAAGGTTCC3′; SEQ ID NO: 55) included the sequence complementary to the 3′ end of the CON202 coding sequence (underlined).

The PCR was performed in a 50 μl reaction containing 36.5 μl of H₂O, 5 μl of 10× TT buffer (140 mM Ammonium Sulfate, 0.1% gelatin, 0.6 M Tris-tricine, pH 8.4), 5 μl of 15 mM MgSO₄, 2 μl of 10 mM dNTP's (dATP, dCTP, dTTP, dGTP), 0.5 μl of CON202 genomic phage DNA (approx. 1.1 μg/μl), 0.3 l of Primer LW1482 (1 μg/μl), 0.3 μl of Primer LW1483 (1 μg/μl), and 0.4 μl of High Fidelity Taq polymerase (Boehringer Mannheim). The PCR reaction was started with 1 cycle of 94° C. for 2 minutes; followed by 12 cycles at 94° C. for 30 seconds, 55° C. for 30 seconds, and 72° C. for 1.3 minutes.

The contents from the PCR reaction were loaded onto a 2.1% agarose gel and electrophoresed. The DNA band of expected size (1.1 kb) was excised from the gel, placed on a GenElute Agarose spin column (Supelco), and spun for 10 minutes at maximum speed in a microfuge. The eluted DNA was ethanol-precipitated and resuspended in 6 μl of H₂O for ligation.

The purified PCR fragment, containing the CON202 coding sequence, was ligated into a commercial vector using Invitrogen's Original TA Cloning Kit. The ligation reaction was carried out as described above for CON193 in Example 1A.3. The resulting plasmid DNA from the culture was isolated using a Concert Rapid Plasmid Miniprep System (GibcoBRL) and sequenced to confirm that the plasmid contained the CON202 insert. The resulting construct was denoted as pCR-CON202.

The final subclone was sequenced using the ABI PRISM™ 310 Genetic Analyzer (PE Applied Biosystems) which uses advanced capillary electrophoresis technology and the ABI PRISM™ Terminator Cycle Sequencing Ready Reaction Kit. The cycle-sequencing reaction contained 6 ml of H₂O, 8 ml of BigDye™ Terminator mix, 5 ml miniprep DNA (0.1 mg/ml), and 1 ml primer (25 ng/ml). The reaction was performed in a Perkin-Elmer 9600 thermocycler at 25 cycles of 96° C. for 10 seconds, 50° C. for 10 seconds, and 60° C. for 4 minutes. The product was purified using Centriflex™ gel filtration cartridges, dried under vacuum, then dissolved in 16 ml of Template Suppression Reagent. The samples were heated to 95° C. for 5 minutes then placed in the 310 Genetic Analyzer.

Upon confirmation of the insert, the same transformant was used to inoculate a 50 ml culture of LB medium. The culture was grown for 16 hours at 37° C., and centrifuged into a cell pellet. Plasmid DNA was purified from the pellet using a Qiagen Plasmid Midi Kit and again sequenced to confirm successful cloning of the CON202 insert, as described above.

H. Cloning of CON222 G Protein-Coupled Receptor

H.1 Database Search Results

The database searching in the Incyte database identified Sequence Number 2488822CB1 as an interesting candidate sequence. This Incyte sequence is a consensus sequence derived by compiling multiple, shorter contiguous (apparently overlapping) partial sequences from cDNA clones. A single clone known to contain the complete consensus sequence was not available from Incyte. The following experiments were performed to clone a piece of human DNA which corresponds to the region of the theoretical Incyte Sequence Number 2488822CB that was deduced to encode a heretofore undescribed GPCR. The human DNA and protein that was eventually isolated is referred to herein as CON222.

H.2 Isolation of CON222 Genomic DNA Using PCR

To isolate a clone of CON222, PCR primers were designed based on the 5′ and 3′ ends of the open reading frame that was identified in the Incyte Sequence Number 2488822CB1. The first primer, designated as LW1440, has the sequence 5′AAGCGGATGTTTAGACCTCTTGTG3′ (SEQ ID NO: 60) which corresponds to nucleotides 1 to 18 of SEQ ID NO: 15 (underlined). The second primer, designated LW1441, has the sequence 5′AACAGTCATGAATAGGAATTGAG3′ (SEQ ID NO: 61) which is the reverse complement of nucleotides 1173 to 1191 of SEQ ID NO: 15 (underlined).

PCR was performed in a 50 ml reaction containing 22.1 ml H₂O, 10 ml Rapid Dye Loading Buffer (Origene), 5 ml 10×TT buffer (140 mM Ammonium Sulfate, 0.1% gelatin, 0.6 M Tris-tricine pH 8.4), 5 ml 15 mM MgSO₄, 2 ml 10 mM dNTP's (dATP, dCTP, dGTP, dTTP), 5 ml human genomic DNA (0.03 mg/ml) (Clontech, Cat# 6550-1), 0.3 ml of Primer LW1440 (1 mg/ml) (SEQ ID NO: 59), 0.3 ml of LW1441 (1 mg/ml) (SEQ ID NO: 60), 0.4 ml High Fidelity Taq polymerase (Boehringer Mannheim). The PCR reaction was started with 1 cycle of 94° C. for 2 minutes followed by 10 cycles at 94° C. for 30 seconds, 55° C. for 2 minutes, 72° C. for 2 minutes then 25 cycles at 94° C. for 30 seconds, 55° C. for 30 seconds, and 72° C. for 2 minutes. The PCR reaction was loaded onto a 1.2% agarose gel. The resulting band was not 1.2 kB in length as expected, indicating that this method was unsuccessful in identifying an appropriate clone from the selected Clontech genomic DNA library containing the coding region of CON222.

A human genomic DNA phage library was selected as an alternate source from which to attempt to clone the CON222 gene. Internal primers were designed to attempt to isolate from a genomic library a single phage which expresses the complete coding region. The procedure was carried out as described above for CON193 in Example 1A.2.

PCR was performed to identify a phage that contained a genomic DNA insert which corresponds to the deduced complete coding region of Incyte Sequence Number 2488822CB1 using the primers: Primer LW1442: 5′GCCATTCTGTCCACAGAAG3′ (SEQ ID NO: 58; see nucleotides 391 to 410 of SEQ ID NO: 15) and Primer LW1443: 5′TCAGTTGCTGTTATGGCAC3′ (SEQ ID NO: 59; see reverse complement of nucleotides 744 to 761 of SEQ ID NO: 15). These primers were designed based on the deduced coding region of Incyte Sequence Number 2488822CB1, to amplify a 370 bp fragment (corresponding to nucleotides 391 to 761 of SEQ ID NO: 1) from any corresponding genomic clone in the library. The 50 μl PCR reactions each contained 32 μl of H₂O, 5 μl of 10× PCR gold buffer (PE Applied Biosystems), 5 μl of 25 mM MgCl₂, 2 μl of 10 mM dNTP's (DATP, dCTP, dGTP, dTTP), 0.3 μl of primer LW1442 (1 μg/ml), 0.3 μl of primer LW1443 (1 μg/ml), 0.4 μl AmpliTaq Gold polymerase (5 U/μl, with “Units” defined by the supplier; PE Applied Biosystems) and 5 μl of phage isolated human genomic DNA (0.03 μg/μl). The PCR reaction consisted of 1 cycle at 95° C. for 10 minutes, then 17 cycles at 95° C. for 20 seconds and 72° C. for 2 minutes decreasing 1 degree each cycle, and 72° C. for 1 minute, followed by 30 cycles at 95° C. for 20 seconds, 55° C. for 30 seconds, and 72° C. for 1 minute. An aliquot of the PCR reaction was loaded onto a 1.2% agarose gel and electrophoresed. Although the internal primers were designed to produce a 370 bp PCR fragment, the resulting band was approximately 1.4 kb in length.

The DNA band was excised from the gel, placed on GenElute Agarose spin columns (Supelco) and spun for 10 minutes at maximum speed in a microcentrifuge. The eluted DNA was ethanol-precipitated and resuspended in 10 μl of H₂O and 5 μl was used to sequence the PCR band.

The PCR fragment was sequenced with an ABI PRISM™ 310 Genetic Analyzer (PE Applied Biosystems) which uses advanced capillary electrophoresis technology and the ABI PRISM™ BigDye™ Terminator Cycle Sequencing Ready Reaction Kit. Each cycle-sequencing reaction contained 6 ml of H₂O, 8 ml of BigDye Terminator mix, 5 ml PCR fragment DNA (0.2 mg/ml), and 1 ml Primer LW1442 (25 ng/ml) and Primer LW1443 (25 ng/ml). The reaction was performed in a Perkin-Elmer 9600 thermocycler with 25 cycles of 96° C. for 10 seconds, 50° C. for 10 seconds, and 60° C. for 4 minutes. The product was purified using Centriflex™ gel Reagent (PE Applied Biosystems). The samples were heated at 95° C. for 5 minutes then placed in the 310 Genetic Analyzer.

The sequence analysis determined that there is an intron in the middle of the 5th transmembrane-spanning domain between nucleotides 673 and 674 in SEQ ID NO: 15. This intron was responsible for the unexpectedly large PCR fragment.

H.3 Isolation of Full Length cDNA

Since attempts to isolate an uninterrupted coding region from genomic DNA were unsuccessful, a fetal brain cDNA was used to generate the complete coding region of Incyte Sequence Number 2488833CB1. The PCR primers described above, LW1440 (SEQ ID NO: 60) and LW1441 (SEQ ID NO: 61), which correspond to the 5′ and 3′ end of CON222 respectively, were used to generate the full length coding region.

The 50 μl PCR reaction contained 37.4 μl of H₂O, 5 μl of 10× cDNA PCR buffer (Clontech), 1 μl of 10 mM dNTP's (dATP, dCTP, dTTP, dGTP), 5 μl of Marathon-Ready Fetal Brain cDNA (Clontech), 0.3 μl of Primer LW1440 (1 μg/μl), 0.3 μl of Primer LW1441 (1 μg/μl), and 1 μl of 50× Advantage cDNA polymerase (Clontech). The PCR reaction was started with 1 cycle of 94° C. for 1 minute, followed by 30 cycles at 94° C. for 30 seconds, 50° C. for 30 seconds, and 68° C. for 3 minutes.

The contents from the PCR reaction were loaded onto a 1.2% agarose gel and electrophoresed. The DNA band of expected size (1.2 kb) was excised from the gel, placed on a GenElute Agarose spin column (Supelco), and spun for 10 minutes at maximum speed in a microfuge. The eluted DNA was ethanol-precipitated and resuspended in 6 μl H₂O for ligation.

H.4 Subcloning of Coding Region of CON222 via PCR

After a cDNA containing the full length CON222 open reading frame was obtained, the coding region of CON222 was then subcloned into a more useful vector as follows.

The purified PCR fragment described above, containing the CON222 coding sequence, was ligated into a commercial vector using Invitrogen's Original TA Cloning Kit. The ligation reaction was carried out as described above for CON193 in Example 1A.3. The resulting plasmid DNA from the culture was isolated using a Concert Rapid Plasmid Miniprep System (GibcoBRL) and sequenced to confirm that the plasmid contained the CON222 insert.

The subcloned insert in pCR2.1 was sequenced using the ABI PRISM™ 310 Genetic Analyzer (PE Applied Biosystems) which uses advanced capillary technology and the ABI PRISM™ BigDye™ Terminator Cycle Sequencing Ready Reaction Kit. Each cycle-sequence reaction contained 6 ml of H₂O, 8 ml of BigDye™ Terminator mix, 5 ml mini-prep DNA (0.1 mg/ml), and 1 ml of primer (25 ng/ml) and was performed in a Perkin-Elmer 9600 thermocycler with 25 cycles of 96° C. for 10 seconds, 50° C. for 10 seconds, and 60° C. for 4 minutes. The product was purified using a Centriflex™ gel filtration cartridge, vacuum dried and dissolved in 16 ml of Template Suppression Reagent (PE Applied Biosystems). The samples were heated at 95° C. for 5 minutes then placed in the 310 Genetic Analyzer.

Upon confirmation of the insert, the same transformant was used to inoculate a 50 ml culture of LB medium. The culture was grown for 16 hours at 37° C., and centrifuged into a cell pellet. Plasmid DNA was purified from the pellet using a Qiagen Plasmid Midi Kit and again sequenced to confirm successful cloning of the CON222 insert, as described above.

1. Cloning of CON215 G Protein-Coupled Receptor

1.1 Database Search Results

The database searching identified Clone 1452259H1 in the Incyte database as an interesting candidate sequence. The sequence from 1452259H1 clone was used to search the Incyte fill-length database and matched the entry 1650519CB1. An inspection of the clones that made up 1650519CB1 indicated that Incyte Clone 2796157H1 probably contained the full-length coding region. Sequence analysis of Incyte Clone 2796157H1 indicated that it contains the entire coding region for a previously unidentified GPCR, which was designated “CON215”, along with 12 nucleotides of 5′ untranslated region, 63 nucleotides of 3′ untranslated region and a poly A⁺ tail. The DNA and deduced amino acid sequences for CON215 are set forth in SEQ ID NOS: 17 and 18, respectively. A database search with this CON215 sequence showed a 47% match to the human probable G protein-coupled receptor KIA0001.

Since the untranslated regions were relatively short, it was not necessary to remove the coding region of CON215 from the pINCY vector (Incyte) and the construct is referred to as pINCY-CON215. The Incyte Clone 2796157H1 was sequenced using the ABI PRISM™ 310 Genetic Analyzer (PE Applied Biosystems) which uses advanced capillary electrophoresis technology and the ABI PRISM™ BigDye™ Terminator Cycle Sequencing Ready Reaction Kit as described above for CON222 in Example 1H.4.

J. Cloning of CON217 G Protein-Coupled Receptor

J.1 Database Search Results

The Incyte database search identified EST 3700658H1 as an interesting candidate sequence. The EST sequence No. 3700658H1 was used to search the Incyte full length database. This search identified Incyte clone No. 3356166H1 as a clone that potentially contained a full length GPCR corresponding to the selected EST.

The 3356166H1 clone was obtained from Incyte and sequenced using an ABI377 fluorescence-based sequencer (and the ABI PRISM™ Ready Dye-Deoxy Terminator kit with Taq FS™ polymerase as described above for CON193 in Example 1A.1.

Sequencing of Incyte Clone No. 3356166H1 revealed a 2480 basepair sequence as shown in SEQ NO: 19. Using a FORTRAN computer program called “tmtrest.all” [Parodi et al., Comput. Appl. Biosci., 5: 527-535 (1994)], Clone No. 3356166H1 was deduced to contain seven transmembrane-spanning domains (TMI-TMVII) and was designated as “CON217” (SEQ ID NO: 20). The following experiments were performed to subclone and isolate the full length coding sequence of CON217 from Incyte Clone No. 3356166H1.

J.2 Subcloning of the Coding Region of GPCR217

To subclone the full length coding sequence of CON217, PCR primers were designed based on the 5′ and 3′ ends of the open reading frame that was identified in the Incyte Clone No. 3356166H1. The first primer, designated as LW1448, has the sequence 5′AAGCGGTACCATGTTAGCCAACAGCTCCTC3′ (SEQ ID NO: 66) which corresponds to nucleotides 42 to 62 of SEQ ID NO: 19 (underlined). The second primer, designated LW1449, has the sequence 5′AAGCTCTAGATCAGAGGGCGGAATCCTGG3′ (SEQ ID NO: 67) which is the reverse complement of nucleotides 1142 to 1160 of SEQ ID NO: 20 (underlined). The primers also include recognition sequences (bold) for the restriction enzymes KpnI and XbaI, respectively.

PCR was performed in a 50 ml reaction containing 32.5 ml of H₂O, 5 ml of 10× Pfx Amplification buffer (GibcoBRL), 5 ml of 10× PCR Enhancer solution (GibcoBRL), 1.5 ml of 50 mM MgSO₄, 2 ml of 10 mM dNTP's (dATP, dCTP, dGTP, dTTP), 3 ml 3356166H1 mini-prep DNA (0.125 mg/ml obtained with the Concert Rapid Plasmid Miniprep System; GibcoBRL), 0.3 ml of Primer LW1448 (1 mg/ml) (SEQ ID NO: 3), 0.3 ml of Primer LW1449 (1 mg/ml) (SEQ ID NO: 4), 0.5 ml Platinum Pfx DNA polymerase (2.5 U/ml; GibcoBRL). The PCR reaction was started with 1 cycle of 94° C. for 2 minutes followed by 25 cycles at 94° C. for 30 seconds, 55° C. for 30 seconds, 68° C. for 1.3 minutes.

The contents from the PCR reaction were loaded onto a 1.2% agarose gel and electrophoresed. The DNA band of expected size (˜1.1 kb) was excised from the gel, placed on a GenElute Agarose spin column (Supelco), and spun for 10 minutes at maximum speed in a microfuge. The eluted DNA was ethanol-precipitated and resuspended in 6 μl of H₂O for ligation.

The purified PCR fragment, containing the CON217 coding sequence, was ligated into a commercial vector designated pCR2.1 using Invitrogen's Original TA Cloning Kit. The ligation reaction was carried out as described above for CON193 in Example 1A.3. The resulting plasmid DNA from the culture was isolated using a Concert Rapid Plasmid Miniprep System (GibcoBRL) and sequenced to confirm that the plasmid contained the CON217 insert and to confirm that no errors were introduced during PCR amplification. The resulting construct was denoted as pCR-CON217.

The final subclone was sequenced using the ABI PRISM™ 310 Genetic Analyzer (PE Applied Biosystems) which uses advanced capillary electrophoresis technology and the ABI PRISM™ Terminator Cycle Sequencing Ready Reaction Kit as described above for CON222 in Example 1H.4.

### EXAMPLE 2

### Analysis of G Protein-Coupled Receptor Sequence

A. CON193

The DNA and deduced amino acid sequence for CON193 are set forth in SEQ ID NOS: 1 and 2, respectively. Beginning with the initiation codon (methionine), the CON193 genomic Clone contains an open reading frame of 963 nucleotides encoding 321 amino acids, followed by a stop codon. Using a FORTRAN computer program called “tmtrest.all” [Parodi et al., Comput. Appl. Biosci., 5: 527-535 (1994)], CON193 was shown to contain seven transmembrane-spanning domains corresponding to residues 30-49 (1TM), 61-81 (2TM), 103-122 (3TM), 146-165 (4TM), 199-222 (5TM), 243-262 (6TM), and 270-295 (7TM) of SEQ ID NO: 2. These transmembrane domains define first (“N-terminal,” residues 1-29), second (“first EC loop,” residues 82-102), third (“second EC loop,” residues 166-198), and fourth (“third EC loop,” residues 263-269) extracellular domains, as well as first (“first IC loop,” residues 50-60), second (“second IC loop,” residues 123-145), third (“third IC loop.” residues 223-242), and fourth (“C-terminal,” residues 296-321) intracellular domains.

Inspection of the CON193 amino acid sequence (SEQ ID NO: 2) reveals that this GPCR contains a DRY sequence following the third transmembrane domain (3TM) and a PIVY sequence found in the sixth transmembrane domain (TM6). In addition, the CON193 polynucleotide sequence was compared to sequences of known genes. CON193 is 45% identical and 72% similar to the mouse olfactory receptor gene S19 [see Malnic et al., Cell 96:713-723 (1999)]. This level of sequence similarity suggests that CON193 is a novel GPCR.

The CON193 cDNA clone (SEQ ID NO:1) was deposited with the National Center for Agricultural Utilization Research at the United States Department of Agriculture 1815 North University Street, Peoria, Ill. 61604 in accordance with the Budapest Treaty on Jan. 18, 2000. The clone was given accession no. B-30250.

B. CON166

The DNA and deduced amino acid sequence for CON166 are set forth in SEQ ID NOS: 3 and 4, respectively. Beginning with the initiation codon (methionine), the CON166 genomic clone contains an open reading frame of 1,011 nucleotides encoding 337 amino acids, followed by a stop codon. Using a FORTRAN computer program called “tmtrest.all” [Parodi et al., Comput. Appl. Biosci., 5: 527-535 (1994)], CON166 was shown to contain seven transmembrane-spanning domains corresponding to the following residues presented in SEQ ID NO: 4: 1TM (30-49), 2TM (59-79), 3TM (99-119), 4TM (141-161), 5TM (191-215), 6TM (231-251), and 7TM (277-296). These transmembrane domains define first (“N-terminal,” residues 1-29), second (“first EC loop,” residues 80-98), third (“second EC loop,” residues 162-190), and fourth (“third EC loop,” residues 252-276), extracellular domains as well as first (“first IC loop,” residues 50-58), second (“second IC loop,” residues 120-140), third (“third IC loop,” residues 216-230), and fourth (“C-terminal,” residues 297-337) intracellular domains.

Inspection of the CON166 amino acid sequence (SEQ ID NO:2) reveals that this GPCR contains an FRC sequence following the third transmembrane domain (3TM), which is typically occupied by a consensus DRY sequence in other GPCRs; a PLLY sequence is also found in the seventh transmembrane domain (7TM). In addition, the CON166 polynucleotide sequence was compared to sequences of known genes. CON166 is 44% identical and 62% similar to a T-cell-specific G protein-coupled receptor of Gallus gallus found in the TREMBL database (Accession No. L06109). This level of sequence similarity suggests that CON166 is a novel GPCR.

The CON166 cDNA clone (SEQ ID NO:3) was deposited with the National Center for Agricultural Utilization Research at the United States Department of Agriculture 1815 North University Street, Peoria, Ill. 61604 in accordance with the Budapest Treaty on Jan. 18, 2000. The clone was given accession no. B-30248.

C. CON103

The DNA and deduced amino acid sequence for CON103 are set forth in SEQ ID NOS: 5 and 6, respectively. Beginning with the initiation codon (methionine), the CON103 genomic clone contains an open reading frame of 1,152 nucleotides encoding 384 amino acids, followed by a stop codon and a short open reading frame (SEQ ID NO: 5). Using a FORTRAN computer program called “tmtrest.all” [Parodi et al., Comput. Appl. Biosci., 5: 527-535 (1994)], CON103 was shown to contain seven transmembrane-spanning domains corresponding to the following residues in SEQ ID NO: 6: 54-77 (1TM), 89-108 (2TM), 134-149 (3TM), 167-188 (4TM), 216-240 (5TM), 258-283 (6TM), and 301-320 (7TM). These transmembrane domains define first (“N-terminal,” residues 1-53), second (“first EC loop,” residues 109-133), third (“second EC loop,” residues 189-215), and fourth (“third EC loop,” residues 284-300) extracellular domains, as well as first (“first IC loop,” residues 78-88), second (“second IC loop,” residues 150-166), third (“third IC loop,” residues 241-257), and fourth (“C-terminal,” residues 321-384) intracellular domains.

Inspection of the CON103 amino acid sequence (SEQ ID NO: 6) reveals that this GPCR contains an NRY sequence following the third transmembrane domain (3TM), which is typically occupied by a consensus DRY sequence in other GPCRs. In addition, the CON103 polynucleotide sequence was compared to sequences of known genes. CON103 is 36% identical to GPR31 (GenBank Accession No. U65402) and 31% identical to the P2Y1 purinergic receptor (GenBank Accession No. S81950). This level of sequence similarity indicates that CON103 is a novel GPCR.

The CON103 cDNA clone (SEQ ID NO:5) was deposited with the National Center for Agricultural Utilization Research at the United States Department of Agriculture 1815 North University Street, Peoria, Ill. 61604 in accordance with the Budapest Treaty on Jan. 18, 2000. The clone was given accession no. B-30247.

D. CON203

The DNA and deduced amino acid sequence for CON203 are set forth in SEQ ID NOS: 7 and 8, respectively. Beginning with the initiation codon (methionine), the CON203 genomic clone contains an open reading frame of 999 nucleotides encoding 333 amino acids, followed by a stop codon. Using a FORTRAN computer program called “tmtrest.all” [Parodi et al., Comput. Appl. Biosci., 5: 527-535 (1994)], CON203 was shown to contain seven transmembrane-spanning domains corresponding to the following residues of SEQ ID NO: 7: nucleotides 29-53 (1TM), 63-82 (2TM), 97-118 (3TM), 136-160 (4TM), 189-211 (5TM), 232-252 (6TM), and 281-300 (7TM). These transmembrane domains define first (“N-terminal,” residues 1-28), second (“first EC loop,” residues 83-96), third (“second EC loop,” residues 161-188), and fourth (“third EC loop,” residues 253-280) extracellular domains, as well as first (“first IC loop,” residues 54-62), second (“second IC loop,” residues 119-135), third (“third IC loop,” residues 212-231), and fourth (“C-terminal,” residues 301-333) intracellular domains.

Inspection of the CON203 amino acid sequence (SEQ ID NO: 8) reveals that this GPCR contains a DRF sequence following the third transmembrane domain (3TM), which is typically occupied by a consensus DRY sequence in other GPCRs; CON203 also exhibited a PLIY sequence in the seventh transmembrane domain (7TM). In addition, the CON203 polynucleotide sequence was compared to sequences of known genes. CON203 is 33% identical to a platelet activating receptor (GenBank Accession No. AF002986. This level of sequence similarity suggests that CON203 is a novel GPCR.

The CON203 cDNA clone (SEQ ID NO: 7) was deposited with the National Center for Agricultural Utilization Research at the United States Department of Agriculture 1815 North University Street, Peoria, Ill. 61604 in accordance with the Budapest Treaty on Jan. 18, 2000. The clone was given accession no. B-30254.

E. CON198

The DNA and deduced amino acid sequence for CON198 are set forth in SEQ ID NO: 9 and 10 respectively. Beginning with the initiator methionine, the CON198 genomic clone contains an open reading frame of 954 nucleotides encoding 318 amino acids, followed by a stop codon. It will be appreciated that residue 2 of SEQ ID NO: 10 also is a methionine. Amino-terminal sequencing of purified native or recombinant CON198 protein will provide an indication as to which methionine acts as an initiator codon in vivo. Using a FORTRAN computer program called “tmtrest.all” [Parodi et al., Comput. Appl. Biosci., 5: 527-535 (1994)], CON198 was deduced to contain seven transmembrane-spanning domains corresponding to residues 28-52 (TM1), 61-80 (TM2), 104-123 (TM3), 147-167 (TM4), 200-226 (TM5), 239-263 (TM6), and 274-295 (TM7) of SEQ ID NO: 10. These transmembrane domains define first (“N-terminal,” residues 1-27 or 2-27), second (“first EC loop,” residues 81-103), third (“second EC loop,” residues 168-199), and fourth (“third EC loop,” residues 264-273) extracellular domains as well as first (“first IC loop,” residues 53-60), second (“second IC loop,” residues 124-146), third (“third IC loop,” residues 227-238), and fourth (“C-terminal,” residues 296-318) intracellular domains.

CON198 contains a DRY sequence following the third transmembrane domain (TM3), a feature that is conserved in most GPCR. The most similar sequence in a public database. at the time of initial screening, was that of rat GPCR RA1c, which shared only 61% identity at the amino acid level.

The CON198 cDNA clone (SEQ ID NO: 9) was deposited with the National Center for Agricultural Utilization Research at the United States Department of Agriculture 1815 North University Street, Peoria, Ill. 61604 in accordance with the Budapest Treaty on Jan. 18, 2000. The clone was given accession no. B-30252.

F. CON197

The DNA and deduced amino acid sequence for CON197 are set forth in SEQ ID NO: 11 and 12, respectively. Beginning with the initiator methionine, the CON197 genomic clone contains an open reading frame of 921 nucleotides encoding 307 amino acids, followed by a stop codon. Using a FORTRAN computer program called “tmtrest.all” [Parodi et al., Comput. Appl. Biosci., 5: 527-535 (1994)], CON197 was deduced to contain seven transmembrane-spanning domains corresponding to residues 23-47 (TM1), 58-78 (TM2), 99-120 (TM3), 142-164 (TM4), 195-219 (TM5), 237-258 (TM6), and 270-289 (TM7) of SEQ ID NO: 12. These transmembrane domains define first (“N-terminal” residues 1-22), second (“first EC loop” residues 79-98), third (“second EC loop” residues 165-194), and fourth (“third EC loop” residues 259-269) extracellular domains as well as first (“first IC loop” residues 48-57), second (“second IC loop” residues 121-141), third (“third IC loop” residues 220-236), and fourth (“C-terminal” residues 290-309) intracellular domains.

CON197 contains a DRY sequence following the third transmembrane domain (TM3), a feature that is conserved in most GPCR. The most similar sequence in a public database, at the time of initial screening, was that of an olfactory receptor, which shared only 42% identity at the amino acid level.

The CON197 cDNA clone (SEQ ID NO: 11) was deposited with the National Center for Agricultural Utilization Research at the United States Department of Agriculture 1815 North University Street, Peoria, Ill. 61604 in accordance with the Budapest Treaty on Jan. 18, 2000. The clone was given accession no. B-30251.

G. CON202

The DNA and deduced amino acid sequence for this phage insert, termed “CON202”, are set forth in SEQ ID NO: 13 and 14, respectively. The CON202 open reading frame, as depicted in SEQ ID NO: 14, begins with the initiator methionine and spans 1110 nucleotides which encode 370 amino acids, followed by a stop codon. Since this gene was isolated from genomic DNA and there are no apparent interruptions in the sequence, it is likely that CON202 contains no introns within the coding region. The full length clone of CON202 contained seven transmembrane-spanning domains corresponding to residues, 24 to 46 (TM1) 57 to 77 (TM2), 96 to 117 (TM3), 135 to 159, (TM4) TMV comprises 184 to 202 (TM5), 286 to 308 (TM6), 316 to 339 (TM7) of SEQ ID NO: 14. TM2 terminates with PFVC instead of the characteristic PXXY. TM3 is followed by the sequence TRY instead of the characteristic DRY. These transmembrane domains define first (“N-terminal,” residues 1-23), second (“first EC loop,” residues 78-95), third (“second EC loop,” residues 160-183), and fourth (“third EC loop,” residues 309-315) extracellular domains as well as first (“first IC loop,” residues 47-56), second (“second IC loop,” residues 118-134), third (“third IC loop,” residues 203-285), and fourth (“C-terminal,” residues 340-370) intracellular domains.

The CON202 cDNA clone (SEQ ID NO: 13) was deposited with the National Center for Agricultural Utilization Research at the United States Department of Agriculture 1815 North University Street, Peoria, Ill. 61604 in accordance with the Budapest Treaty on Jan. 18, 2000. The clone was given accession no. B-30253.

H. CON222

The sequence of CON222 coding region deduced the DNA and amino acid sequence set forth in SEQ ID NO: 15 and 16, respectively. The open reading frame that is depicted in SEQ ID NO: 16 begins with an initiator codon and spans 1188 nucleotides which encode 396 amino acids, followed by a stop codon.

The full length clone of CON222 contains seven transmembrane-spanning domains corresponding to residues 42-65 (TM 1) 79-103, (TM2), 125-156, (TM3), 167-188 (TM4), 217-241 (TM5). 268-290 (TM6), 301-320 (TM7) of SEQ ID NO: 16. TM2 is followed by a FRC sequence and TM7 contains a PILY sequence within. These transmembrane domains define first (“N-terminal,” residues 1-41). second (“first EC loop,” residues 104-124), third (“second EC loop,” residues 189-216), and fourth (“third EC loop.” residues 291-300) extracellular domains as well as first (“first IC loop,” residues 66-78). second (“second IC loop,” residues 157-166), third (“third IC loop,” residues 242-267), and fourth (“C-terminal,” residues 321-396) intracellular domains. A search of the public database indicated that CON222 is about 35% identical to a unique GPCR found in the nervous system of Lymnaea stagnalis.

The CON222 cDNA clone (SEQ ID NO: 15) was deposited with the National Center for Agricultural Utilization Research at the United States Department of Agriculture 1815 North University Street, Peoria, Ill. 61604 in accordance with the Budapest Treaty on Jan. 18, 2000. The clone was given accession no. B-30257.

I. CON215

The DNA and deduced amino acid sequence for CON215 are set forth in SEQ ID NO: 17 and 18, respectively. Beginning with the initiator methionine, the CON215 genomic clone contains an open reading frame of 1074 nucleotides encoding 358 amino acids, followed by a stop codon. Using a FORTRAN computer program called “tmtrest.all” [Parodi et al., Comput. Appl. Biosci., 5: 527-535 (1994)], CON215 was deduced to contain seven transmembrane-spanning domains corresponding to residues 42-66 (TM 1), 81-99 (TM2), 116-137 (TM3), 156-180 (TM4), 210-234 (TM5), 256-275 (TM6), and 308-328 (TM7) of SEQ ID NO: 18. These transmembrane domains define first (“N-terminal,” residues 1-41), second (“first EC loop,” residues 100-115), third (“second EC loop,” residues 181-209), and fourth (“third EC loop,” residues 276-307) extracellular domains as well as first (“first IC loop,” residues 67-80), second (“second IC loop,” residues 138-155), third (“third IC loop,” residues 235-255), and fourth (“C-terminal,” residues 329-358) intracellular domains.

CON215 contains a DRY sequence following the third transmembrane domain (TM3), a feature that is conserved in most GPCR. CON215 also contains a PIIY sequence within the seventh transmembrane domain (TM7).

The CON215 cDNA clone (SEQ ID NO: 17) was deposited with the National Center for Agricultural Utilization Research at the United States Department of Agriculture 1815 North University Street, Peoria, Ill. 61604 in accordance with the Budapest Treaty on Jan. 18, 2000. The clone was given accession no. B-30255.

J. CON217

The DNA and deduced amino acid sequences of CON217 are set forth as SEQ ID NO: 19 and 20, respectively. The open reading frame that is depicted in SEQ ID NO: 2 begins with an initiator methionine codon and spans 1116 nucleotides which encode 372 amino acids, followed by a stop codon. In addition, the nucleotide sequence consists of 41 bp in the 5′ untranslated region and 1323 bp in the 3′ untranslated region.

The full length clone of CON217 contains seven transmembrane-spanning domains as indicated by the FORTRAN computer program “tmtrest.all” [Parodi et al., Comput. Appl. Biosci., 5: 527-535 (1994)] which corresponds to 29-50 (TM1), 57-75 (TM2), 96-117 (TM3), 137-160 (TM4), 188-210 (TM5), 235-258 (TM6), 277-297 (TM7). TM3 is followed by a DRY sequence and TM7 contains a PLVY sequence within. These transmembrane domains define first (“N-terminal,” residues 1-28), second (“first EC loop,” residues 76-95), third (“second EC loop,” residues 161-187), and fourth (“third EC loop,” residues 259-276) extracellular domains as well as first (“first IC loop,” residues 51-56), second (“second IC loop,” residues 118-136), third (“third IC loop,” residues 211-234), and fourth (“C-terminal,” residues 298-372) intracellular domains. A search of the public database indicated that CON217 is about 41% identical to GPR23 (Genebank Accession No.: U66578) and to a purinergic receptor P2Y9 (Genebank Accession No.: U90322).

The CON215 cDNA clone (SEQ ID NO: 19) was deposited with the National Center for Agricultural Utilization Research at the United States Department of Agriculture 1815 North University Street, Peoria, Ill. 61604 in accordance with the Budapest Treaty on Jan. 18, 2000. The clone was given accession no. B-30256.

K. Summary of Deposits

The polynucleotides (SEQ ID NO: 1, 3, 5, 7, 9, 11, 13, 15 and 17) encoding the GPCR polypeptides of the invention were deposited with the Agricultural Research Service Culture Collection (NRRL) at the National Center Agricultural Utilization Research at the U.S. Department of the Agriculture 1815 North University Street, Peoria, Ill. 61604. These deposits were made in accordance with the Budapest Treaty on the International Recognition of the Deposit of Microorganism for the Purposes of Patent Procedures. The table below lists the details of these deposits.

| GPCR | SEQ ID NO: | NRRL No. | Deposit Date |
|---|---|---|---|
| CON193 | 1 | B-30250 | Jan. 18, 2000 |
| CON166 | 3 | B-30248 | Jan. 18, 2000 |
| CON103 | 5 | B-30247 | Jan. 18, 2000 |
| CON203 | 7 | B-30254 | Jan. 18, 2000 |
| CON198 | 9 | B-30252 | Jan. 18, 2000 |
| CON197 | 11 | B-30251 | Jan. 18, 2000 |
| CON202 | 13 | B-30253 | Jan. 18, 2000 |
| CON222 | 15 | B-30257 | Jan. 18, 2000 |
| CON215 | 17 | B-30255 | Jan. 18, 2000 |
| CON217 | 19 | B-30256 | Jan. 18, 2000 |

### EXAMPLE 3

### Hybridization Analysis Demonstrates that the GPCRs are Expressed in the Brain

The expression of GPCR polynucloetides in mammals, such as the rat, was investigated by in situ hybridization histochemistry. Coronal and sagittal rat brain cryosections (20 μm thick) were prepared using a Reichert-Jung cryostat. Individual sections were thaw-mounted onto silanized, nuclease-free slides (CEL Associates. Inc., Houston, Tex.), and stored at −80° C. Sections were processed starting with post-fixation in cold 4% paraformaldehyde, rinsed in cold phosphate-buffered saline (PBS), acetylated using acetic anhydride in triethanolamine buffer, and dehydrated through a series of alcohol washes in 70%, 95%, and 100% alcohol at room temperature. Subsequently, sections were delipidated in chloroform, followed by rehydration through successive exposure to 100% and 95% alcohol at room temperature. Microscope slides containing processed cryosections were allowed to air dry prior to hybridization.

A. CON193

A CON193-specific probe was generated using PCR. The probe consisted of a 270 bp fragment containing sequence at the 3′ end of CON-193. The primers for PCR amplification were LW1248 [5′-GCATGAATTCCAATATACTTCCCCATACCTAC-3′; SEQ ID NO: 26) and LW 1249 [5′-GCATGGATCCGGAAAAGAAGGAGAAGAAAG-3′; SEQ ID NO: 27), which introduced terminal EcoRI and BamHI restriction sites into the PCR product. Following PCR amplification, the fragment was digested with EcoRI and BamHI and cloned into pBluescriptII cleaved with the same enzymes. For production of a probe specific for the sense strand of CON193, the CON193 Clone in pBluescriptII was linearized with BamHI, which provided a substrate for labeled run-off transcripts (i.e., cRNA riboprobes) using the vector-borne T7 promoter and commercially available T7 RNA polymerase. A probe specific for the antisense strand of CON193 was Also readily prepared using the CON193 Clone in pBluescriptII by cleaving the recombinant plasmid with EcoRI to generate a linearized substrate for the production of labeled run-off cRNA transcripts using the T3 promoter and cognate polymerase. The riboprobes were labeled with [³⁵S]-UTP to yield a specific activity of 0.81×10⁶ cpm/pmol for antisense riboprobes and 0.55×10⁶ cpm/pmol for sense-strand riboprobes. Both riboprobes were subsequently denatured by incubating at 70° C. for 3 minutes and added (2 pmol/ml) to hybridization buffer which contained 50% formamide, 10% dextran, 0.3 M NaCl, 10 mM Tris (pH 8.0), 1 mM EDTA. 1× Denhardt's Solution, and 10 mM dithiothreitol. Microscope slides containing sequential brain cryosections were independently exposed to 45 μl of hybridization solution per slide and silanized cover slips were placed over the sections being exposed to hybridization solution. Sections were incubated overnight (15-18 hours) at 52° C. to allow hybridization to occur. Equivalent series of cryosections were exposed to sense or antisense CON193-specific cRNA riboprobes.

Following the hybridization period, coverslips were washed off the slides in 1×SSC. Slides were subjected to RNase A treatment by incubation in a buffer containing 20 μg/ml RNase A, 10 mM Tris (pH 8.0), 0.5 M NaCl and 1 mM EDTA for 45 minutes at 37° C. The cryosections were then subjected to three high-stringency washes in 0.1×SSC at 52° C. for 20 minutes each. Following the series of washes, cryosections were dehydrated by consecutive exposure to 70%, 95%, and 100% ammonium acetate in alcohol, followed by air drying and exposure to Kodak BioMax MR-1 film. After 13 days of exposure, the film was developed. Based on these results, brain sections that gave rise to positive hybridization signals were coated with Kodak NTB-2 nuclear track emulsion and the slides were stored in the dark for 32 days The slides were then developed and counterstained with hematoxylin. Emulsion-coated sections were analyzed microscopically to determine the specificity of labeling. The signal was determined to be specific if autoradiographic grains (generated by antisense probe hybridization) were clearly associated with crystal violet-stained cell bodies. Autoradiographic grains found between cell bodies indicates non-specific binding.

Specific labeling with the antisense probe occurred at low levels in the cortex and in the substantia nigra-pars compacta (SN-c). The specificity of labeling was confirmed by microscopic analysis of emulsion-coated cryosections, as described above. In contrast, hybridization using the riboprobe specific for the sense strand of CON193 did not result in specific tissue labeling. The observed regional distribution of CON193 mRNA suggests that ligands for this GPCR may be involved in signal transductions important for cellular processes underlying neurological functioning. In addition, expression of CON193 in the brain provides an indication that modulators of CON193 activity have utility for treating neurological disorders, including but not limited to, schizophrenia, depression, anxiety, bipolar disease, epilepsy, neuritis, neurasthenia, neuropathy, neuroses, and the like. Use of CON193 modulators, including CON193 ligands and anti-CON193 antibodies, to treat individuals having such disease states is intended as an aspect of the invention.

B. CON166

A CON166-specific probe was generated using PCR as described above for CON193 in Example 3A (but using CON166-specific primers). The probe consisted of a 259 bp fragment containing sequence at the 3′ end of CON-166 (nucleotides 715-974 of SEQ ID NO: 1) and containing terminal EcoRI and BamHI restriction sites. The riboprobes were labeled with [³⁵S]-UTP to yield a specific activity of 0.40×10⁶ cpm/pmol for antisense riboprobes and 0.65×10⁶ cpm/pmol for sense-strand riboprobes Hybridization with the riboprobes and subsequent washing of the slides was carried out as described above for CON193 in Example 3A.

Specific labeling with the antisense probe occurred in cortical regions, including the piriform complex, neostriatum, thalamus and hippocampus. The specificity of labeling was confirmed by microscopic analysis of emulsion-coated cryosections. These sections revealed that the autoradiographic grains resulting from antisense riboprobe in situ hybridizations were distributed over cell bodies rather than trapped between cell bodies. In contrast, hybridization using the riboprobe specific for the sense strand of CON166 produced a faint signal in the hippocampus only, but even this signal was found to be non-specific upon microscopic examination. The observed regional distribution of CON166 mRNA suggests that ligands for this GPCR may be involved in signal transductions important for cellular processes underlying neurological functioning. In addition, expression of CON166 in the brain provides an indication that modulators of CON166 activity have utility for treating neurological disorders, including but not limited to, schizophrenia, affective disorders, ADHD/ADD (i.e., Attention Deficit-Hyperactivity Disorder/Attention Deficit Disorder), and neural disorders such as Alzheimer's disease, Parkinson's disease, migraine, and senile dementia. Some other diseases for which modulators of CON166 may have utility include depression, anxiety bipolar disease, epilepsy, neuritis. neurasthenia, neuropathy, neuroses, and the like. Use of CON166 modulators, including CON166 ligands and anti-CON166 antibodies, to treat individuals having such disease states is intended as an aspect of the invention.

C. CON103

A cocktail of two CON103-specific antisense oligonucleotide probes (CON103a and CON103b) were used because of the relatively high GC content of the CON103 coding region. The CON103a sequence (5′TTTATTAATATTGGAAGGGACAAACTGGAGAGCACAGAACAT3′; SEQ ID NO: 72) corresponds to the reverse complement of nucleotides 2196-2237 of SEQ ID NO: 5 and CON103b sequence (5′AAAGCCACCATGGAAGCCATGCCAAAGATGATGCTGGGCAAGAA3′; SEQ ID NO: 73) corresponds to the reverse complement of nucleotides 195-1538 of SEQ ID NO: 5. Terminal deoxynucleotidyltransferase and [α-³³P]dATP were used to 3′ end-label CON103a (1.36×107 cpm/pmol) and CON103b (9.1×10⁶ cpm/pmol). The probes were denatured by incubation at 70° C. for three minutes and added to hybridization buffer containing 50% formamide, 10% dextran, 0.3 M NaCl, 10 mM Tris (pH 8.0), 1 mM EDTA, 1× Denhardt's Solution, and 200 mM dithiothreitol. The final concentration of each radiolabeled probe was 2 pmol/ml of hybridization solution. Microscope slides containing sequential brain cryosections were independently exposed to 45 μl of hybridization solution (containing the antisense oligonucleotide probes CON103a and CON103b) per slide and silanized cover slips were placed over the sections being exposed to hybridization solution. Sections were incubated overnight (15-18 hours) at 37° C. to allow hybridization to occur.

Following the hybridization period, coverslips were washed off the slides in 1×SSC. The cryosections were then subjected to three high-stringency washes in 1×SSC at 65° C. for 20 minutes each. Following two room-temperature washes, cryosections were dehydrated by consecutive exposure to 70%, 95%, and 100% ethanol (0.3 M ammonium acetate added to 70% and 95% ethanol solutions), followed by air drying and exposure to Kodak BioMax MR-1 film. After 28 days of exposure, the film was developed. Based on these results, brain sections that showed positive hybridization signals were coated with Kodak NTB-2 nuclear track emulsion and the slides were stored in the dark for four months. The slides were then developed and counterstained with hematoxylin. Emulsion-coated sections were analyzed microscopically to determine the specificity of labeling. The signal was determined to be specific if autoradiographic grains (generated by antisense probe hybridization) were present over cell bodies and not trapped between cell bodies.

Specific labeling with the antisense probe occurred in all cortical regions, including the piriform cortex and hippocampus. The specificity of labeling was confirmed by microscopic analysis of emulsion-coated cryosections. These sections revealed that the autoradiographic grains resulting from antisense riboprobe in situ hybridizations were distributed over cell bodies rather than trapped between cell bodies. The observed distribution of CON103 mRNA in the cortical and paralimbic regions of the mammalian brain suggests that ligands for this GPCR may be involved in signal transductions important for cellular processes underlying neurological functioning. In addition, expression of CON103 in the brain provides an indication that modulators of CON103 activity have utility for treating neurological and neuropsychiatric disorders, including but not limited to, schizophrenia, depression, anxiety, attention deficit disorder (with or without hyperactivity), bipolar disease, epilepsy, migraine, neuritis, neurasthenia, neuropathy, neuroses, obesity, Parkinson's disease, other dementias, and the like. Use of CON103 modulators, including CON103 ligands and anti-CON103 antibodies, to treat individuals having such disease states is intended as an aspect of the invention.

D. CON203

CON203-specific cRNA probes were prepared using conventional techniques. Initially, a 293 bp fragment of the CON203 coding region, with a BamHI site and an EcoRI site disposed on opposite ends, was prepared by PCR using primers LW1314 (5′-GCATGAATTCCCACCTTCATCATCTACCTC-3′; SEQ ID NO: 40) and LW1315 (5′-GCATGGATCCGAAGACCAAAAAGACCCAG-3′; SEQ ID NO: 41). LW1314 includes an EcoRI site and additional protective residues at its 5′ terminus, with the rest of the sequence corresponding to CON203 coding nucleotides 164-183, which correspond to positions 309-328 of SEQ ID NO: 7. LW1135 includes 5′ protective nucleotides and a BamHI site, with the rest of the sequence corresponding to the complement of CON203 coding nucleotides 438-456, which correspond to positions 583-601 of SEQ ID NO: 7. The PCR-amplified fragment was then digested with BamHI and EcoRI and ligated into the corresponding sites of pBluescript II to yield pCon203 BS. The recombinant clone was then linearized either with BamHI or EcoRI. Linearization with BamHI provided a substrate for in vitro expression of a sense-strand cRNA probe using the vector-borne T7 promoter. Digestion with EcoRI was used to provide a substrate for in vitro transcription using the vector-borne T3 promoter to generate an anti-sense cRNA probe. In vitro transcriptions were performed in the presence of [³⁵S] UTP, thereby yielding sense- and anti-sense strand riboprobes having specific radioactivities of 5.38×10⁷ cpm/pmol and 5.34×10⁷ cpm/pmol, respectively. Hybridization with the riboprobes and subsequent washing of the slides was carried out as described above for CON193 in Example 3A. Subsequently, the slides were exposed to Kodak BioMax MR-1 film. After 9 days of exposure, the film was developed. Based on these results, brain sections that gave rise to positive hybridization signals were coated with Kodak NTB-2 nuclear track emulsion and the slides were stored in the dark for 25 days. The slides were then developed as described above for CON193 in Example 3A.

Specific labeling with the antisense probe occurred in several limbic and paralimbic regions, as well as areas thought to be involved in voluntary motor control. In particular, the probe hybridized to CON203 mRNAs in the following regions of the brain: cortical regions, including the piriform cortex, neostriatum, lateral olfactory tract, hypothalamic nuclei, bed nucleus of the stria terminals, amygdala, hippocampus, reticular thalamus and other thalamic regions, subthalamic nucleus, and the red nucleus. The specificity of labeling was confirmed by microscopic analysis of emulsion-coated cryosections. These sections revealed that the autoradiographic grains resulting from antisense riboprobe in situ hybridizations were distributed over cell bodies rather than trapped between cell bodies. Confirming expression of CON203 mRNA, the sense-strand riboprobe did not show specific hybridization. The observed distribution of CON203 mRNA in the cortical (particularly, motor circuits) and paralimbic regions of the mammalian brain suggests that CON203 and the ligands for this GPCR may be involved in signal transductions important for cellular processes underlying neurological functioning. In addition, expression of CON203 in the brain provides an indication that modulators of CON203 activity have utility for treating neurological disorders, including but not limited to, schizophrenia, depression, anxiety, bipolar disease, epilepsy, migraine, attention deficit disorder (with or without hyperactivity), neuritis, neurasthenia, neuropathy, neuroses, Parkinson's disease, dementia, obesity, and the like. Use of CON203 modulators, including CON203 ligands and anti-CON203 antibodies, to treat individuals having such disease states is intended as an aspect of the invention.

E. CON198

A 266 bp fragment of CON198 containing EcoRI and BamHI restriction sites was amplified from the full-length clone by PCR, using the primers LW1308: 5′-GCATGAATTCACTCACTTCTCATCTCCTTC-3′ (SEQ ID NO: 46) and LW1309:5′-GCATGGATCCAATCTCCTTTGTCTTCACTC-3′ (SEQ ID NO: 47) Primer LW1308 contains an EcoRI site (underlined) followed by sequence identical to nucleotides 638-657 of SEQ ID NO: 9. Primer LW1309 contain a BamHI site (underlined) followed by sequence complementary to nucleotides 903-884 of SEQ ID NO: 9. The amplification product was digested with EcoRI and BamHI, and then subcloned into an EcoRI- and BamHI-digested pBluescript II vector (Stratagene). The 266 amplified and subcloned basepairs correspond to nucleotides 638 to 903 of SEQ ID NO: 9.

The subcloned CON198-Bluescript construct was used to generate strand-specific probes for the in situ hybridization experiments. The construct was linearized with BamHI, for labeling with T7 polymerase (sense), or EtoRI, for T3 polymerase (antisense), and used as a template for in vitro transcription of sense and antisense cRNA riboprobes. The riboprobes were labeled with ³⁵S-UTP to yield a specific activity of 0.45×10⁶ cpm/pmol for antisense and 0.732×10⁶ cpm/pmol for sense probe. Hybridization with the riboprobes and subsequent washing of the slides was carried out as described above for CON193 in Example 3A.

Specific labeling with the antisense probe showed distribution of CON198 mRNA in the rat brain in several linibic and paralimbic regions as well as areas thought to be involved in voluntary motor control. Labelled regions included cortical regions, piriform cortex, hypothalamic nuclei (paraventricular nucleus, supraoptic nucleus, suprachiasmatic nucleus), hippocampus, reticular thalmus, substantia nigra-pars compacta (SN-C), ventral tegmental area, and the red nucleus. The specificity of labeling was confirmed by microscopic analysis of emulsion coated sections. These sections revealed that the autoradiographic grains generated by the antisense probe were distributed over cell bodies rather than trapped between cell bodies. Sense probe did not generate specific labeling.

The observed regional distribution of CON198 MRNA provides a therapeutic indication for natural ligands for CON198 as well as modulators of CON198 activity, such as anti-CON198 antibody substances or small molecules that agonize or antagonize ligand-mediated CON198 signalling. In particular, the expression pattern provides an indication that such molecules will have utility for treating neurological and/or psychiatric diseases, including but not limited to schizophrenia, depression, anxiety, bipolar disease, affective disorders, ADHD/ADD, epilepsy, neuritis, neurasthenia, neuropathy, neuroses, Alzheimer's disease, Parkinson's disease, migraine, senile dementia, and the like. Use of CON198 modulators, including CON198 ligands and anti-CON198 antibodies, to treat individuals having such disease states is intended as an aspect of the invention. Such modulators are administered by any means effective to safely deliver the modulators to the CON198-expressing cells, including but not limited to oral administration, inhalation, or injection of compositions comprising the modulators in a pharmaceutically acceptable diluent, adjuvant, or carrier. Efficacy of treatment can initially be determined in any accepted animal model that provides a biochemical or behavioral marker that correlates with disease severity or treatment efficacy.

F. CON197

A 261 bp fragment of CON197 containing EcoRI and BamHI restriction sites was amplified from the full-length clone by PCR, using the primers LW1306: 5′-GCATGAATTCTTCTACTTCATCATCCTCC-3′ (SEQ ID NO: 50) and LW1307: 5′-GCATGGATCCAAAGGCCATCACAACAAG-3′ (SEQ ID NO: 51). Primer LW1306 includes sequence identical to nucleotides 100- 118 of SEQ ID NO: 11 (underlined), preceded by an EcoRI site. Primer LW1307 includes sequence complementary to nucleotides 361-343 of SEQ ID NO: 11 (underlined), preceded by a BamHI restriction site. The amplification product was digested with EcoRI and BamHI, and then subcloned into an EcoRI- and BamHI-digested pBluescript II vector (Stratagene). The 261 amplified and subcloned basepairs correspond to nucleotides 100 to 361 of SEQ ID NO: 11.

The subcloned CON197-Bluescript construct was used to generate strand-specific probes for the in situ hybridization experiments. The construct was linearized with BamHI, for labeling with T7 polymerase (sense), or EcoRI, for T3 polymerase (antisense), and used as a template for in vitro transcription of sense and antisense cRNA riboprobes. The riboprobes were labeled with ³⁵S-UTP to yield a specific activity of 0.51×10⁶ cpm/pmol for antisense and 0.432×10⁶ cpm/pmol for sense probe. Hybridization with the riboprobes and subsequent washing of the slides was carried out as described above for CON193 in Example 3A.

Specific labeling with the antisense probe showed wide spread distribution of CON197 mRNA in the rat brain. Labelled regions included neo and allo cortex, piriform cortex, neostriatum, thalamic nuclei, hypothalamic nuclei, hippocampus, amygdala, cerebellum, and the olfactory bulb. The specificity of labeling was confirmed by microscopic analysis of emulsion coated sections. These sections revealed that the autoradiographic grains generated by the antisense probe were distributed over cell bodies rather than trapped between cell bodies. Sense probe did not generate specific labeling.

The observed regional distribution of CON197 mRNA provides a therapeutic indication for natural ligands for CON197 as well as modulators of CON197 activity, such as anti-CON197 antibody substances or small molecules that agonize or antagonize ligand-mediated CON197 signalling. In particular, the expression pattern provides an indication that such molecules will have utility for treating neurological and/or psychiatric diseases, including but not limited to dementia, schizophrenia, depression, anxiety, bipolar disease, migraine. Parkinson's disease, affective disorders. Alzheimer's disease. senile dementia, attention deficit hyperactivity disorder/attention deficit disorder (ADHD/ADD), epilepsy, neuritis, neurasthenia, neuropathy, neuroses, and the like. Use of CON197 modulators, including CON197 ligands and anti-CON197 antibodies, to treat individuals having such disease states is intended as an aspect of the invention. Such modulators are administered by any means effective to safely deliver the modulators to the CON197expressing cells, including but not limited to oral administration, inhalation, or injection of compositions comprising the modulators in a pharmaceutically acceptable diluent, adjuvant, or carrier. Efficacy of treatment can initially be determined in any accepted animal model that provides a biochemical or behavioral marker that correlates with disease severity or treatment efficacy.

G. CON202

A 272 bp fragment of CON202 containing EcoRI and BamHI restriction sites was amplified from the full-length clone by PCR, using the primers LW1310 GCATGAATTCGCAGAAGAAGGCTATTGG (SEQ ID NO: 56) and LW1311 GCATGGATCCGCAGTAAAGAAGGGTTGTG (SEQ ID NO: 57). The amplification product was digested with EcoRI and BamHI, and then subcloned into a pBluescript II vector (Strategene) that was digested with EcoRI and BamHI. The 272 amplified and subcloned basepairs correspond to nucleotides 1065 to 1336 of SEQ ID NO: 13.

The subcloned CON202-Bluescript construct was used to generate strand-specific probes for the in situ hybridization experiments. The construct was linearized with BamHI, for labeling with T7 polymerase (sense), or EcoRI, for T3 polymerase (antisense), and used as a template for in vitro transcription of sense and antisense cRNA riboprobes. The riboprobes were labeled with ³⁵S-UTP to yield a specific activity of 4.7×10⁵ cpm/pmol for antisense and 4.3×10⁵ cpm/pmol for sense probe. Hybridization with the riboprobes and subsequent washing of the slides was carried out as described above for CON193 in Example 3A.

Specific labeling with the antisense probe showed wide spread distribution of CON202 mRNA in the rat brain. Labelled regions included the cortical regions, lateral olfactory nuclei, hippocampus, subthalamic nucleus, and at a lower level, the nigra-pars compacta.

The observed regional distribution of CON202 mRNA provides a therapeutic indication for natural ligands for CON202 as well as modulators of CON202 activity, such as anti-CON202 antibody substances or small molecules that agonize or antagonize ligand-mediated CON202 signaling. In particular, the expression pattern provides an indication that such molecules will have utility for treating neurological and/or psychiatric diseases, including but not limited to schizophrenia, affective disorders, attention deficit hyperactivity disorder/attention deficit disorder, depression, anxiety, bipolar disease, epilepsy, neuritis, neurasthenia, neuropathy, neuroses, Alzheimer's disease, Parkinson's disease, migraine, senile dementia and the like. Use of CON202 modulators, including CON202 ligands and anti-CON202 antibodies, to treat individuals having such disease states is intended as an aspect of the invention. Such modulators are administered by any means effective to safely deliver the modulators to the CON202-expressing cells, including but not limited to oral administration, inhalation, or injection of compositions comprising the modulators in a pharmaceutically acceptable diluent, adjuvant, or carrier. Efficacy of treatment can initially be determined in any accepted animal model that provides a biochemical or behavioral marker that correlates with disease severity or treatment efficacy.

H. CON222

A 264 bp fragment of CON222 containing EcoRI and BamHI restriction sites was amplified from the full-length clone by PCR, using the primers LW1472 (5′GCATGAATTCTGCCATGTCAATCATTTCTCTC3′; SEQ ID NO: 62, EcoRI site is underlined) and LW1473 (5′GCATGGATCCGTTCTGCATTTTCCAGGTCTC3′; SEQ ID NO: 63, BamHI site is underlined). The amplification product was digested with EcoRI and BamHI, and then subcloned into a predigested pBluescript II vector (Stratagene). The 264 amplified and subcloned basepairs correspond to nucleotides 237 to 500 of SEQ ID NO: 15.

The subcloned CON222-Bluescript construct was used to generate strand-specific probes for the in situ hybridization experiments. The construct was linearized with BamHI, for labeling with T7 polymerase (sense), or EcoRI, for T3 polymerase (antisense), and used as a template for in vitro transcription of sense and antisense cRNA riboprobes. The riboprobes were labeled with ³⁵S-UTP to yield a specific activity of 4.25×10⁵ cpm/pmol for antisense and 3.9×10⁵ cpm/pmol for sense probe. Hybridization with the riboprobes and subsequent washing of the slides was carried out as described above for CON193 in Example 3A.

Specific labeling with the antisense probe showed wide spread distribution of CON222 MRNA in the rat brain. Labelled regions included the cortical regions, piriform cortex, stratum, hippocampus, thalamus, hypothalamus, dorsal raphe, and habenula.

The observed regional distribution of CON222 mRNA provides a therapeutic indication for natural ligands for CON222 as well as modulators of CON222 activity, such as anti-CON222 antibody substances or small molecules that agonize or antagonize ligand-mediated CON222 signaling. In particular, the expression pattern provides an indication that such molecules will have utility for treating neurological and/or psychiatric diseases, including but not limited to schizophrenia, affective disorders, attention deficit hyperactivity disorder/attention deficit disorder, depression, anxiety, bipolar disease, epilepsy, neuritis, neurasthenia, neuropathy, neuroses, Alzhemeimer's disease, Parkinson's Disease, migraine,senile dementia, and the like. Use of CON222 modulators, including CON222 ligands and anti-CON222 antibodies, to treat individuals having such disease states is intended as an aspect of the invention. Such modulators are administered by any means effective to safely deliver the modulators to the CON222-expressing cells, including but not limited to oral administration, inhalation, or injection of compositions comprising the modulators in a pharmaceutically acceptable diluent, adjuvant, or carrier. Efficacy of treatment can initially be determined in any accepted animal model that provides a biochemical or behavioral marker that correlates with disease severity or treatment efficacy.

I. CON215

A 261 bp fragment of CON215 containing EcoRI and BamHI restriction sites was amplified from the full-length clone by PCR, using the primers LW1411: 5′-GCATGAATTCTGCCAAACATCATCCTGAC-3′ (SEQ ID NO: 64) and LW1412: 5′-GCATGGATCCTACACAGCCACAACAACCC-3′ (SEQ ID NO: 65). Primer LW1411 contains an EcoRI site (underlined) followed by sequence identical to CON215 coding nucleotides 521-537, which correspond to positions 533-549 of SEQ ID NO: 17. Primer LW1412 contain a BamHI site (underlined) followed by sequence complementary to CON215 coding nucleotides 764-781, which correspond to positions 776-793 of SEQ ID NO: 17. The amplification product was digested with EcoRI and BamHI, and then subcloned into an EcoRI- and BamHI-digested pBluescript II vector (Stratagene). The 261 amplified and subcloned basepairs correspond to nucleotides 521 to 781 of SEQ ID NO: 17.

The subcloned CON215-Bluescript construct was used to generate strand-specific probes for the in situ hybridization experiments. The construct was linearized with BamHI, for labeling with T7 polymerase (sense), or EcoRI, for T3 polymerase (antisense), and used as a template for in vitro transcription of sense and antisense cRNA riboprobes. The riboprobes were labeled with ³⁵S-UTP to yield a specific activity of 48.03×10⁶ cpm/pmol for antisense and 48.09×10⁶ cpm/pmol for sense probe. Hybridization with the riboprobes and subsequent washing of the slides was carried out as described above for CON193 in Example 3A.

Subsequently, the slides were exposed to Kodak BioMax MR-1 film. After 9 days of exposure, the film was developed. Slides containing sections that showed a hybridization signal on film autoradiograms were coated with Kodak NTB-2 nuclear track emulsion and stored in the dark for 25 days. The slides were then developed as described above for CON193 in Example 3A.

Specific labeling with the antisense probe showed distribution of CON215 mRNA in the rat brain in limbic endocrine and motor circuits. Specifically, CON215 mRNA was present in the cortex, hippocampus, and red nucleus. The specificity of labeling was confirmed by microscopic analysis of emulsion coated sections. These sections revealed that the autbradiographic grains generated by the antisense probe were distributed over cell bodies rather than trapped between cell bodies. Sense probe did not generate specific labeling.

The observed regional distribution of CON215 mRNA provides a therapeutic indication for natural ligands for CON215 as well as modulators of CON215 activity, such as anti-CON215 antibody substances or small molecules that agonize or antagonize ligand-mediated CON1215 signaling. In particular, the expression pattern provides an indication that such molecules will have utility for treating neurological and/or psychiatric diseases, including but not limited to schizophrenia, depression, anxiety, bipolar disease, epilepsy, migraine, attention deficit (with or without hyperactive disorder), neuritis, neuasthenia, neuropathy, neuroses, Parkinson's disease, dementia, obesity, and the like. Use of CON215 modulators, including CON215 ligands and anti-CON215 antibodies, to treat individuals having such disease states is intended as an aspect of the invention.

Such modulators are administered by any means effective to safely deliver the modulators to the CON215-expressing cells, including but not limited to oral administration, inhalation, or injection of compositions comprising the modulators in a pharmaceutically acceptable diluent, adjuvant, or carrier. Efficacy of treatment can initially be determined in any accepted animal model that provides a biochemical or behavioral marker that correlates with disease severity or treatment efficacy.

J. CON217

Two oligonucleotides were designed based on SEQ ID NO: 19 and obtained from Sigma-Genosys (St. Louis, Mo.) to use as probes for in situ hybridization. The first oligonucleotide, designated 217A, has the sequence 5′TAGGTCGGTAGTCAGGACACGGGAGAACAGAACTGTTGGTTGA3′ (SEQ ID NO: 68) which is complementary to nucleotides 102 to 60 of SEQ ID NO: 19. The second oligonucleotide, designated 217B, has the sequence 5′GCCCCTGTGGCGGTTTAGATCCAGAATGCCCATTTTCTGTTCCATCTAACCA3′ (SEQ ID NO: 69) which corresponds to the complement of nucleotides 1530 to 1479 of SEQ ID NO: 17. Both oligonucleotides, 217A and 217B, were reconstituted with 1×TE buffer to a concentration of 20 pMol/ml and labeled with ³³P-dATP to yield a specific activity of 2.08×10⁶ and 1.53×10⁶ cpm/ml, respectively.

Hybridization was carried out at 37° C. overnight as described above for CON193 in Example 3A. Following the hybridizations, the coverslips were washed off the slides with 1×SSC for 45 minutes. The slides were then washed for 20 minutes at room temperature in 1×SSC followed by three high stringency washes in 1×SSC at 65° C. After washing, the slides were dehydrated with 70%, 95%, and 100% ethanol containing 0.3 mM NH₄OAc, air-dried, and exposed to Kodak BioMax MR-1 film. After 21 days of exposure, the film was developed. Based on these results, sections that showed a hybridization signal on film autoradiography were coated with Kodak NTB-2 nuclear track emulsion and stored in the dark for 42 days. The slides were then developed and counterstained with hematoxylin. Emulsion-coated sections were analyzed microscopically to determine the specificity of labeling. The signal was judged to be specific if autoradiographic grains (generated by antisense probe hybridization) were associated clearly with crystal violet stained cell bodies. Autoradiographic grains found between cell bodies were deemed nonspecific.

Specific labeling with the antisense probe showed wide spread distribution of CON217 mRNA in the rat brain. Labelled regions included the cortex, piriform cortex, hippocampus, cerebellum, medulla, spinal cord, temporal lobe, putamen, substantia nigra and thalamus.

The observed regional distribution of CON217 mRNAs provide a therapeutic indication for natural ligands for these G protein-coupled receptors as well as modulators of their activity, such as anti-CON217 antibody substances or small molecules that mimic, agonize or antagonize ligand-mediated CON217 signaling. In particular, the expression patterns provide an indication that such molecules will have utility for treating neurological and/or psychiatric diseases, including but not limited to schizophrenia, affective disorders, attention deficit hyperactivity disorder/attention deficit disorder, depression anxiety, bipolar disease, epilepsy, neuritis, neurasthenia, neuropathy, neuroses, Alzhemeimer's disease. Parkinson's Disease, migraine, senile dementia, and the like. Use of CON217 polypeptide modulators, including CON217 ligands and anti-CON217 polypeptide antibodies, to treat individuals having such disease states is intended as an aspect of the invention. Such modulators are administered by any means effective to safely deliver the modulators to the GPCR polypeptide-expressing cells, including but not limited to oral administration, inhalation, or injection of compositions comprising the modulators in a pharmaceutically acceptable diluent, adjuvant, or carrier. Efficacy of treatment can initially be determined in any accepted animal model that provides a biochemical or behavioral marker that correlates with disease severity or treatment efficacy.

### EXAMPLE 4

Recombinant Expression of GPCR Polypeptides in Eukaryotic Host Cells

To produce GPCR protein, a GPCR polypeptide-encoding polynucleotide is expressed in a suitable host cell using a suitable expression vector, using standard genetic engineering techniques. For example, one of the GPCR polypeptide-encoding sequences described in Example 1 (such as SEQ ID NOS: 1, 3, 5, 7, 9, 11, 13, 15, 17 or 19) is subcloned into the commercial expression vector pzeoSV2 (Invitrogen, San Diego, Calif.) and transfected into Chinese Hamster Ovary (CHO) cells (ATCC CRL-1781) using the transfection reagent fuGENE 6 (Boehringer-Mannheim) and the transfection protocol provided in the product insert. Additional eukaryotic cell lines, such as African Green Monkey Kidney cells (COS-7, ATCC CRL-1651) or Human Kidney cells (HEK 293, ATCC CRL-1573), may be used as well. Cells stably expressing a GPCR polypeptide (e.g., CON193, CON166, CON103, CON203, CON198, CON197, CON202, CON222, CON215, or CON217) are selected by growth in the presence of 100 mg/ml zeocin (Stratagene, LaJolla, Calif.). Optionally, GPCR polypeptide is purified from the cells using standard chromatographic techniques. To facilitate purification, antisera is raised against one or more synthetic peptide sequences that correspond to portions of the GPCR amino acid sequence, and the antisera is used to affinity purify GPCR polypeptides. The GPCR gene also may be expressed in frame with a tag sequence (e.g., polyhistidine, hemaggluttinin, FLAG) to facilitate purification. Moreover, it will be appreciated that many of the uses for GPCR polypeptides, such as assays described below, do not require purification of GPCR polypeptides from the host cell.

### EXAMPLE5

Antibodies to GPCR Polypeptides

Standard techniques are employed to generate polyclonal or monoclonal antibodies to the GPCR receptors (e.g., CON193, CON166, CON103, CON203, CON198, CON197, CON202, CON222, CON215, or CON217), and to generate useful antigen-binding fragments thereof or variants thereof, including “humanized” variants. Such protocols can be found, for example, in Sambrook et al., Molecular Cloning: a Laboratory Manual. Second Edition, Cold Spring Harbor, N.Y.: Cold Spring Harbor Laboratory (1989); Harlow et al. (Eds), Antibodies A Laboratory Manual; Cold Spring Harbor Laboratory; Cold Spring Harbor, N.Y. (1988); and other documents cited below. In one embodiment, recombinant GPCR polypeptides (or cells or cell membranes containing such polypeptides) of the invention are used as an antigen to generate the antibodies. In another embodiment, one or more peptides having amino acid sequences corresponding to an immunogenic portion of a GPCR polypeptide (e.g., 6, 7, 8, 9, 10, 11, 12, 13, 14, 15, 16, 17, 18, 19, 20, or more amino acids) are used as antigen. Peptides corresponding to extracellular portions of GPCR polypeptides, especially hydrophilic extracellular portions, are preferred. The antigen may be mixed with an adjuvant or linked to a hapten to increase antibody production.

A. Polyclonal or Monoclonal Antibodies

As one exemplary protocol, a recombinant GPCR polypeptide or synthetic fragment thereof is used to immunize a mouse for generation of monoclonal antibodies (or larger mammal, such as a rabbit, for polyclonal antibodies). To increase antigenicity, peptides are conjugated to Keyhole Lympet Hemocyanine (Pierce), according to the manufacturer's recommendations. For an initial injection, the antigen is emulsified with Freund's Complete Adjuvant and injected subcutaneously. At intervals of two to three weeks, additional aliquots of GPCR antigen are emulsified with Freund's Incomplete Adjuvant and injected subcutaneously. Prior to the final booster injection, a serum sample is taken from the immunized mice and assayed by Western blot to confirm the presence of antibodies that immunoreact with GPCR polypeptide. Serum from the immunized animals may be used as a polyclonal antisera or used to isolate polyclonal antibodies that recognize GPCR polypeptide. Alternatively, the mice are sacrificed and their spleen removed for generation of monoclonal antibodies.

To generate monoclonal antibodies, the spleens are placed in 10 ml serum-free RPMI 1640, and single cell suspensions are formed by grinding the spleens in serum-free RPMI 1640, supplemented with 2 mM L-glutamine, 1 mM sodium pyruvate, 100 units/ml penicillin, and 100 μg/ml streptomycin (RPMI) (Gibco, Canada). The cell suspensions are filtered and washed by centrifugation and resuspended in serum-free RPMI. Thymocytes taken from three naive Balb/c mice are prepared in a similar manner and used as a Feeder Layer. NS-1 myeloma cells, kept in log phase in RPMI with 10% fetal bovine serum (FBS) (Hyclone Laboratories, Inc., Logan, Utah) for three days prior to fusion, are centrifuged and washed as well.

To produce hybridoma fusions, spleen cells from the immunized mice are combined with NS-1 cells and centrifuged, and the supernatant is aspirated. The cell pellet is dislodged by tapping the tube, and 2 ml of 37° C. PEG 1500 (50% in 75 mM Hepes, pH 8.0) (Boehringer Mannheim) is stirred into the pellet, followed by the addition of serum-free RPMI. Thereafter, the cells are centrifuged and resuspended in RPMI containing 15% FBS, 100 μM sodium hypoxanthine, 0.4 μM aminopterin, 16 μM thymidine (HAT) (Gibco), 25 units/ml of IL-6 (Boehringer Mannheim) and 1.5×10⁶ thymocytes/ml and plated into 10 Corning flat-bottom 96-well tissue culture plates (Coming, Corning N.Y.).

On days 2, 4, and 6, after the fusion, 100 μl of medium is removed from the wells of the fusion plates and replaced with fresh medium. On day 8, the fusions are screened by ELISA, testing for the presence of mouse 1 gG that binds to a GPCR polypeptide. Selected fusion wells are further cloned by dilution until monoclonal cultures producing anti-GPCR polypeptide antibodies are obtained.

B. Humanization of Anti-GPCR Monoclonal Antibodies

The expression patterns of GPCR polypeptides as reported herein and the proven track record of GPCR's as targets for therapeutic intervention suggest therapeutic indications for GPCR polypeptide inhibitors (antagonists). GPCR polypeptide-neutralizing antibodies comprise one class of therapeutics useful as antagonists. Following are protocols to improve the utility of anti-GPCR polypeptide monoclonal antibodies as therapeutics in humans, by “humanizing” the monoclonal antibodies to improve their serum half-life and render them less immunogenic in human hosts (i.e., to prevent human antibody response to non-human anti-GPCR polypeptide antibodies).

The principles of humanization have been described in the literature and are facilitated by the modular arrangement of antibody proteins. To minimize the possibility of binding complement, a humanized antibody of the IgG4 isotype is preferred.

For example, a level of humanization is achieved by generating chimeric antibodies comprising the variable domains of non-human antibody proteins of interest with the constant domains of human antibody molecules. (See, e.g., Morrison and Oi, Adv. Immunol., 44:65-92 (1989). The variable domains of GPCR-neutralizing anti-GPCR antibodies are cloned from the genomic DNA of a B-cell hybridoma or from cDNA generated from mRNA isolated from the hybridoma of interest. The V region gene fragments are linked to exons encoding human antibody constant domains, and the resultant construct is expressed in suitable mammalian host cells (e.g., myeloma or CHO cells).

To achieve an even greater level of humanization, only those portions of the variable region gene fragments that encode antigen-binding complementarity determining regions (“CDR”) of the non-human monoclonal antibody genes are cloned into human antibody sequences. [See, e.g., Jones et al., Nature, 321:522-525 (1986); Riechmann et al., Nature, 332:323-327 (1988); Verhoeyen et al., Science, 739:1534-36 (1988); and Tempest et al., Bio/Technology, 9:266-71 (1991). If necessary, the P-sheet framework of the human antibody surrounding the CDR3 regions also is modified to more closely mirror the three dimensional structure of the antigen-binding domain of the original monoclonal antibody. (See Kettleborough et al., Protein Engin., 4:773-783 (1991); and Foote et al., J. Mol. Biol., 224:487-499 (1992).

In an alternative approach, the surface of a non-human monoclonal antibody of interest is humanized by altering selected surface residues of the non-human antibody, e.g., by site-directed mutagenesis, while retaining all of the interior and contacting residues of the non-human antibody. See Padlan, Molecular Immunol., 28(4/5):489-98 (1991).

The foregoing approaches are employed using GPCR-neutralizing anti-GPCR monoclonal antibodies and the hybridomas that produce them to generate humanized GPCR-neutralizing antibodies useful as therapeutics to treat or palliate conditions wherein GPCR expression or ligand-mediated GPCR signaling is detrimental.

C. Human GPCR-Neutralizing Antibodies From Phage Display

Human GPCR-neutralizing antibodies are generated by phage display techniques such as those described in Aujame et al., Human Antibodies, 8(4):155-168 (1997); Hoogenboom, TIBTECH, 15:62-70 (1997); and Rader et al., Curr. Opin. Biotechnol., 8:503-508 (1997), all of which are incorporated by reference. For example, antibody variable regions in the form of Fab fragments or linked single chain Fv fragments are fused to the amino terminus of filamentous phage minor coat protein pIII. Expression of the fusion protein and incorporation thereof into the mature phage coat results in phage particles that present an antibody on their surface and contain the genetic material encoding the antibody. A phage library comprising such constructs is expressed in bacteria, and the library is panned (screened) for GPCR-specific phage-antibodies using labelled or immobilized GPCR polypeptide as antigen-probe.

D. Human GPCR-Neutralizing Antibodies From Transgenic Mice

Human GPCR-neutralizing antibodies are generated in transgenic mice essentially as described in Bruggemann and Neuberger, Immunol. Today, 17(8):391-97 (1996) and Bruggemann and Taussig, Curr. Opin. Biotechnol. 8:455-58 (1997). Transgenic mice carrying human V-gene segments in germline configuration and that express these transgenes in their lymphoid tissue are immunized with a GPCR composition using conventional immunization protocols. Hybridomas are generated using B cells from the immunized mice using conventional protocols and screened to identify hybridomas secreting anti-GPCR human antibodies (e.g., as described above).

### EXAMPLE 6

Assays to Identify Modulators of GPCR Polypeptide Activity

Set forth below are assays for identifying modulators (agonists and antagonists) of GPCR polypeptide activity. Among the modulators that can be identified by these assays include natural ligand compounds of the receptor; synthetic analogs and derivatives of natural ligands; antibodies, antibody fragments, and/or antibody-like compounds derived from natural antibodies or from antibody-like combinatorial libraries; and/or synthetic compounds identified through high throughput screening of libraries; and the like. All modulators that bind GPCR polypeptide are useful for identifying GPCR polypeptide in tissue samples (e.g., for diagnostic purposes, pathological purposes, and the like). Agonist and antagonist modulators are useful for up-regulating and down-regulating GPCR polypeptide activity, respectively, to treat disease states characterized by abnormal levels of GPCR polypeptide activity. GPCR polypeptide binding molecules also may be used to deliver a therapeutic compound or a label to cells that express GPCR polypeptide (e.g., by attaching the compound or label to the binding molecule). The assays may be performed using single putative modulators, and/or may be performed using a known agonist in combination with candidate antagonists (or visa versa). Performance of the assays using any of the GPCR polypeptides of the invention described herein (e.g., CON193, CON166, CON103, CON203, CON198, CON197, CON202. CON222, CON215, or CON217) is contemplated. It will be appreciated that co-transfecting cells with two or more of the receptors for simultaneous screening also is possible.

A. cAMP Assays

In one type of assay, levels of cyclic adenosine monophosphate (cAMP) are measured in GPCR-transfected cells that have been exposed to candidate modulator compounds. Protocols for cAMP assays have been described in the literature. [See, e.g., Sutherland et al., Circulation, 37: 279 (1968); Frandsen, E. K. and Krishna, G, Life Sciences, 18: 529-541 (1976); Dooley et al., Journal of Pharmacology and Experimental Therapeutics, 283 (2): 735-41 (1997); and George et al., Journal of Biomolecular Screening, 2 (4): 235-40 (1997).] An exemplary protocol for such an assay, using an Adenylyl Cyclase Activation FlashPlate® Assay from NEN™ Life Science Products, is set forth below.

Briefly, the GPCR coding sequence (e.g., a cDNA or intronless genomic DNA) is subcloned into a commercial expression vector, such as pzeoSV2 (Invitrogen, San Diego, Calif.), and transiently transfected into Chinese Hamster Ovary (CHO) cells using known methods, such as the transfection reagent FuGENE 6 (Boehringer-Mannheim) and the transfection protocol provided in the product insert.

The transfected CHO cells are seeded into the 96 well microplates from the FlashPlate® assay kit, which are coated with solid scintillant to which antisera to cAMP has been bound. For a control, some wells are seeded with wild type (untransfected) CHO cells. Other wells on the plate receive various amounts of cAMP standard solution for use in creating a standard curve.

One or more test compounds are added to the cells in each well, with water and/or compound-free media/diluent serving as a control. After treatment, cAMP is allowed to accumulate in the cells for exactly 15 minutes at room temperature. The assay is terminated by the addition of lysis buffer containing [¹²⁵I]-labelled cAMP, and the plate is counted using a Packard Topcount™ 96-well microplate scintillation counter. Unlabelled cAMP from the lysed cells (or from standards) competes with the fixed amounts of [¹²⁵I]-cAMP for antibody bound to the plate. A standard curve is constricted, and cAMP values for the unknowns are obtained by interpolation. Changes in intracellular cAMP level of the cells in response to exposure to a test compound are indicative of GPCR polypeptide modulating activity. Modulators that act as agonists at receptors which couple to the Gs subtype of G-proteins will stimulate production of cAMP, leading to a measurable 3-10 fold increase. Receptor agonists which couple to the Gi/o subtype of G-proteins will inhibit forskolin-stimulated cAMP production, leading to a measurable decrease of 50-100%. Modulators that act as inverse agonists will reverse these effects at receptors that are either constitutively active or activated by known agonists.

B. Aeguorin Assays

In another assay cells (e.g., CHO cells) are transiently co-transfected with both a GPCR expression construct and a construct that encodes the photoprotein apoaequorin. In the presence of the cofactor coelenterazine, apoaequorin will emit a measurable luminescence that is proportional to the amount of intracellular (cytoplasmic) free calcium. [See generally Cobbold P. H. and Lee, J. A. C. “Aequorin measurements of cytoplasmic free calcium. In: McCormack J. G. and Cobbold P. H., eds., Cellular Calcium: A Practical Approach. Oxford:IRL Press (1991); Stables et al., Analytical Biochemistry, 252: 115-26 (1997); and Haugland, R. P. Handbook of Fluorescent Probes and Research Chemicals. Sixth edition. Eugene Oreg.: Molecular Probes (1996).]

In one exemplary assay, a GPCR-encoding polynucleotide is subcloned into the commercial expression vector pzeoSV2 (Invitrogen, San Diego, Calif.) and transiently co-transfected along with a construct that encodes the photoprotein apoaequorin (Molecular Probes, Eugene, Oreg.) into CHO cells using the transfection reagent FuGENE 6 (Boehringer-Mannheim) and the transfection protocol provided in the product insert.

The cells are cultured for 24 hours at 37° C. in αMEM (Gibco/BRL, Gaithersburg, Md.) supplemented with 10% FBS, 2 mM glutamine, 10 U/ml of penicillin and 10 μg/ml of streptomycin. Subsequently, the media is changed to serum-free αMEM containing 5 μM coelenterazine (Molecular Probes, Eugene, Oreg.), and the cells are cultured for two additional hours at 37° C. Cells are then detached from the plate using VERSEN (Gibco/BRL). washed and resuspended at 2×10⁵ cells/ml in serum-free αMEM.

Dilutions of candidate GPCR modulator drugs are prepared in serumfree αMEM and dispensed into wells of an opaque 96-well assay plate, 50 μl/well. Plates are loaded onto an MLX microtiter plate luminometer (Dynex Technologies, Inc., Chantilly, Va.). The instrument is programmed to dispense 50 μl of cell suspension into each well, one well at a time, and immediately read luminescence for 15 seconds. Dose-response curves for the modulator candidates are constructed using the area under the curve for each light signal peak. Data are analyzed with SlideWrite, using the equation for 1-site ligand, and EC₅₀ values are obtained. Changes in luminescence caused by the drugs are considered indicative of modulatory activity. Modulators that act as receptor agonists which couple to the Gq subtype of G-proteins give an increase in luminescence of up to 100 fold. Modulators that act as inverse agonists will reverse this effect at receptors that are either constitutively active or activated by known agonists.

C. Luciferase Reporter Gene Assay

The photoprotein luciferase provides another useful tool for assaying for modulators of GPCR activity. Cells (e.g., CHO cells or COS 7 cells) are transiently co-transfected with both a GPCR expression construct (e.g., GPCR-encoding sequence in pzeoSV2 (Invitrogen, San Diego, Calif.)) and a reporter construct which includes a gene for the luciferase protein downstream from a transcription factor, either cAMP-response element (CRE), AP-1, or NF kappa B. Agonist binding to receptors coupled to the Gs subtype of G-proteins leads to increases in cAMP, activating the CRE transcription factor and resulting in expression of the luciferase gene. Agonist binding to receptors coupled to the Gq subtype of G-protein leads to production of diacylglycerol that activates protein kinase C. As a result, the AP-1 or NF kappa B transcription factors are activated which stimulate expression of the luciferase gene. Expression levels of luciferase reflect the activation status of the signaling events. [See generally George et al., Journal of Biomolecular Screening, 2(4): 235-40 (1997); and Stratowa et al., Current Opinion in Biotechnology, 6: 574-81 (1995).] Luciferase activity may be quantitatively measured using, e.g., luciferase assay reagents that are commercially available from Promega (Madison, Wis.).

In one exemplary assay, CHO cells are plated in 24-well culture dishes at a density of 100,000 cells/well one day prior to transfection and cultured at 37° C. in αMEM (Gibco/BRL, Gaithersburg, Md.) supplemented with 10% FBS, 2 mM glutamine, 10 U/ml penicillin and 10 μg/ml streptomycin. Cells are transiently co-transfected with both a GPCR expression construct and a reporter construct containing the luciferase gene. The reporter plasmids CRE-luciferase, AP-1-luciferase and NF kappa B-luciferase may be purchased from Stratagene (LaJolla, Calif.). Transfections are performed using FuGENE 6 transfection reagent (Boehringer-Mannheim), and the protocol provided in the product insert. Cells transfected with the reporter construct alone are used as a control. Twenty-four hours after transfection, cells are washed once with phosphate buffered saline (PBS) pre-warmed to 37° C. Serum-free αMEM is then added to the cells either alone (control) or with one or more candidate modulators and the cells are incubated at 37° C. for five hours. Thereafter, cells are washed once with ice cold PBS and lysed by the addition of 100 μl of lysis buffer/well (from luciferase assay kit, Promega, Madison, Wis.). After incubation for 15 minutes at room temperature, 15 μl of the lysate is mixed with 50 μl substrate solution (Promega) in an opaque white 96-well plate, and the luminescence is read immediately on a Wallace model 1450 MicroBeta scintillation and luminescence counter (Wallace Instruments, Gaithersburg, Md.).

Differences in luminescence in the presence versus the absence of a candidate modulator compound are indicative of modulatory activity. Receptors that are either constitutively active or activated by agonists give a 3-20 fold stimulation of luminescence compared to cells transfected with the reporter gene alone. Modulators that act as inverse agonists will reverse this effect.

D. Intracellular Calcium Measurement Using FLIPR

Changes in intracellular calcium levels are another recognized indicator of G protein-coupled receptor activity, and such assays can be employed to evaluate modulators of GPCR activity. For example, CHO cells stably transfected with a GPCR expression vector are plated at a density of 4×10⁴ cells/well in Packard black-walled 96-well plates specially designed to isolate fluorescent signal to individual wells. The cells are incubated for 60 minutes at 37° C. in modified Dulbecco's PBS (D-PBS) containing 36 mg/L of pyruvate and 1 g/L of glucose with the addition of 1% FBS and one of four calcium indicator dyes (Fluo-3™ AM, Fluo4™ AM, Calcium Green™-1 AM, or Oregon Green™ 488 BAPTA-1 AM) at a concentration of 4 μM. Plates are washed once with modified D-PBS without 1% FBS and incubated for 10 minutes at 37° C. to remove residual dye from the cellular membrane. In addition, a series of washes with modified D-PBS without 1% FBS is performed immediately prior to activation of the calcium response.

Calcium response is initiated by the addition of one or more candidate receptor agonist compounds, calcium ionophore A23187 (10 μM), or ATP (4 μM). Fluorescence is measured by Molecular Device's FLIPR with an argon laser, excitation at 488 nm. [See, e.g., Kuntzweiler et al., Drug Development Research, 44(1): 14-20 (1998).] The F-stop for the detector camera was set at 2.5 and the length of exposure was 0.4 milliseconds. Basal fluorescence of cells was measured for 20 seconds prior to addition of agonist, ATP, or A23187, and was subtracted from the response signal. The calcium signal is measured for approximately 200 seconds, taking readings every two seconds. Calcium ionophore and ATP increase the calcium signal 200% above baseline levels. In general, activated orphan GPCRs increase the calcium signal approximately 10-15% above baseline signal.

E. Mitogenesis Assay

In mitogenesis assays, the ability of candidate modulators to induce or inhibit GPCR-mediated cell growth is determined. [See, e.g., Lajiness et al., Journal of Pharmacology and Experimental Therapeutics, 267(3): 1573-81 (1993).]

For example, CHO cells stably expressing a GPCR are seeded into 96-well plates at a density of 5000 cells/well and grown at 37° C. in αMEM supplemented with 10% fetal calf serum. After 48 hours, the cells are rinsed twice with serum-free αMEM and 80 μl of fresh αMEM, or αMEM containing a known mitogen, is added along with 20 μl αMEM containing varying concentrations of one or more test compounds diluted in serum free media. As controls, some wells on each plate receive serum-free media alone, and some receive media containing 10% FBS. Untransfected cells or cells transfected with vector alone also may serve as controls.

After culture for 16-18 hours, 1 μCi/well of [³H]-thymidine (2 Ci/mmol; cpm) is added to the wells and cells are incubated for an additional 2 hours at 37° C. The cells are trypsinized and harvested onto filter mats with a cell harvester (Tomtec) and the filters are counted in a Betaplate counter. The incorporation of ³H-thymidine in serum-free test wells is compared to the results achieved in cells stimulated with serum. Use of multiple concentrations of test compounds permits creation and analysis of dose-response curves using the non-linear, least squares fit equation: A=B×[C/(D+C)]+G where A is the percent of serum stimulation; B is the maximal effect minus baseline; C is the EC₅₀; D is the concentration of the compound; and G is the maximal effect. Parameters B, C and G are determined by Simplex optimization.

Agonists that bind to the receptor are expected to increase [³H]-thymidine incorporation into cells, showing up to 80% of the response to serum. Antagonists that bind to the receptor will inhibit the stimulation seen with a known agonist by up to 100%.

F. [³⁵S]GTPγS Binding Assay

Because G protein-coupled receptors signal through intracellular “G proteins” whose activity involves GTP/GDP binding and hydrolysis. Another indicator of GPCR modulator activity is measuring binding of the non-hydrolyzable GTP analog [³⁵S]GTPγS in the presence and absence of putative modulators. [See, e.g., Kowal, et al., Neuropharmacology, 37: 179-87 (1998).]

In one exemplary assay, cells stably transfected with a GPCR expression vector are grown in 10 cm dishes to subconfluence, rinsed once with 5 ml of ice cold Ca²⁺/Mg²⁺ free PBS, and scraped into 5 ml of the same buffer. Cells are pelleted by centrifugation (500×g, 5 minutes), resuspended in TEE buffer (25 mM Tris, 5 mM EDTA, 5 mM EGTA, pH 7.5) and frozen in liquid nitrogen. After thawing, the cells are homogenized using a dounce (one ml TEE per plate of cells). and centrifuged at 1,000×g for 5 minutes to remove nuclei and unbroken cells.

The homogenate supernatant is centrifuged at 20,000×g for 20 minutes to isolate the membrane fraction. The membrane pellet is then washed once with TEE and resuspended in binding buffer (20 mM HEPES, pH 7.5, 150 mM NaCl, 10 mM MgCl₂, 1 mM EDTA). The resuspended membranes can be frozen in liquid nitrogen and stored at −70° C. until use.

Aliquots of cell membranes prepared as described above and stored at −70° C. are thawed, homogenized, and diluted to a concentration of 10-50 μg/ml in buffer containing 20 mM HEPES, 10 mM MgCl₂, 1 mM EDTA, 120 mM NaCl, 10 μM GDP, and 0.2 mM ascorbate. In a final volume of 90 μl, homogenates are incubated with varying concentrations of putative modulator compounds or 100 μM GTP for 30 minutes at 30° C. and then placed on ice. To each sample, 10 μl guanosine 5′-O-(3[³⁵S]thio) triphosphate (NEN, 1200 Ci/mmol), ([³⁵S]-GTPγS), was added to a final concentration of 100-200 pM. Samples are incubated at 30° C. for an additional 30 minutes. The reaction is then stopped by the addition of 1 ml of 10 mM HEPES, and 10 mM MgCl₂ (pH 7.4), at 4° C., and filtration.

Samples are filtered over Whatman GF/B filters. These filters are washed with 20 ml ice-cold 10 mM HEPES (pH 7.4) and 10 mM MgCl₂ and counted by liquid scintillation spectroscopy. Nonspecific binding of [³⁵S]-GTPγS is measured in the presence of 100 μM GTP and subtracted from the total. Compounds are selected that modulate the amount of [³⁵S]-GTPγS binding in the cells, compared to untransfected control cells. Activation of receptors by agonists gives up to a five-fold increase in [³⁵S]GTPγS binding. This response is blocked by antagonists.

G. MAP Kinase Activity Assay

Evaluation of MAP Kinase activity in cells expressing a GPCR provide another assay to identify modulators of GPCR activity. [See, e.g., Lajiness et al., Journal of Pharmacology and Experimental Therapeutics, 267(3): 1573-81(1993); and Boulton et al., Cell, 65: 663-75 (1991).]

In one embodiment. CHO cells stably transfected with a GPCR-encoding polynucleotide are seeded into 6 well plates at a density of 70,000 cells/well 48 hours prior to the assay. During this time, the cells are cultured at 37° C. in αMEM media supplemented with 10% FBS, 2 mM glutamine, 10 U/ml penicillin and 10 μg/ml streptomycin. The cells are serum starved for 1-2 hours prior to the addition of stimulants.

For the assay, the cells are treated with media alone or media containing a putative agonist or phorbal ester-myristyl acetate (PMA) as a positive control. After treatment, cells are incubated at 37° C. for varying times. To stop the reaction, the plates are placed on ice, the media is aspirated, and the cells are rinsed with 1 ml of ice-cold PBS containing 1 mM EDTA. Thereafter, 200 μl cell lysis buffer (12.5 mM MOPS (pH 7.3), 12.5 mM β-glycerophosphate, 7.5 mM MgCl₂, 0.5 mM EGTA, 0.5 mM sodium vanadate, 1 mM benzamidine, 1 mM dithiothreitol, 10 μg/ml leupeptin, 10 μg/ml aprotinin, 2 μg/ml pepstatin A, and .1 μM okadaic acid) is added to the cells. The cells are scraped from the plates and homogenized by 10 passages through a 23¾ gauge needle. The cytosol fraction is prepared by centrifugation at 20,000×g for 15 minutes.

Aliquots (5-10 μl containing 1-5 μg protein) of cytosols are mixed with 1 mM MAPK Substrate Peptide (APRTPGGRR; SEQ ID NO: 25); Upstate Biotechnology, Inc., N.Y.) and 50 μM [γ-³²P]ATP, (NEN, 3000 Ci/mmol) diluted to a final specific activity of ˜2000 cpm/pmol in a total volume of 25 μl. The samples are incubated for 5 minutes at 30° C., and reactions are stopped by spotting 20 μl on 2 cm² of Whatman P81 phosphocellulose paper. The filter squares are washed in 4 changes of 1% H₃PO₄, and the squares are counted by liquid scintillation spectroscopy. Equivalent cytosolic extracts are incubated without MAPK substrate peptide, and the cpm from these samples are subtracted from the matched samples with the substrate peptide. The cytosolic extract from each well is used as a separate point: Protein concentrations are determined by a dye binding protein assay (Bio-Rad). Agonist activation of the receptor is expected to result in up to a five fold increase in MAPK enzyme activity. This increase is blocked by antagonists.

H. [³H]Arachidonic Acid Release

The activation of GPCR's also has been observed to potentiate arachidonic acid release in cells, providing yet another useful assay for modulators of the activity of GPCR's of the present invention. [See, e.g., Kanterman et al., Molecular Pharmacology, 39: 364-9 (1991).] For example, CHO cells that are stably transfected with a GPCR expression vector are plated in 24-well plates at a density of 15000 cells/well and grown in αMEM media supplemented with 10% FBS, 2 mM glutamine, 10 U/ml penicillin and 10 μg/ml streptomycin for 48 hours at 37° C. before use. Cells of each well are labeled by incubation with [³H]arachidonic acid (Amersham Corp., 210 Ci/mmol) at 0.5 μCi/ml in 1 ml αMEM supplemented with 10 mM HEPES (pH 7.5), and 0.5% fatty-acid-free bovine serum albumin for 2 hours at 37° C. The cells are then washed twice with 1 ml of the same buffer.

Candidate modulator compounds are added in 1 ml of the same buffer, either alone or containing 10 μM ATP (Adenosine 5′-triphosphate) and the cells are incubated at 37° C. for 30 minutes. Buffer alone and mock transfected cells are used as controls. Samples (0.5 ml) from each well are counted by liquid scintillation spectroscopy. Agonists which activate the receptor will lead to potentiation of the ATP-stimulated release of [³H]-arachidonic acid. This potentiation is blocked by antagonists.

I. Extracellular Acidification Rate

In yet another assay, the effects of putative modulators of GPCR activity are assayed by monitoring extracellular changes in pH induced by the putative modulators. [See, e.g., Dunlop et al., Journal of Pharmacological and Toxicological Methods, 40(1): 47-55 (1998).]

CHO cells transfected with a GPCR expression vector are seeded into 12-mm capsule cups (Molecular Devices Corp.) at 4×10⁵ cells/cup in αMEM supplemented with 10% FBS, 2 mM 1-glutamine, 10 units/ml penicillin, and 10 μg/ml streptomycin. The cells are incubated in this media at 37° C. in 5% CO₂ for 24 hours.

Extracellular acidification rates are measured using a Cytosensor microphysiometer (Molecular Devices Corp.). The capsule cups are loaded into the sensor chambers of the microphysiometer and the chambers are perfused with running buffer (bicarbonate free αMEM supplemented with 4 mM 1-glutamine, 10 units/ml penicillin, 10 μg/ml streptomycin, 26 mM NaCl) at a flow rate of 100 μl/min. Agonists or other agents are diluted into the running buffer and perfused through a second fluid path. During each 60 second pump cycle, the pump is run for 38 seconds and is off for the remaining 22 seconds. The pH of the running buffer in the sensor chamber is recorded during the cycle from 43-58 seconds, and the pump is re-started at 60 seconds to start the next cycle. The rate of acidification of the running buffer during the recording time is calculated by the Cytosoft program. Changes in the rates of acidification are calculated by subtracting the baseline value (the average of 4 rate measurements immediately before addition of modulator candidates) from the highest rate measurement obtained after addition of a modulator candidate. The selected instrument detects 61 mV/pH unit. Modulators that act as agonists at the receptor result in an increase in the rate of extracellular acidification as.compared to the rate in the absence of agonist. This response is blocked by modulators which act as antagonists at the receptor.

### EXAMPLE 7

Luciferase Reporter Gene Assays

Luciferase reporter gene assays (essentially as described in Example 6) were carried out to measure signaling activity of the GPCR receptors when coupled to Gs, Gi or Gq G-proteins. Activation of Gs coupled receptors results in stimulation of intracellualar cAMP production which leads to activation of the transcription factor cyclic AMP response element (CRE). Therefore activation of Gs coupled receptors can be detected by measuring transcription and translation of the reporter gene CRE-luciferase. The level of expression of the CRE reporter gene is dependent on the intracellular level of cAMP. Similarily, activation of Gs, Gi or Gq coupled receptors will result in activation of the AP-1 transcription factor. Expression of the AP-1 transcription factor can be attributed to changes in cAMP levels and/or increases in the levels of intracellular calcium and therefore can be an indication of G-protein coupled receptor activation.

CHO 10001A cells (Gottesman et al., Somatic Cell Genetics 6: 45-61, 1980) were maintained in Minimal Essential Medium (MEM) supplemented with 10% FBS (Hyclone Laboratories, Inc., Logan, Utah) at 37° C. in an atmosphere of 5% CO₂. The cells were split 1:5 twice a week for maintence. Plasmids used in the experiments were propogated in E.coli strain DH5 (Gibco BRL) and purified using the Qiagen Maxi-prep plasmid purification system according to the manufacturer's instructions.

One day prior to transfection, 1×10⁵ CHO cells/well were plated on 24 well culture plates and allowed to adhere overnight. Each well on the plate was transfected with 0.5 μg of either AP-1 luciferase (Stratagene,, LaJolla, Calif.) or CRE luciferase plasmid alone or in combination with 0.125 μg of a GPCR plasmid (GPCR DNA inserted into the pCDNA3 vector form Invitrogen). Cell were transiently transfected with the commercially available transfection reagent FUGENE-6 according the manufacturer's instructions (Boehringer Mannheim, Indianapolis, Ind.).

Twenty-four hours after transfection, the cells were washed in PBS pre-warmed to 37° C. Agonists and antagonists were diluted in pre-warmed serum-free MEM, added to the transfected cells and incubated at 37° C., 5% CO₂ for 5 hours. Subsequently, the cells were washed once in ice cold PBS and lysed with the addition of 100 μl of lysis buffer (Promega) to each well. After a 15 minute incubation at room temperature, luciferase reporter gene activation was analyzed with the Luciferase Assay Reagents commercially available from Promega (Madison. Wis.). An alloquot of lysate (15 μl) was mixed with 50 μl of substrate solution in an opaque white 96 well plate. The luminescence from the plate was read in a Wallance 1450 MicroBeta scintillation and luminscence counter (Wallac Instruments, Gaithersburg, Md.). Constitutive GPCR activity was calculated as activity measured in GPCR transfected cells divided by activity measured in control cells (control cells=luciferase-transfected cells in the absence of GPCR plasmid). The measurements of GPCR constitutive activity (as a percentage of control measurements) are summarized in the table below:

| GPCR | CRE Activity | AP-1 Activity |
|---|---|---|
| CON193 | 128% | 100% |
| CON197 | 165% | 100% |
| CON198 | 178% | 146% |
| CON203 | 100% | 468% |
| CON215 | 173% | 307% |
| CON222 | 100% | 100% |
| CON202 | 135% | 336% |
| CON166 | 115% | 100% |
| CON217 | 211% | 100% |

These results provide useful information for designing screening assays to identify molecules (natural or artificial) that activate or inhibit the GPCR's of the invention. For example, compound libraries can be screened using the AP-1 luciferase (for CON198, CON203, CON215, or CON202) or the CRE-luciferase assay (for CON193, CON197, CON198, CON215, CON202, and CON166) to identify compounds which increase the signaling activity in GPCR polypeptide expressing cells as compared to receptor negative cells. The identified compounds may be useful for predicting endogenous ligands for the GPCR polypeptides, for measuring the physiological effects of GPCR activation in animal models, and for designing therapeutics to modulate GPCR activity to treat disease states.

### EXAMPLE 8

### Chromosomal Localization of GPCR

The following example pertains to chromosomal localization of GPCR genes of the present invention (e.g., CON193, CON166, CON103, CON203, CON198, CON197, CON202, CON222, CON215, or CON217). The chromosomal localization permits use of the GPCR polynucleotide sequences (including fragments, thereof) as chromosomal markers to assist with genome mapping and to provide markers for disease states. Chromosomal localization also permits correlation of the GPCR's of the invention with disease states in which aberrant activity of the GPCR is implicated, especially disease states that have previously linked (or will be linked) with mutations, polymorphisms, chromosomal rearrangements, and other chromosomal changes near the locus of the GPCR gene.

A. CON197

Chomosomal localization of the gene encoding CON197 (SEQ ID NO: 11) was determined using the Standford G3 Radiation Hybrid Panel (Research Genetics, Inc. Huntsville, Ala.). This panel contains 83 radiation hybrid clones of the entire human genome as created by the Stanford Human Gemone Center (Stanford, Calif.). PCR was carried out with each clone within the Hybrid Panel and the results were submitted to the Standford Human Genomic Center via e-mail for analysis (http://www.shgc.standford.edu/RH/rhserverformnew.html).

PCR reactions were carried out with the Expand Hi-Fi PCR System™ according the manufacturer's instructions (Roche Molecular Biochemicals, Indianapolis, Ind.). Primers, synthesized by Genosys Corp. (The Woodlands, Tex.), were designed to generate a 10 base pair fragment of CON197-encoding DNA in the presence of the appropriate genomic DNA. The forward primer, denoted as LW1332 (TCCTACTGTCATGAACCC; SEQ ID NO: 74), corresponded to nucleotides 396 through 413 of SEQ ID NO: 11. The reverse primer, denoted as LW1333 (CAGAAGAAGTTGTCCAGC; SEQ ID NO: 75), corresponded to the complement of nucleotides 519 through 536 of SEQ ID NO: 11. Each reaction contained 25 ng of DNA from a hybrid clone, 60 ng of Primer LW1332, and 60 ng of Primer LW1333 resulting in a final volume of 15 μl. The PCR reactions were carried our in a GeneAmp 9700 PCR thermocycler (Perkin Elmer Applied Biosystems) under the following conditions: 94° C. for 3 minutes followed by 35 cycles of 94° C. for 30 seconds, 52° C. for 1 minute, and 72° C. for 2 minutes. The PCR reactions were then analyzed on a 2.0% agarose gel and stained with ethidium bromide. The lanes were scored for the presence of the 140 base pair PCR product.

The G3 Hybrid Panel analysis revealed that the CON197 gene (SEQ ID NO: 11) was localized to chromosome 14, most nearly linked to Standford marker SHGC-10764 with a LOD score of 9.10. The SHGC- 10764 marker lies at position 1q11.1.

B. CON202

Chomosomal localization of the gene encoding CON202 (SEQ ID NO: 13) was determined using the Standford G3 Radiation Hybrid Panel (Research Genetics. Inc. Huntsville, Ala.). This panel contains 83 radiation hybrid clones of the entire human genome as created by the Stanford Human Gemone Center (Stanford, Calif.). PCR was carried out with each clone within the Hybrid Panel and the results were submitted to the Standford Human Genomic Center via e-mail for analysis (http://www.shgc.standford.edu/RH/rhserverformnew.html).

PCR reactions were carried out with the Expand Hi-Fi PCR System™ according the manufacturer's instructions (Roche Molecular Biochemicals, Indianapolis, Ind.). Primers, synthesized by Genosys Corp. (The Woodlands, Tex.), were designed to generate a 250 base pair fragment of CON202-encoding DNA in the presence of the appropriate genomic DNA. The forward primer, denoted as LW1480 (GGTTCTACCTGGACTTATGG; SEQ ID NO: 70), corresponded to nucleotides 515 through 534 of SEQ ID NO: 13. The reverse primer, denoted as LW1481 (TAATGAATGAGTAAGTGCCC; SEQ ID NO: 71), corresponded to the complement of nucleotides 745 through 764 of SEQ ID NO: 13. Each reaction contained 25 ng of DNA from a hybrid clone, 60 ng of Primer LW1480, and 60 ng of Primer LW1481 resulting in a final volume of 15 μl. The PCR reactions were carried our in a GeneAmp 9700 PCR thermocycler (Perkin Elmer Applied Biosystems) under the following conditions: 94° C. for 3 minutes followed by 35 cycles of 94° C. for 30 seconds, 52° C. for 1 minute, and 72° C. for 2 minutes. The PCR reactions were then analyzed on a 2.0% agarose gel and stained with ethidium bromide. The lanes were scored for the presence of the 250 base pair PCR product.

The G3 Hybrid Panal analysis revealed that the CON202 gene (SEQ ID NO: 13) was localized to chromosome 7, most nearly linked to Standford marker SHGC-12021 with a LOD score of 10.36. The SHGC-12021 marker lies at position 7q21. There is evidence that schizophrenia is linked to chromosome 7q22, and therefor any genes localized to this region are candidates for disease involvement or susceptibility. [See Ekelund et al., Human Mol. Genetics 9(7): 1049-1057 (2000); Faraone et al., Am. J. Med. Genet. 81: 290-295 (September, 1998); and Blouin et al., Nat. Genet., 20: 70-73(1998)]. The SHGC-12021 marker is proximal to 7q22 (˜1 cM) and therefore may be associated with schizophrenia susceptibility.

In particular, G protein-coupled receptors, such as CON202 polypeptide, have the biochemical and functional potential to play a role in the disease process of schizophenia. CON202 is an attractive target for screening for ligands (natural and synthetic) that are useful in modulating cellular processes involved in schizophrenia. In addition, the chromosomal localization data (especially coupled with CON202 expression patterns in the brain) identifies CON202 as a candidate for screening healthy and affected (schizophrenia) individuals for CON202 allelic variants, mutations, duplications, rearrangements, and other chromosomal variations that correlate with the disesase state. Variations that correlate with disease state are useful for diagnosis of disease or disease susceptibility. CON202 constructs containing the variations are useful for designing targeted therapeutics for treatment of the disease (e.g., by using the assays for modulators described in preceding examples.

C. High Throughput Analysis

The EMBL High Throughput Genome database (provided by the European Bioinformics Institute) was searched with GPCR nucleotide sequences to determine chromosomal localization for CON193, CON166, CON103, CON203, CON198, and CON215 genes. The results are summarized in the table below:

| | | Chomosome | Based on Genbank |
|---|---|---|---|
| GPCR | SEQ ID NO: | Localization | Accession No. |
| CON193 | 1 | 11 | AC026090 |
| CON166 | 3 | X | AC021992 |
| CON103 | 5 | 2 | AC013396 |
| CON203 | 7 | 3 | AC024886 |
| CON198 | 9 | 11 | AC025249 |
| CON215 | 17 | 3 | AC024886 |

While the present invention has been described in terms of specific embodiments, it is understood that variations and modifications will occur to those in the art, all of which are intended as aspects of the present invention. Accordingly, only such limitations as appear in the claims should be placed on the invention.

Summary of Sequences:

| SEQ ID | |
|---|---|
| NO. | Description |
| 1 | CON 193 DNA |
| 2 | CON 193 protein |
| 3 | CON 166 DNA |
| 4 | CON 166 protein |
| 5 | CON 103 DNA |
| 6 | CON 103 protein |
| 7 | CON 203 DNA |
| 8 | CON 203 protein |
| 9 | CON 198 DNA |
| 10 | CON 198 protein |
| 11 | CON 197 DNA |
| 12 | CON 197 protein |
| 13 | CON 202 DNA |
| 14 | CON 202 protein |
| 15 | CON 222 DNA |
| 16 | CON 222 protein |
| 17 | CON 215 DNA |
| 18 | CON 215 protein |
| 19 | CON 217 DNA |
| 20 | CON 217 protein |
| 21 | PCR primer LW 1282 for CON 193 |
| 22 | PCR primer LW 1283 for CON 193 |
| 23 | PCR primer LW 1372 for CON 193 |
| 24 | PCR primer LW 1374 for CON 193 |
| 25 | MAPK Substrate Peptide |
| 26 | Primer LW 1248 for CON 193 to generate insitu hybridization |
| | probe |
| 27 | Primer LW 1249 for CON 193 to generate insitu hybridization |
| | probe |
| 28 | PCR primer LW 1278 for CON 166 |
| 29 | PCR primer LW 1279 for CON 166 |
| 30 | PCR primer LW 1405 for CON 166 |
| 31 | PCR primer LW 1406 for CON 166 |
| 32 | PCR primer LW 1280 for CON 103 |
| 33 | PCR primer LW 1281 for CON 103 |
| 34 | PCR primer LW 1385 for CON 103 |
| 35 | PCR primer LW 1386 for CON 103 |
| 36 | PCR primer LW 1329 for CON 203 |
| 37 | PCR primer LW 1377 for CON 203 |
| 38 | PCR primer LW 1387 for CON 203 |
| 39 | PCR primer LW 1388 for CON 203 |
| 40 | Primer LW 1314 for CON 203 to generate insitu hybridization |
| | probe |
| 41 | Primer LW 1315 for CON 203 to generate insitu hybridization |
| | probe |
| 42 | PCR primer LW 1326 for CON 198 |
| 43 | PCR primer LW 1327 for CON 198 |
| 44 | PCR primer LW 1415 for CON 198 |
| 45 | PCR primer LW 1416 for CON 198 |
| 46 | Primer LW 1308 for CON 198 to generate insitu hybridization |
| | probe |
| 47 | Primer LW 1309 for CON 198 to generate insitu hybridization |
| | probe |
| 48 | PCR primer LW 1324 for CON 197 |
| 49 | PCR primer LW 1325 for CON 197 |
| 50 | Primer LW 1306 for CON 197 to generate insitu hybridization |
| | probe |
| 51 | Primer LW 1307 for CON 197 to generate insitu hybridization |
| | probe |
| 52 | PCR primer GV 599 for CON 202 |
| 53 | PCR primer GV 600 for CON 202 |
| 54 | PCR primer LW 1482 for CON 202 |
| 55 | PCR primer LW 148 for CON 202 |
| 56 | Primer LW 1310 for CON 202 to generate insitu hybridization |
| | probe |
| 57 | Primer LW 1311 for CON 202 to generate insitu hybridization |
| | probe |
| 58 | PCR primer LW 1442 for CON 222 |
| 59 | PCR primer LW 1443 for CON 222 |
| 60 | PCR primer LW 1440 for CON 222 |
| 61 | PCR primer LW 1441 for CON 222 |
| 62 | Primer LW 1472 for CON 222 to generate insitu hybridization |
| | probe |
| 63 | Primer LW 1473 for CON 222 to generate insitu hybridization |
| | probe |
| 64 | Primer LW 1411 for CON 215 to generate insitu hybridization |
| | probe |
| 65 | Primer LW 1412 for CON 215 to generate insitu hybridization |
| | probe |
| 66 | PCR primer LW 1448 for CON 217 |
| 67 | PCR primer LW 1449 for CON 217 |
| 68 | Primer LW 217A for CON 217 to generate insitu hybridization |
| | probe |
| 69 | Primer LW 218B for CON 217 to generate insitu hybridization |
| | probe |
| 70 | Primer LW 1480 for CON 202 chromosomal localization |
| 71 | Primer LW 1481 for CON 202 chromosomal localization |
| 72 | Primer CON103a for CON 103 to generate insitu hybridization |
| | probe |
| 73 | Primer CON103b for CON 103 to generate insitu hybridization |
| | probe |
| 74 | Primer LW 1332 for CON 197 chromosomal localization |
| 75 | Primer LW 1333 for CON 197 chromosomal localization |

What is claimed is:

1. A purified and isolated seven transmembrane receptor polypeptide comprising an amino acid sequence at least 90% identical to an amino acid sequence set forth in any one of SEQ ID NOS: 2, 4, 6, 8, 10, 12, 14, 16, 18 or 20, or a fragment thereof comprising an epitope specific to said seven transmembrane receptor polypeptide.

2. A purified and isolated seven transmembrane receptor polypeptide according to claim 1 comprising an amino acid sequence at least 90% identical to the amino acid sequence set forth in SEQ ID NO: 2, or a fragment thereof comprising an epitope specific to said seven transmembrane receptor polypeptide.

3. A purified and isolated seven transmembrane receptor polypeptide according to claim 1 comprising an amino acid sequence at least 90% identical to the amino acid sequence set forth in SEQ ID NO: 4, or a fragment thereof comprising an epitope specific to said seven transmembrane receptor polypeptide.

4. A purified and isolated seven transmembrane receptor polypeptide according to claim 1 comprising an amino acid sequence at least 90% identical to the amino acid sequence set forth in SEQ ID NO: 6, or a fragment thereof comprising an epitope specific to said seven transmembrane receptor polypeptide.

5. A purified and isolated seven transmembrane receptor polypeptide according to claim 1 comprising an amino acid sequence at least 90% identical to the amino acid sequence set forth in SEQ ID NO: 8, or a fragment thereof comprising an epitope specific to said seven transmembrane receptor polypeptide.

6. A purified and isolated seven transmembrane receptor polypeptide according to claim 1 comprising an amino acid sequence at least 90% identical to the amino acid sequence set forth in SEQ ID NO: 10, or a fragment thereof comprising an epitope specific to said seven transmembrane receptor polypeptide.

7. A purified and isolated seven transmembrane receptor polypeptide according to claim 1 comprising an amino acid sequence at least 90% identical to the amino acid sequence set forth in SEQ ID NO: 12, or a fragment thereof comprising an epitope specific to said seven transmembrane receptor polypeptide.

8. A purified and isolated seven transmembrane receptor polypeptide according to claim 1 comprising an amino acid sequence at least 90% identical to the amino acid sequence set forth in SEQ ID NO: 14, or a fragment thereof comprising an epitope specific to said seven transmembrane receptor polypeptide.

9. A purified and isolated seven transmembrane receptor polypeptide according to claim 1 comprising an amino acid sequence at least 90% identical to the amino acid sequence set forth in SEQ ID NO: 16, or a fragment thereof comprising an epitope specific to said seven transmembrane receptor polypeptide.

10. A purified and isolated seven transmembrane receptor polypeptide according to claim 1 comprising an amino acid sequence at least 90% identical to the amino acid sequence set forth in SEQ ID NO: 18, or a fragment thereof comprising an epitope specific to said seven transmembrane receptor polypeptide.

11. A purified and isolated seven transmembrane receptor polypeptide according to claim 1 comprising an amino acid sequence at least 90% identical to the amino acid sequence set forth in SEQ ID NO: 20, or a fragment thereof comprising an epitope specific to said seven transmembrane receptor polypeptide.

12. A purified and isolated seven transmembrane receptor polypeptide according to any one of claims 1-11.

13. A purified and isolated polypeptide according to any one of claims 1-11 comprising at least one extracellular domain of the seven transmembrane receptor polypeptide.

14. A purified and isolated polypeptide according to any one of claims 1-11 comprising the N-terminal extracellular domain of the seven transmembrane receptor polypeptide.

15. A purified and isolated polypeptide according to any one of claims 1-11 comprising a seven transmembrane receptor fragment selected from the group consisting of an N-terminal extracellular domain transmembrane domains, extracellular loops connecting transmembrane domains, intracellular loops connecting transmembrane domains, a C-terminal cytoplasmic domain, and fusions thereof.

16. A polypeptide according to any one of claims 1-15, wherein the polypeptide further includes a heterologous tag amino acid sequence.

17. A purified and isolated polynucleotide comprising a nucleotide sequence that encodes the polypeptide of claim 16.

18. A purified and isolated polynucleotide comprising a nucleotide sequence that encodes a polypeptide according to any one of claim 2, 3, 4, 8 or 9.

19. A purified and isolated polynucleotide comprising a heterologous expression control sequence operatively linked to a nucleotide sequence that encodes a polypeptide according to any one of claims 1-16.

20. The polynucleotide according to claim 19 wherein the expression control sequence is a promoter sequence that promotes expression of said polynucleotide in an eukaryotic cell.

21. The polynucleotide according to claim 19, wherein the promoter is a heterologous promoter that promotes expression of the polynucleotide in a human cell.

22. A purified and isolated polynucleotide comprising a nucleotide sequence that encodes a mammalian seven transmembrane receptor, wherein said polynucleotide hybridizes to any one of the nucleotide sequences set forth in SEQ ID NOS: 1, 3, 5, 7, 9, 11, 13, 15, 17, or 19 or the non-coding strand complementary thereto, under the following hybridization conditions:
(a) hybridization for 16 hours at 42° C. in a hybridization solution comprising 50% formamide, 1% SDS, 1 M NaCl, 10% dextran sulfate and
(b) washing 2 times for 30 minutes at 60° C. in a wash solution comprising 0.1×SSC and 1% SDS, with the proviso that the nucleotide sequence of the polynucleotide differs from the coding sequence set forth in any one of SEQ ID NOS: 1, 3, 5, 7, 9, 11, 13, 15, 17, or 19 and from its complementary strand by at least one nucleotide.

23. A polynucleotide according to claim 22 that encodes a human seven transmembrane receptor.

24. A vector comprising a polynucleotide according to any one of claims 17-23.

25. A vector according to claim 24 that is an expression vector for expressing the polynucleotide in a mammalian cell.

26. A host cell stably transformed or transfected with a polynucleotide according to any one of claims 17-23 in a manner allowing the expression in said host cell of the polypeptide or fragment thereof encoded by the polynucleotide.

27. A host cell stably transformed or transfected with a vector according to claim 24 or 25 in a manner allowing the expression in said host cell of the polypeptide or fragment thereof encoded by the polynucleotide.

28. A method for producing a seven transmembrane receptor polypeptide comprising the steps of growing a host cell according to claim 26 or 27 in a nutrient medium under conditions in which the host cell expresses a seven transmembrane receptor encoded by the polynucleotide.

29. A method according to claim 28, further comprising a step of isolating said polypeptide from said cell or said medium.

30. A method according to claim 29, further comprising a step of isolating cell membranes from the host cell, wherein the cell membrane comprises the seven transmembrane receptor.

31. An antibody specific for a polypeptide according to any one of claims 1-15.

32. The antibody of claim 31 which is a monoclonal antibody.

33. A hybridoma that produces an antibody according to claim 32.

34. An antibody according to claim 31 that is a humanized antibody.

35. An antibody according to claim 31 that specifically binds an extracellular epitope of a seven transmembrane receptor having an amino acid sequence selected from the group consisting of SEQ ID NOS: 2, 4, 6, 8, 10, 12, 14, 16, 18 or 20.

36. An antibody according to claim 35 that specifically binds to the amino-terminal extracellular domain of the seven transmembrane receptors.

37. A cell-free composition comprising polyclonal antibodies, wherein at least one of said antibodies is an antibody according to claim 31.

38. An anti-idiotypic antibody specific for an antibody according to claim 31.

39. A polypeptide comprising a fragment of an antibody according to claim 31, wherein said fragment and said polypeptide specifically bind to a seven transmembrane receptor having an amino acid sequence selected from the group consisting of SEQ ID NOS: 2, 4, 6, 8, 10, 12, 14, 16, 18 or 20.

40. A polypeptide according to claim 39 that is selected from the group consisting of single chain antibodies and CDR-grafted antibodies.

41. A composition comprising a polypeptide according to any one of claims 1-16 in a pharmaceutically acceptable carrier.

42. A composition comprising an antibody according to any one of claims 31, 32, 34, 35, or 36, or a polypeptide according to claim 39 or 40, in a pharmaceutically acceptable carrier.

43. A method for modulating ligand binding of a seven transmembrane receptor polypeptide according to any one of claims 1-15, comprising the step of contacting said seven transmembrane receptor polypeptide with an antibody specific for said seven transmembrane receptor, under conditions wherein the antibody binds the receptor.

44. A method for modulating ligand binding of a seven transmembrane receptor polypeptide comprising the step of contacting said seven transmembrane receptor polypeptide with a polypeptide according to claim 39 or 40.

45. An assay to identify compounds that bind a seven transmembrane receptor polypeptide, said assay comprising the steps of:
(a) contacting a composition comprising a seven transmembrane receptor polypeptide according to any of claims 1-15 with a compound suspected of binding the seven transmembrane receptor polypeptide; and
(b) measuring binding between the compound and the seven transmembrane receptor polypeptide.

46. A method for identifying a modulator of binding between a seven transmembrane receptor polypeptide and a binding partner of the seven transmembrane receptor polypeptide, comprising the steps of:
(a) contacting the binding partner and a composition comprising the seven transmembrane receptor polypeptide in the presence and in the absence of a putative modulator compound, where the seven transmembrane receptor polypeptide is a polypeptide according to any one of claims 1-15;
(b) measuring binding between the binding partner and said seven transmembrane receptor polypeptide; and
(c) identifying a putative modulator compound in view of decreased or increased binding between the binding partner and seven transmembrane receptor polypeptide in the presence of the putative modulator, as compared to binding in the absence of the putative modulator.

47. An assay according to claim 45 or 46 wherein the composition comprises a cell expressing the seven transmembrane receptor polypeptide on its surface.

48. An assay according to claim 47 wherein the measuring step comprises measuring intracellular signaling of the seven transmembrane receptor polypeptide induced by the compound.

49. A method for treating a neurological disorder comprising the step of administering to a mammal in need of such treatment a pharmaceutical composition comprising a compound in an amount effective to modulate biological activity of a seven transmembrane receptor in neurons of said mammal, wherein the compound is selected from the group consisting of:
(a) an antibody according to any one of claim 31, 32, 34, 35, or 36;
(b) an anti-idiotypic antibody according to claim 38;
(c) a polypeptide according to claim 39 or 40;
(d) a compound identified according to the method of claim 45; and
(e) a modulator identified according to claim 46.

50. The method of claim 49 wherein the neurological disorder is schizophrenia.

51. A method according to claim 50, wherein the seven transmembrane receptor comprises a polypeptide according to claim 8.

52. A method of treating schizophrenia comprising the step of administering to a human diagnosed with schizophrenia an amount of a modulator of CON202 receptor activity sufficient to modulate CON202 receptor activity or CON202 ligand binding in said human.

53. A method of diagnosing schizophrenia or a susceptibility to schizophrenia comprising the steps of:
(a) measuring the presence or amount of expression or activity of a polypeptide according to claim 8 in a cell of a human patient: and
(b) comparing the measurement of step (a) to a measurement of expression or activity of the polypeptide in a cell from a normal subject or the patient at an earlier time, wherein the diagnosis of schizophrenia or susceptibility to schizophrenia is based on the presence or amount of CON202 polypeptide expression or activity.

54. A method of screening a human subject to diagnose a disorder affecting the brain or genetic predisposition therefor, comprising the steps of:
(a) assaying nucleic acid of a human subject to determine a presence or an absence of a mutation altering the amino acid sequence, expression, or biological activity of at least one seven transmembrane receptor that is expressed in the brain, wherein the seven transmembrane receptor comprises an amino acid sequence selected from the group consisting of SEQ ID NOs: 2, 4, 6, 8, 10, 12, 14, 16, 18, and 20, or an allelic variant thereof, and wherein the nucleic acid corresponds to the gene encoding the seven transmembrane receptor; and
(b) diagnosing the disorder or predisposition from the presence or absence of said mutation, wherein the presence of a mutation altering the amino acid sequence, expression, or biological activity of allele in the nucleic acid correlates with an increased risk of developing the disorder.

55. A method according to claim 54, wherein the seven transmembrane receptor is CON202 comprising an amino acid sequence set forth in SEQ ID NO: 14, or an allelic variant thereof.

56. A method according to claim 55, wherein the disease is schizophrenia.

57. A method according to claim 56, wherein the assaying step comprises at least one procedure selected from the group consisting of:
(a) determining a nucleotide sequence of at least one codon of at least one CON202 allele of the human subject;
(b) performing a hybridization assay to determine whether nucleic acid from the human subject has a nucleotide sequence identical to or different from one or more reference sequences;
(c) performing a polynucleotide migration assay to determine whether nucleic acid from the human subject has a nucleotide sequence identical to or different from one or more reference sequences; and
(d) performing a restriction endonuclease digestion to determine whether nucleic acid from the human subject has a nucleotide sequence identical to or different from one or more reference sequences.

58. A method according to claim 56 wherein the assaying step comprises: performing a polymerase chain reaction (PCR) to amplify nucleic acid comprising CON202 coding sequence, and determining nucleotide sequence of the amplified nucleic acid.

59. A method of screening for a CON202 hereditary schizophrenia genotype in a human patient, comprising the steps of:
(a) providing a biological sample comprising nucleic acid from said patient, said nucleic acid including sequences corresponding to said patient's CON202 alleles;
(b) analyzing said nucleic acid for the presence of a mutation or mutations;
(c) determining a CON202 genotype from said analyzing step; and
(d) correlating the presence of a mutation in a CON202 allele with a hereditary schizophrenia genotype.

60. The method according to claim 59 wherein said biological sample is a cell sample.

61. The method according to claim 59 wherein said analyzing comprises sequencing a portion of said nucleic acid, said portion comprising at least one codon of said CON202 alleles.

62. The method according to claim 59 wherein said nucleic acid is DNA.

63. The method according to claim 59 wherein said nucleic acid is RNA.

64. A kit for screening a human subject to diagnose schizophrenia or a genetic predisposition therefor, comprising, in association:
(a) an oligonucleotide useful as a probe for identifying polymorphisms in a human CON202 seven transmembrane receptor gene, the oligonucleotide comprising 6-50 nucleotides that have a sequence that is identical or exactly complementary to a portion of a wild type human CON202 gene sequence or CON202 coding sequence, except for one sequence difference selected from the group consisting of a nucleotide addition, a nucleotide deletion, or nucleotide substitution; and
(b) a media packaged with the oligonucleotide containing information identifying polymorphisms identifyable with the probe that correlate with schizophrenia or a genetic predisposition therefor.

65. A method of identifying a seven transmembrane allelic variant that correlates with a mental disorder, comprising, steps of:
(a) providing a biological sample comprising nucleic acid from a human patient diagnosed with a mental disorder, or from the patient's genetic progenitors or progeny;
(b) analyzing said nucleic acid for the presence of a mutation or mutations in at least one seven transmembrane receptor that is expressed in the brain, wherein the at least one seven transmembrane receptor comprises an amino acid sequence selected from the group consisting of SEQ ID NOs: 2, 4, 6, 8, 10, 12, 14, 16, 18, and 20, or an allelic variant thereof, and wherein the nucleic acid includes sequence corresponding to the gene or genes encoding the at least one seven transmembrane receptor;
(c) determining a genotype for the patient for the at least one seven transmembrane receptor from said analyzing step; and
(d) identifying an allelic variant that correlates with the mental disorder from the determining step.

66. A method according to claim 65, wherein the disorder is schizophrenia, and wherein the at least one seven transmembrane receptor comprises CON202 having an amino acid sequence set forth in SEQ ID NO: 14, or an allelic variant thereof.

67. A purified and isolated polynucleotide comprising a nucleotide sequence encoding a CON202 receptor allelic variant identified according to claim 66.

68. A host cell transformed or transfected with a polynucleotide according to claim 67 or with a vector comprising the polyncleotide.

69. A purified polynucleotide comprising a nucleotide sequence encoding a CON202 seven transmembrane receptor protein of a human that is affected with schizophrenia;
wherein said polynucleotide hybridizes to the complement of SEQ ID NO: 13 under the following hybridization conditions:
(a) hybridization for 16 hours at 42° C. in a hybridization solution comprising 50% formamide, 1% SDS, 1 M NaCl, 10% dextran sulfate and
(b) washing 2 times for 30 minutes at 60° C. in a wash solution comprising 0.1×SSC and 1% SDS; and
wherein the polynucleotide encodes a CON202 amino acid sequence that differs from SEQ ID NO: 14 at at least one residue.

70. A vector comprising a polynucleotide according to claim 69.

71. A host cell that has been transformed or transfected with a polynucleotide according to claim 70 and that expresses the CON202 protein encoded by the polynucleotide.

72. A host cell according to claim 71 that has been co-transfected with a polynucleotide encoding the CON202 amino acid sequence set forth in SEQ ID NO: 14 and that expresses the con202 protein having the amino acid sequence set forth in SEQ ID NO: 14.

73. A method for identifying a modulator of CON202 biological activity, comprising the steps of:
a) contacting a cell according to claim 71 in the presence and in the absence of a putative modulator compound;
b) measuring CON202 biological activity in the cell; and
c) identifying a putative modulator compound in view of decreased or increased CON202 biological activity in the presence versus absence of the putative modulator.

74. An assay to identify compounds useful for the treatment of schizophrenia, said assay comprising steps of:
(a) contacting a composition comprising a seven transmembrane receptor polypeptide according to claim 8 with a compound suspected of binding the seven transmembrane receptor polypeptide;
(b) measuring binding between the compound and the seven transmembrane receptor polypeptide; and
(c) identifying molecules that bind the seven transmembrane receptor as candidate compounds useful for the treatment of schizophrenia.

75. A method for identifying compound useful for a modulator of binding between a seven transmembrane receptor polypeptide and a binding partner of the seven transmembrane receptor polypeptide, which modulator is useful for treatment of schizophrenia, comprising the steps of:
(a) contacting the binding partner and a composition comprising the seven transmembrane receptor polypeptide in the presence and in the absence of a putative modulator compound, where the seven transmembrane receptor polypeptide is a polypeptide according to claim 8;
(b) measuring binding between the binding partner and the seven transmembrane receptor polypeptide;
(c) identifying a modulator compound useful for the treatment of schizophrenia in view of decreased or increased binding between the binding partner and seven transmembrane receptor polypeptide in the presence of the putative modulator, as compared to binding in the absence of the putative modulator.

76. An assay according to claim 74 or 75 wherein the composition comprises a cell expressing the seven transmembrane receptor polypeptide on its surface.

77. An assay according to claim 76 wherein the composition comprises a cell transformed or transfected with a polynucleotide encoding the seven transmembrane polypeptide and expressing the seven transmembrane receptor polypeptide on its surface.
